(19) United States
(12) Patent Application Publication
(10) Pub. No.: US 2026/0345999 A1
(43) Pub. Date: Nov. 5, 2026

(54) DEVICES AND SYSTEMS FOR A SINGLE OR MULTI CONTACT HELICAL WIRE STRUCTURES AND THEIR CONNECTORS

(71) Applicant: NEURONOFF, INC., Cleveland, OH (US)

(72) Inventors: Stephan NIEUWOUDT, Cleveland, OH (US); Manfred FRANKE, Cleveland, OH (US)

(21) Appl. No.: 19/734,377

(22) Filed: Jul. 8, 2026

Related U.S. Application Data
(63) Continuation-in-part of application No. 19/685,160, filed on May 22, 2026.
(63) Continuation-in-part of application No. 19/468,229, filed on Feb. 3, 2026.
(63) Continuation-in-part of application No. 19/456,700, filed on Jan. 22, 2026.
(63) Continuation-in-part of application No. 19/191,595, filed on Apr. 28, 2025.
(63) Continuation-in-part of application No. 18/561,425, filed on Nov. 16, 2023.
Application No. 18/561,425 is a continuation-in-part of application No. 18/285,349, filed on Oct. 2, 2023, now Pat. No. 12,642,575.
Application No. 18/285,349 is a continuation-in-part of application No. 18/278,160, filed on Aug. 21, 2023.
Application No. 18/278,160 is a continuation-in-part of application No. 18/026,705, filed on Mar. 16, 2023.
(60) Provisional application No. 63/167,836, filed on Mar. 30, 2021.
(60) Provisional application No. 63/171,780, filed on Apr. 7, 2021.
(60) Provisional application No. 63/184,656, filed on May 5, 2021.
(60) Provisional application No. 63/226,458, filed on Jul. 28, 2021.
(60) Provisional application No. 63/236,678, filed on Aug. 24, 2021.
(60) Provisional application No. 63/306,896, filed on Feb. 4, 2022.
(60) Provisional application No. 63/844,854, filed on Jul. 16, 2025.

(51) Int. Cl. A61N 1/05; A61B 17/00; A61B 17/3211

(52) U.S. Cl. CPC A61N 1/0504; A61B 17/3211; A61B 2017/00349

(57) ABSTRACT

A helical wire structure electrode with a loopback configuration includes a single wire rope structure having two ends and a middle region. Two proximal electrodes are formed by uncoated regions at each end of the wire rope structure. One distal electrode is formed by an uncoated offset region located near the middle of the wire rope structure. The wire rope structure is folded back upon itself at the uncoated offset region to produce two parallel ropes with the two proximal electrodes offset from each other so as to not directly overlap. The folded wire rope structure is wound on a core to produce a helical wire loopback structure electrode.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### CROSS-REFERENCE TO RELATED APPLICATIONS

This application claims the benefit of U.S. Provisional Patent Application No. 63/844,854 filed on Jul. 16, 2025, which is incorporated by reference herein in its entirety.

This application is a Continuation-In-Part Utility patent application claiming priority to U.S. patent application Ser. No. 19/685,160, filed on May 5, 2026.

This application is a Continuation-In-Part Utility patent application claiming priority to U.S. patent application Ser. No. 19/468,229, filed on Feb. 3, 2026.

This application is a Continuation-In-Part Utility patent application claiming priority to U.S. patent application Ser. No. 19/191,595, filed on Apr. 28, 2025.

This application is a Continuation-In-Part Utility patent application claiming priority to U.S. patent application Ser. No. 18/561,425, filed on Nov. 16, 2023.

This application is a Continuation-In-Part Utility patent application claiming priority to U.S. patent application Ser. No. 19/456,700 filed on Jan. 22, 2026.

This application is a Continuation-In-Part Utility patent application claiming priority to U.S. patent application Ser. No. 18/285,349, U.S. Pat. No. 12,642,575, filed on Oct. 2, 2023 and issued on Jun. 2, 2026.

U.S. patent application Ser. No. 18/285,349, is a Continuation-In-Part Utility patent application claiming priority to U.S. patent application Ser. No. 18/278,160, filed on Aug. 21, 2023.

U.S. patent application Ser. No. 18/285,349, is a Continuation-In-Part Utility patent application claiming priority to U.S. patent application Ser. No. 18/026,705, filed on Mar. 16, 2023.

U.S. patent application Ser. No. 18/285,349, is a is a National Stage Entry of PCT/US2022/029918, filed on May 18, 2022.

U.S. patent application Ser. No. 18/285,349, is a is a National Stage Entry of PCT/US2022/020652, filed on Mar. 16, 2022.

PCT/US2022/020652 claims the benefit of U.S. Provisional Patent Application No. 63/306,896, filed on Feb. 4, 2022.

PCT/US2022/029918 claims the benefit of U.S. Provisional Patent Application No. 63/236,678, filed on Aug. 24, 2021.

PCT/US2022/029918 claims the benefit of U.S. Provisional Patent Application No. 63/226,458, filed on Jul. 28, 2021.

U.S. patent application Ser. No. 18/026,705, is a is a National Stage Entry of PCTUS2021033007, filed on May 18, 2021.

PCT/US2022/020652 claims the benefit of U.S. Provisional Patent Application No. 63/184,656, filed on May 5, 2021.

PCT/US2022/020652 claims the benefit of U.S. Provisional Patent Application No. 63/171,780, filed on Apr. 7, 2021.

PCT/US2022/020652 claims the benefit of U.S. Provisional Patent Application No. 63/167,836, filed on Mar. 30, 2021.

All of which are incorporated by reference herein in their entirety.

### BACKGROUND OF THE INVENTION

### 1. Field of the Invention

The present disclosure relates to helical wire structure electrodes and connector systems for electrical stimulation applications, and more particularly to multi-contact helical wire structure electrode configurations, loopback electrode designs, connector systems for interfacing helical wire structure electrodes with pulse generators, and devices for the percutaneous removal of injectable electrodes.

Implantable electrodes for electrical stimulation have been used in various medical applications, including pain management, treatment of neurological conditions, and other therapeutic interventions. These electrodes are typically designed to deliver electrical signals to target tissues such as nerves, muscles, or other anatomical structures within the body.

Existing electrode systems often involve complex surgical procedures for implantation, which may increase patient discomfort, extend recovery times, and introduce potential complications. Traditional electrode designs may also present challenges related to anchoring and stabilization within tissue, as electrodes that migrate from their intended placement location may result in suboptimal therapeutic outcomes.

Connecting implanted electrodes to pulse generators or other stimulation devices presents additional challenges. Conventional connection systems may involve multiple components, specialized tools, or intricate procedures that add complexity to the implantation process. In some cases, previously implanted electrodes may need to be connected to new or different pulse generators, which can present difficulties when the original electrode design does not readily accommodate such connections.

Multi-contact electrode configurations, which allow for stimulation at multiple tissue sites or provide flexibility in stimulation patterns, may involve manufacturing complexities and connection challenges that differ from single-contact designs. The ability to interface multiple electrode contacts with pulse generators while maintaining reliable electrical connections and appropriate insulation between contacts remains an area of ongoing development.

When implanted electrodes need to be removed, whether due to infection, device malfunction, or other clinical considerations, the removal procedure may present its own set of challenges. Electrodes that have been implanted for extended periods may become integrated with surrounding tissue, and removal procedures may benefit from specialized tools designed to facilitate electrode retrieval while minimizing tissue trauma.

Accordingly, there remains a general interest in electrode systems and related devices that may address one or more of these considerations in the field of implantable electrical stimulation.

These and other objects, features, and advantages of the present invention will become more readily apparent from the attached drawings and the detailed description of the preferred embodiments, which follow.

### SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a helical wire structure electrode with a loopback configuration is provided. The helical wire structure electrode includes two exposed electrodes at a proximal end configured for placement on an electrical stimulation target and one exposed electrode at a distal location configured to serve as a collector. The helical wire structure electrode is fabricated from a single wire rope structure with exposed ends that form the proximal electrodes and an exposed region offset from a center of the wire rope structure that forms the distal electrode. The wire rope structure is folded back at the distal electrode to produce two parallel ropes with the proximal electrodes offset from each other so as to not directly overlap. The resulting U-shaped parallel rope wire structure is wound on a core to produce the helical wire structure electrode with the loopback design.

According to other aspects of the present disclosure, the helical wire loopback structure electrode may be reversed so that the loopback central electrode is placed on the stimulation target proximally, producing a helical wire loopback structure electrode with one proximal electrode and two distal electrodes. Two or more helical wire loopback structures may be combined by offsetting the loopback rope wire structures at a predetermined distance, placing them in parallel, and winding the parallel structure around a mandrel.

According to another aspect of the present disclosure, a method for adding a connector to a previously implanted helical wire structure electrode without complete removal from a body is provided. The method includes partially extracting a distal component of the implanted helical wire structure electrode under skin to extend the structure back to a non-helical rope wire structure configuration. The partially elongated electrode end is cut to length, and a cylindrical contact is applied by threading the wire rope structure into the cylinder. A crimping force or a predetermined swaging diameter reduction is applied to the cylindrical contact to mechanically and electrically secure the cylinder to the electrode. The helical wire structure electrode is thereby converted without complete removal to an electrode that can be placed into a socket connector of an implantable pulse generator.

According to another aspect of the present disclosure, an integrated multi-contact pin and socket connector system for a multi-contact helical wire structure electrode is provided. The system includes multiple cylindrical connectors on a distal end of the multi-contact helical wire structure electrode. The cylindrical conductive contacts are separated by an insulative material from each other. Individual conductors of the multi-contact helical wire structure electrode are connected to each individual cylindrical contact through welding, brazing, or using a conductive adhesive. The insulative material may partially extend into a remainder of the helical wire structure. The multi-connector system may be used for electrical stimulation or other applications such as radiofrequency ablation.

According to other aspects of the present disclosure, a multi-contact helical wire structure electrode may include ring contacts compatible with implantable pulse generator headers. A standard and injectable electrode hybrid configuration may include a leading helical wire structure that allows for formation of an anchoring bundle upon device placement, standard lead ring contact electrodes, a standard lead section, and a central helical wire structure where connection wires from the standard ring contacts are routed through an open helical coil structure. The open helical coil structure may include multitude (2, 3, 4, 5, 6, 7, 8, 9 or 10 or more) parallel wire rope structures that are independently arranged to allow for independent mechanical separation from the primary helical coil structure, or a multitude of parallel wire rope structures that are combined into a single sheath to limit independent movement and separation from the helical coil structure of each wire rope structure.

According to another aspect of the present disclosure, manufacturing methods for multi-contact injectable electrodes are provided. An overwound dual contact injectable electrode may use two mandrels, one for a leading electrode contact and another for a remainder of the helical wire structure beyond a second contact tissue interface. In a region between the two mandrels, a coated wire structure of the leading electrode spans a distance of the second electrode contact and serves as the mandrel in that region. For contacts beyond two in a bipolar configuration, a distance of a gap between the two mandrels may be extended to accommodate additional leads.

According to another aspect of the present disclosure, a split dual contact injectable electrode is provided. The split dual contact injectable electrode includes helical wire structure contacts and split wire rope structures extending from the electrode body. The split wire rope structures may include uncoated regions that provide exposed conductive surfaces for electrical connection. A delivery system for the split contact injectable electrode may include an external cannula for delivery, an internal delivery cannula having an inner diameter smaller than a diameter of the helical wire structure electrode but greater than two parallel rope wire structures, and split wire rope structures extending beyond the internal delivery cannula for interfacing during or after placement.

According to another aspect of the present disclosure, interfacing solutions for injectable electrodes are provided. A transcutaneous interface may include an injectable electrode interfaced with a dual contact skin adhesive conductive hydrogel patch onto which an external pulse generator connects. A percutaneous interface may include injectable electrodes that are either two monopolar electrodes or a single bipolar electrode connected by conductive hook-and-loop connections that secure the electrodes and provide electrical connection between the electrodes and an external pulse generator output via an adhesive patch adapter.

According to another aspect of the present disclosure, a hook blade device for percutaneous removal of an injectable electrode is provided. The hook blade includes a cutting edge curvature on an outer edge for performing primary skin and tissue cutting down to a target electrode, and a hook on a non-cutting edge of the blade configured to engage with and retrieve the electrode. The hook has a diameter that is at least 1.25 times a diameter of the target electrode and up to 10 times the diameter. An inner edge of the hook has a sharpness level that cuts tissue but does not cut the electrode, while an outer edge is sharper than the inner edge to facilitate tissue cutting. Using imaging, alignment of the hook blade and confirmation of electrode engagement within the hook may be procedurally confirmed.

The hook blade device may be electrically connected to an impedance analyzer that differentiates contact with tissue versus contact with an implanted electrode by changes in measured impedance with respect to a distal return (patch or needle) or circuit return included as a separate conductive feature on the face or edge of the blade in a region outside of the hook. The impedance analyzer may sweep between different frequencies (10 Hz, 100 Hz, 1 kHz, 10 kHz, 100 kHz, 1 MHz) for which the determined impedance and electrical impedance spectroscopy plot will differ between tissue only contact and contact with a conductive electrode. An audible and/or visual signal will then supplement the tactile feedback clinicians normally rely on when performing percutaneous surgical removal of electrodes.

In addition to or as an alternative to electrical impedance based detection, audible and ultrasound frequency based detection may be performed directly through the hook blade device. When the hook blade contacts the implanted electrode, the scraping of the blade against the metallic structure of the electrode produces high frequency oscillations that are either audibly perceived as scratching, or physically perceived as vibrations. Connecting a piezoelectric sensor (audible or ultrasound frequency ranges from 10 Hz to 15 MHz) directly to the blade provides the input data to a digital analyzer that determines whether contact has or has not been with an electrode. Audible and/or visual feedback is then provided to the clinical user.

The foregoing general description of the illustrative embodiments and the following detailed description thereof are merely exemplary aspects of the teachings of this disclosure and are not restrictive.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows a gold wire rope (pre-helical structure) (upper) and an electrode with the helical wire structure (lower) made from the wire rope.

FIG. 2-A is an image (15.6×) of one embodiment of the injectable electrode with the helical wire structure with the guidewire, and FIG. 2-B is the same structure (7.4×) after removal of the guidewire.

FIG. 3-A is a close up of a rounded end of the electrode in FIGS. 2-A and 2-B, and 3-B is a close up (100×) of a middle portion of the same.

FIG. 4-A is an image (14.3×) of one embodiment of the injectable electrode with the helical wire structure comprising 50 strands of 25 micron diameter gold wire and 5 wires of 75 micron diameter gold wire. FIG. 4-B is an embodiment with the helical wire structure comprising 10 strands of 75 micron diameter gold wire.

FIG. 5-A is a closer image (100×) of the image in FIGS. 4-A, and 5-B is a closer image (100×) of the image in FIG. 4-B.

FIG. 6-A is an image of an embodiment of the electrode comprising 100 strands of 25 micron diameter gold wire, each with a guidewire of 0.45 mm diameter, and 6-B is on a guidewire of 0.8 mm.

FIG. 7-A is an image (300×) of a latitudinal cross-section of an electrode comprising a helical wire structure comprising a wire rope comprising 100 strands of 25 micron diameter gold wire. This shows the hollow core. FIG. 7-B is the same electrode before the 0.25 mm guide wire was removed.

FIG. 8 is an image (150×) of a longitudinal cross section of the same electrode as in FIG. 7-A.

FIG. 9-A is an image (200×) of an electrode similar to HA and IA but manufactured on a larger guidewire, 0.45 mm, and FIG. 9-B on a guidewire of 0.8 mm.

FIG. 10 is an image (300×) of a latitudinal cross-section of an electrode comprising a helical wire structure comprising a wire rope comprising 50 strands of 25 micron diameter gold wire and 5 strands of 75 micron diameter gold wire, with a hollow core.

FIG. 11 is a schematic diagram of the helical wire structure electrode 1 as implanted with a first bunching anchor 8 at a tissue target such as a nerve, with a second bunching anchor 8 in the subcutaneous region, acting also as a connector pad, which is able to receive transcutaneous transmission of electrical current from a TENS unit 32 on the skin surface. All of the details of the electrode are not shown.

FIG. 11A is the same as FIG. 11, except that an additional bunching anchor has been deposited in the inter-muscle fascia tissue layer. All of the details of the electrode are not shown.

FIG. 11B is the same as FIG. 11, except that energy is being delivered by a probe to the bunching anchor serving as a connector pad. All of the details of the electrode are not shown.

FIG. 12-A is an image (20×) of an electrode, comprising 100 strands of 25 micron diameter gold wire with an outer diameter of 0.75 mm, injected and bunched as an anchor into medical gelatin (00 hardness), also having a substantially linear portion. FIG. 12-B is a close up image (80×) of part of the bunched anchoring portion.

FIG. 13-A is a fluoroscopic image of insertion of a needle to a feline dorsal root ganglion. In FIG. 13-B the electrode has begun to be pushed from the needle tip. FIG. 13-C shows the electrode beginning to bunch, and FIG. 13-D shows additional bunching.

FIG. 14-A, FIG. 14-B, FIG. 14-C and FIG. 14-D are fluoroscopy images showing a sequence of injection of the electrode comprising a helical wire structure and formation of a bunching anchor and substantially linear portion.

FIG. 15 is an image (200×) of a bunched anchoring portion of an electrode, comprising 100 strands of 25 micron diameter gold wire with an outer diameter of 0.75 mm, having been pulled and removed from a laboratory animal after implantation on a brachial plexus for 60 days.

FIG. 16-A, FIG. 16-B and FIG. 16-C show the difference in bunching anchors among helical wire structure electrodes comprising wire ropes of different compositions.

FIG. 17-A is an image of an electrode with a curved portion within the connective tissue cavity around a rodent sciatic nerve. FIG. 17-B is an image of the same implanted electrode taken from a different angle.

FIG. 18-A shows a dispenser tip with a notch or slant for directing the helical wire structure around a corner. FIG. 18-B and FIG. 18-C show the bunching anchor forming.

FIG. 19-A, FIG. 19-B and FIG. 19-C show mechanical stabilizers or insulators drawn in differing patterns on an actual helical wire structure.

FIG. 20 is an image of a mechanical stabilizer or insulator as drawn in FIG. OA.

FIG. 21-A, FIG. 21-B and FIG. 21-C show initial manufacturing steps for the wire rope.

FIG. 22 is a schematic of mandrels crossed for skeining with adjustable pegs for varying the angle of skeining.

FIG. 23-A, FIG. 23-B, FIG. 23-C and FIG. 23-D show steps of removing an electrode with the helical wire structure from a rodent.

FIGS. 24-A through 24-E are images of a sequence of complete removal of one embodiment of the electrode with a helical wire structure from a rodent's brachial plexus implanted for eight weeks, with post-mortem removal.

FIGS. 25-A through 25-D are a sequence of four images showing removal from a live rodent after a four week implantation by pulling from a middle portion, not the end, of a helical wire structure electrode, and showing unzipping on either side of the forceps grip.

FIGS. 26-A through 26-D are a sequence of four frames of the same procedure in FIG. 25 showing pulling the electrode from the middle not from the end, and showing unzipping on either side of the forceps grip.

FIG. 27 is an image of a helical wire structure, comprising a wire rope of 100 strands of 25 micron diameter gold wire, being pulled from the right side. Distance between coils increases, outer diameter decreases, from right to left.

FIG. 28 contains three images of one embodiment of the same helical wire structure electrode after having been pulled in three stages.

FIG. 29 is an image (50×) of the helical wire structure having been pulled as in a removal.

FIG. 30 is an image (100×) of a substantially linear portion of an electrode with tissue in-growth, comprising 100 strands of 25 micron diameter gold wire with an outer diameter of 0.75 mm, having been pulled and removed from a laboratory animal after implantation on a brachial plexus for 60 days.

FIG. 31 contains six images of unzipping of a bunching anchor and a substantially linear portion of an electrode.

FIG. 32 is a flow chart showing steps for removal in a clinical setting.

FIG. 33 is an experimental set up of wire ropes and helical wire structures of equivalent size injected into ballistics gel.

FIG. 34 depicts data comparing minimum force required to pull the wire ropes in FIG. 33 from the ballistics gel.

FIGS. 35-A through 35-H are a sequence of eight frames showing removal by super-twisting with an automated tool.

FIG. 36-A shows a portion of an electrode comprising a helical wire structure and stainless steel wire lead connected and surrounded by heat shrink polymer. FIG. 36-B shows, after injection into ballistics gel, the helical wire structure has bunched but remains connected to the stainless steel lead.

FIG. 37-A to FIG. 37-E show a sequence of injection of the helical wire structure electrode in FIG. 36-A and FIG. 36-B.

FIG. 38 is a diagram showing an electrode comprising a stainless steel lead in the center connected on each end to a helical wire structure.

FIG. 39 is an image showing tissue heating patterns using the present invention.

FIG. 40 is a schematic drawing of a helical wire structure electrode with bunching anchors connected by intermittent macro-coated helical wire structure and individually micro-coated wires. All of the details of the electrode are not shown.

FIG. 41 is diagram showing an electrode comprised entirely of a helical wire structure, with a central region insulated and the two ends uninsulated.

FIG. 42 shows a multi-polar electrode comprising a helical wire structure comprising 3 wire ropes (#1, #2, #3).

FIGS. 44-A, 44-B and 44-C are images of the tip of a double barrel needle and how the helical wire structure electrode is loaded into and then pushed from both barrels.

FIGS. 45-A through 45-F are a sequence of images showing injection of an electrode comprising a wire rope having hollow core and outer diameter approximately, from a double barrel needle into ballistics gel.

FIG. 46 illustrates a top view of surgical blades modified to include a hook, according to aspects of the present disclosure.

FIG. 47 illustrates a prototype example of a hook blade for electrode removal, according to aspects of the present disclosure.

FIG. 48 illustrates a detailed technical drawing view of a hook blade for electrode removal, according to aspects of the present disclosure.

FIG. 49 illustrates a side view of a delivery system for a split contact injectable electrode, according to aspects of the present disclosure.

FIG. 50 illustrates a side view of a split dual contact injectable electrode, according to aspects of the present disclosure.

FIG. 51 illustrates a side view of a standard and injectable electrode hybrid configuration, according to aspects of the present disclosure.

FIG. 52 illustrates a manufacturing sequence for a loopback electrode configuration, according to aspects of the present disclosure.

FIG. 53 illustrates a procedure for adding a crimp connection to a previously implanted electrode, according to aspects of the present disclosure.

FIG. 54 illustrates a cross-section of a connection configuration before and after crimping, according to aspects of the present disclosure.

FIG. 55 illustrates discrete components of a crimp and swage based connection system, according to aspects of the present disclosure.

FIG. 56 illustrates an orthogonal view of a multiple contact helical wire structure electrode, according to aspects of the present disclosure.

FIG. 57 illustrates an implantable generator connected to a dual contact electrode, according to aspects of the present disclosure.

FIG. 58 illustrates a perspective view of a percutaneous injectable electrode system, according to aspects of the present disclosure.

FIG. 59 illustrates a perspective view of a transcutaneously coupled injectable electrode system, according to aspects of the present disclosure.

FIG. 60 illustrates a perspective view of a multi-contact connector assembly, according to aspects of the present disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The present disclosure relates to helical wire structure electrodes, connector systems, and related devices for electrical stimulation applications. The disclosure encompasses several broad categories of embodiments including multi-contact helical wire structure electrode configurations, loopback electrode designs, connector systems for interfacing helical wire structure electrodes with pulse generators, interfacing solutions for transcutaneous and percutaneous coupling, and devices for percutaneous removal of injectable electrodes. The devices and systems described herein include both helical coils and non-helical coils and structures that carry an electrical signal from a proximal end to a distal end, wherein the proximal end is in communication with an electrical current generator or voltage generator and the distal end is implanted in a body of a patient or animal. The devices described herein are self-anchoring, and when inserted into the body the proximal end does not protrude, making the device less obtrusive to the patient. The time of deployment of the electrode is between 5 and 30 minutes, wherein a preferred deployment time is 15 minutes.

The current disclosure provides a clinician the ability to insert the electrode rapidly without surgery and supervision from support personnel such as manufactures representatives or medical support staff.

The current disclosure promotes reducing the time needed to insert the electrode and reduces the clinical logistics needed to complete the process or procedure. The process is less invasive and reduces complications. The process does not require sutures. The time of deployment is between 5 and 30 minutes wherein the preferred time is 15 minutes. The less intervention required promotes greater compliance and a shorter learning curve for the clinician.

The device of the current disclosure is self-anchoring and if inserted into the body the proximal end does not stick out making it less obtrusive to the patient.

The device of the current disclosure includes both helical coils and non-helical coils and structures.

The injectable electrode in the present application presents significant improvement over prior electrodes in several ways including without limitation novel changes in the anchoring and removal characteristics for implants for neuromodulation including stimulation and block, and also ablation and any transmission of energy in a body.

Securely anchoring the lead at the target tissue is a major need not fully solved by prior art devices. Migration of a neuromodulation lead away from its target has been shown to be by far the leading device-related adverse event in at least two recent independent studies of spinal cord stimulation (SCS), dorsal root ganglion stimulation (DRG), occipital nerve stimulation (ONS), sacral nerve stimulation (SNS) and peripheral nerve field stimulation (PNFS). Lead migration made up 59% of device-related adverse events, and 27% of the adverse events as a whole in DRG. Sivanesan, E., et al., Retrospective analysis of complications associated with dorsal root ganglion stimulation for pain relief in the FDA MAUDE database, Reg. Anesth. Pain Med. 44 (1): 100-106 (2019); Eldabe, S., et al., Complications of Spinal Cord Stimulation and Peripheral Nerve Stimulation Techniques: A Review of the Literature, Pain Med. 17 (2): 325-36, (2016). In DRG, lead migration made up 59% of device-related adverse events, and 27% of the adverse events as a whole. Sivanesan, E., et al., Peripheral lead migrations have been reported at rates as high as 100% in a case series of ONS at 3 years and 60% at the end of 1 year and in 12 of 51 subjects (24%) in the ONSTIM study of ONS. Eldabe, supra at 326. SCS leads were found to migrate in different studies ranging from 13.2% to 27% of patients. Eldabe, supra at 326. SNS leads were found to have migrated in 16% of patients despite the use of tined leads. Eldabe, supra at 327.

Explant and revision are also major aspects of reported adverse events for DRG implants. Of the adverse events reported to FDA, 49% required at least one revision surgery and 16% required explant surgery. Each revision or explant surgery presents new infection risk.

Explant surgery was associated with 83% of deep infections, and revision surgery with 9%. Severe neurological complications were associated with explant or revision surgery in 1.8% and 1.6% respectively in patients with reported adverse events. Difficult removal was reported 6.2% of the explant surgeries. “Difficult lead removal often prompted purposeful lead cutting, whole lead retention, or inadvertent lead damage, which resulted in a retained lead segment within the epidural space. Similarly, damage to the sheath used for lead placement sometimes resulted in a retained segment.” Sivanesen, supra, at 6.

A recent review of medical device reports (MDRs) in SCS by the Food & Drug Administration (FDA) found a lower percentage of specific lead migrations, 7.2%, but the leading patient problem code was inadequate pain relief, 28.1%. https://www.fda.gov/medical-devices/letters-health-care-providers/conduct-trial-stimulation-period-implanting-spinal-cord-stimulator-ses-letter-health-care-providers In this regard, it is relevant that lead migration of only 1 mm away from the tissue target can require a dramatic increase in current output by the lead to stimulate or block the target as the second spatial differential equation for the activation for neural tissue describes. In such a circumstance, failure to increase the current reduces stimulation or block, but increasing the current too high can cause the patient great discomfort and lead to nonuse.

There have been various approaches toward anchoring the lead in place. The manufacturer of a DRG product recommends traditional anchoring (tines) and strain relief loops. These measures, however, require access to the site by the physician, which in turn leads to some trauma as a result of the need for space for surgical implements necessary for the suturing to be performed. “[L]ead migration remains the most common complication of spinal and peripheral nerve stimulation. Although paraesthesia coverage loss due to lead migration can be recaptured by reprogramming, the majority of the instances of major lead migration require minor reoperation to relocate the lead to its original position and most will incur the cost of a new lead.” Eldabe, supra, at 327. Each revision procedure presents new injection risk, and the direct and indirect costs of the foregoing complications can mount quickly for patients, payors and society as a whole.

A better solution for removal is greatly needed. Previous particle-based versions of an injectable electrode present challenges for removal of 100% of the material injected. Some previous wire-based injectables have been conducive to in-growth by body tissues but can allow too much in-growth, without a ready means for severing the in-growth, requires levels of force for removal leading to excessive trauma, bleeding and pain to the patient.

### The Helical Wire Structure Electrode

More particularly, disclosed herein is an electrode comprising a helical wire structure which solves several problems with the prior art including without limitation suture-less anchoring on or near a tissue target and removal, both anchoring and removal being minimally invasive.

The present invention includes a helical wire structure electrode which can be injected into a body near or on a tissue target such as a peripheral nerve, nerve ganglion or a tumor and be anchored without sutures. Although the helical wire structure electrode includes at least one wire rope as an intermediate stage, the wire rope by itself does not have the properties of the present invention.

### Aspects of the Helical Wire Structure Electrode

An electrode comprising a helical wire structure as shown in FIG. 1 solves the very significant concerns with the prior art in a number of ways. The electrode 1 comprises a primary wire rope 22 comprising multiple parallel wire strands 4, with the wire rope being wrapped helically around a core to form a secondary wire structure, i.e., a helix comprising coils, which is flexible, bendable, stretchable, compressible and pushable.

As used herein, “strand” means a length of metal wire (or other material) which may be separate from all other strands or may be part of a continuous length. If a continuous length, the ends of the wire rope and the resulting helix have no sharp ends. A continuous length must break in at least two nearby places before pieces of wire are at risk of being left inside the body if the overall structure is removed. Breaking in two places near enough for a piece to be severed from the loop or, if severed, to not be brought along during removal by being wedged into the helical structure.

As used herein, “dispenser” means any one of a needle, cannula, catheter, tube, insert.

As used herein, “self-bending” includes that the helical wire structure has sufficient flexibility, when it meets mechanical resistance, to bend in any direction and up to 180 degrees on its longitudinal axis, without a clinician using a specific tool to bend it.

As used herein, “self-anchoring” means that the electrode needs neither sutures or other traditional anchoring techniques nor an open cut-down, or even laparoscopy, to secure the helix.

For a helical wire structure comprising individual lengths of wire twisted together, the wire ends may be crimped so that they do not irritate surrounding tissue, or they may be gathered and coated in a polymer on the circumference of the end but leaving the wire ends exposed to conduct energy. Individual wire ends may also be heated so they form a mass.

The electrode herein possesses a general shape and pattern of a helix made of at least one wire rope which comprises a number of strands. Although the general shape and pattern of the helix is predictable among all embodiments given standardized manufacturing techniques, the placement of an individual strand and the actual shape and dimension of a single coil in the helix may be random or irregular, but only in this sense within a general shape or pattern. In the same way, a bunching anchor from a helical structure with a given wire composition and standardized manufacturing may be random or irregular, but only in this sense within a general shape and pattern.

The helical wire structure electrode may comprise gold wire as well as other conductive wires selected from the group consisting of gold, silver, platinum, stainless steel, titanium, titanium-nickel, iridium, platinum-iridium, tungsten, platinum-tungsten and other metal alloys such as MP35N, a cobalt-nickel-chromium alloy with molybdenum added for corrosion resistance. Wires comprising the above metals are readily available commercially in the 2-300 micron diameter range, and wires of other diameters are also suitable for some embodiments of the present invention. The final selection of material and diameter for a particular embodiment is dependent on patient biocompatibility and desired tensile (mechanical) and electrical properties for the particular application and embodiment, as well as dependent on optimum force supplied by the wire structure electrode onto the tissues against which it is pressed, because mechanical forces from any implanted electrode influence formation of encapsulation tissue. Wires of these biocompatible metals have different mechanical and electrical properties, such as conductivity, and the potential effects for heating of the wires during the conduction of electrical current. The metal composition of the wires can be varied to introduce desired physical properties. The deployment process partially unwinds some of the strands 4 from the main helical structure and pushes them between 1 to 200 microns away that is mechanically distant enough from the helical structure 1 to be mechanically free to move with the tissue and have less encapsulation between that strand and the native tissue. Also, the strands which partially unwind are very flexible (because they are so thin and can move even more easily than a coil of the helical structure) and can be closer to the tissue target and help with conduction of energy. In this way the deployment process partially unfolds the tightly wound wire rope 22 forming the helical structure and helps to create more flexibility and thus better mechanical matching between the helix and the surrounding tissue. This is a helical macrostructure with a multi-strand microstructure.

FIG. 1 shows a gold wire rope 22 (pre-helical structure) at the top and an electrode with the helical wire structure 1 (lower). In the embodiment pictured here, the wire rope 22 of approximate 11 cm length results in a helical wire structure 1 of approximately 3 cm length as positioned on a guidewire 2 on which the helix was made. The ends 3 of the helical wire structure are round as this embodiment is made of strands formed from one continuous length of gold wire. Multiple wire ropes may comprise the helical structure, depending on the diameter of wire used for the wire ropes, and the desired flexibility of the helical structure.

When the guidewire 2 is removed from the electrode, there is a hollow core 5 surrounded by the inner diameter 17 of the helix 1 which allows for liquid, gel or gas delivery/transport through the helical structure while still inside the dispenser 10. This ability to transfer liquid, gel or gas into the body or out of the body (i.e., injection or suction) may be used during hydro dissection and formation or widening of the void 12 within or nearby the interfacing tissue target right before or during the deployment into the body of the helical wire structure.

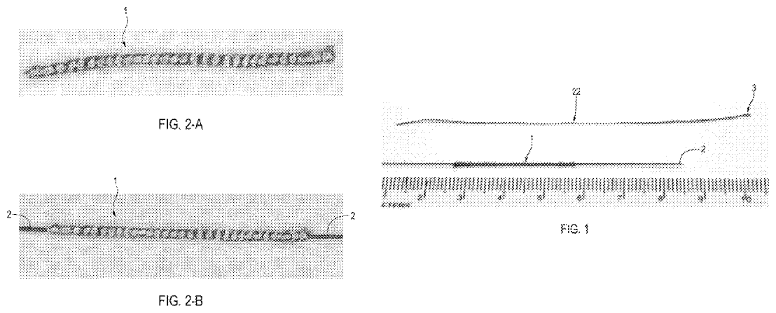

FIG. 2-A is an image (15.6×) of one embodiment of the electrode with the helical wire structure ex vivo after removal of a guidewire, and FIG. 2-B is the same structure (7.4×) with the guidewire 2. The wire rope is 100 strands of 25 diameter micron gold wire and is wrapped around a 0.25 mm guidewire with an approximate outer diameter of 0.75 mm. Overall length is approximately 2 cm and is made from 6 meters of continuous gold wire.

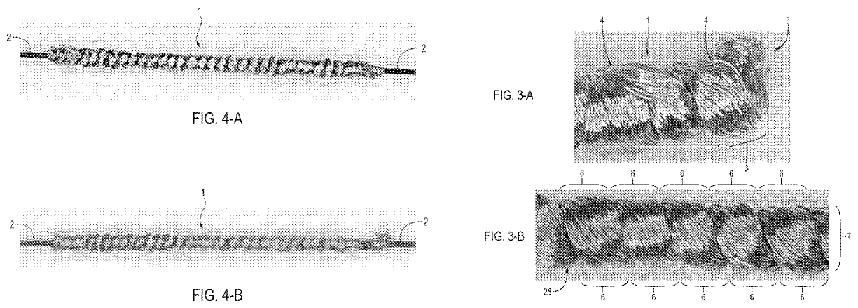

FIG. 3-A is a close up of an end of the electrode in FIGS. 2-A and 2-B showing an end 3, here rounded because the wire in the wire rope is continuous resulting in no sharp ends. FIG. 3-B is a close up (100×) of a middle portion of the electrode in FIG. 2-A and FIG. 2-B, and shows five full coils 6 (or turns) with two additional coils partially cropped out of the image. The outer diameter 7 of the electrode's helical wire structure is also shown.

FIG. 4-A is an image (14.3×) of one embodiment of the electrode with the helical wire structure 1 comprising 50 strands of 25 micron diameter gold wire and 5 wires of 75 micron diameter gold wire. FIG. 4-B is an embodiment with the helical wire structure comprising 10 strands of 75 micron diameter gold wire.

FIG. 5-A is a closer view (100×) of the electrode 1 with different wire diameters in FIG. 4-A, and the coil where the arrow is pointing has a different mix of wire diameters than those to the left of it. FIG. 5-B is a closer view (100×) of the electrode with wire strands of 75 micron diameter in FIG. 4-B. The average outer diameter 7 for the electrodes in FIGS. 5-A and 5-B are the same.

FIG. 6-A is an image of an embodiment of the electrode 1 comprising 100 strands of 25 micron diameter gold wire, each with a guidewire 2 of 0.45 mm diameter, and 6-B is on a guidewire 2 of 0.8 mm.

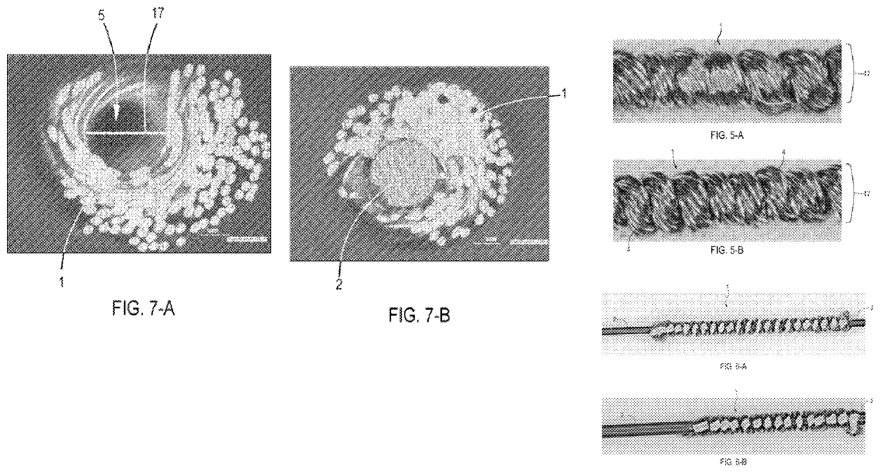

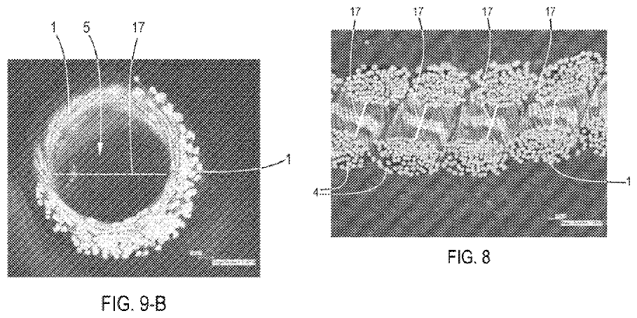

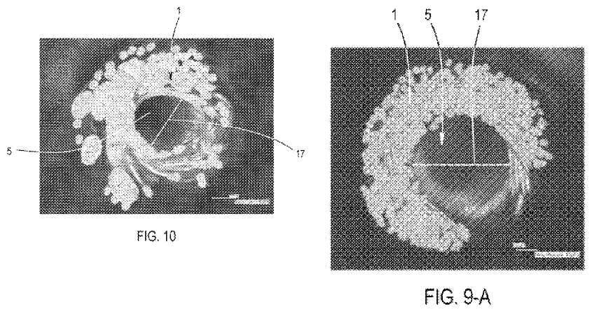

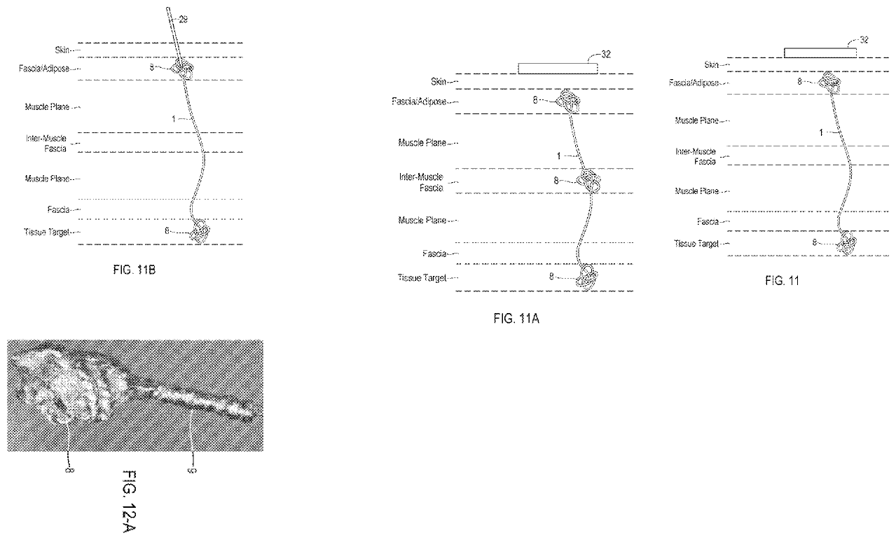

FIG. 7-A is an image (300×) of a latitudinal cross-section of an embodiment of the present invention electrode comprising a helical wire structure 1 comprising a wire rope comprising 100 strands of 25 micron diameter gold wire. Some distortion of the structure exists because of the cutting process. This figure shows the hollow core 5 defined by the inner diameter 17, here approximately 0.25 mm. FIG. 7-B is an image of the same electrode before the 0.25 mm guidewire 2 was removed. The guidewire was chipped slightly on the lower left by the cutting process. FIG. 8 is an image (150×) of a longitudinal cross section of the same electrode as in FIG. 7-A showing the inner diameter 17 defining the hollow core 5. Again, here, there is some distortion resulting from cutting, as in all of the cross-section figures herein. FIG. 9-A is an image (200×) of an electrode similar to FIG. 7-A and FIGS. 7-B and 8 but manufactured on a larger guidewire, 0.45 mm, and 9-B on a guidewire of 0.8 mm. In FIGS. 7-A, 8, 9-A and 9-B the guidewires have been removed. FIG. 10 is an image (300×) of a latitudinal cross-section of an electrode comprising a helical wire structure comprising a wire rope comprising 50 strands of 25 micron diameter gold wire and 5 strands of 75 micron diameter gold wire, with a hollow core 5.

FIG. 11 is a conceptual diagram of the helical wire structure electrode 1 as implanted with a bunching anchor 8 at a tissue target such as a nerve, with a second bunching anchor 8 in the subcutaneous region, which is able to receive transcutaneous transmission of electrical current from a TENS unit 32 on the skin surface. The bunching anchor serves as a connector pad to enhance reception of energy either transcutaneously from a probe 29 or from a TENS unit or similar. This figure does not attempt to show the helical structure or the exact nature of the bunching anchor 8, but just the general positioning of the helical wire structure within the tissue. The connector pad is the same a bunching anchor 8 shown throughout this specification which is close enough to the surface of the body to enable collection of electric current transcutaneously from a TENS or percutaneously by a probe 29 transmitting energy (the latter as shown in FIG. 11B).

FIG. 11A is the same as FIG. 11, except that an additional bunching anchor has been deposited in the inter-muscle fascia tissue layer. This demonstrates that multiple bunching anchors may be created to create unique and individualized anchoring for a particular application.

FIG. 11B is the same as FIG. 11, except that energy is being delivered by a probe 29 to the bunching anchor 8 serving as a connector pad.

Wire strand 4 diameters of a wide range of sizes, preferably from 12 to 250 μm, may be used to create the helical wire structure to allow different stiffnesses and spring characteristics of the helical wire structure, although a range of diameters down to 1 micron can be suitable. In some preferred embodiments, the electrode with helical wire structure comprises wire ropes comprising 25 micron diameter gold wire. In one embodiment, using 99.99% gold wire with a 25 micron diameter allows optimal mechanical and bio-inert characteristics for the biological tissue.

The diameter of the wire strand may be varied to create helical wire structures suitable for different implant locations. Helical wire structures may use smaller diameter wire for relatively small nerves of about 1 mm diameter. Larger diameter wire threads may be used more preferentially to manufacture helical wire structure electrodes for nerves of larger diameters of e.g., 2 to 5 mm such as the sciatic nerve that has a flat cross section width of about one inch (~25 mm) along its thickest axis, or even larger nerve cross sections. Thicker diameter wires may be used preferentially to support more tensile strength of the overall structure.

Images of some embodiments of the invention herein include FIGS. 1 through 6 as they exist ex vivo. In one embodiment the electrode comprises a helical wire structure comprising at least one wire rope 22. The helical structure is flexible and able to stretch or compress along its own longitudinal axis. This allows the electrode structure to remain mechanically intact and electrically conductive when subjected to push and pull forces from any direction. The helical structure is capable of bending easily at a small or a severe angle to its own longitudinal axis. This allows the structure to flexibly form and fill spaces by bunching.

As shown in FIG. 3-A, FIG. 5-B, and FIG. 8, for example, the strands on the outside of the helical wire structure allow for a soft integration with the surrounding tissues allowing for a pliable interfacing of the overall structure with the tissue. Also, without a guidewire, because the inner diameter 17 of the helical wire structure defines a core 5 is hollow and, to the extent the individual coils 6 are not bonded together, the coils can move in relation to one another by bending, stretching and compressing. The ability to “give” when the body moves reduces compression and inflammation of the tissue, resulting in a thinner encapsulation than for traditional neuromodulation devices with a single wire or a metal plate or large volume metal surface pressing against tissue. A thinner encapsulation response affords better electrical coupling (lower impedance) interfacing. The body's encapsulation of the invention herein is less than 100 microns, compared to encapsulation of prior art devices in a range up to 1000 microns. The stiffness of prior art electrodes, which are stiff in all dimensions, is a significant contributor to generating a thick capsule around it. The thicker the capsule, the less energy can pass through it.

The helical structure allows for in-growth of tissue including collagen and vessels to partially penetrate between strands, anchoring the coil without the need to suture the overall structure down to the target tissue. Natural integration with the surrounding tissue aids the overall capability of minimally invasive delivery via a thin (e.g., 18 gauge) needle because no sutures are needed. The mechanical integration caused by in-growth of tissues and the encapsulation of the electrode results in anchoring to the outer surface of the structure. The wire rope itself is not nearly as penetrable by tissue in-growth as is the helical structure which allows movement between coils and has a hollow core.

The helical structure is highly flexible, configured to fold along its longitudinal axis by 10 degrees, 30 degrees, or even 180 degrees in any direction (that is, back on its own original axis) as measured from its longitudinal axis. As the clinician is viewing the dispenser tip arrive at the tissue target (e.g., through fluoroscopy or ultrasound), she may hold the tip substantially stationary and begin pushing the electrode from the tip. The surrounding tissue resists the emerging electrode and deflects its path so that its flexibility causes it to bunch near the tip. Bunching during injection is an intentional characteristic designed to allow the clinician to optimally fill a void 12 (either created or naturally occurring) inside the body, either inside or near to the target tissue or structure of interest. The bunched macro-structure of meandering twists and loops creates compression against the tissue, or fills a created void, so that the bunching is larger in diameter than the outer diameter of the dispenser which has generated an insertion channel 14. That is, the deposit of a width of bunched coils of the helical wire structure wider than the dispenser and insertion channel, creates a mechanical impediment which keeps it in place during normal bodily movements.

Bunching of the helical wire structure allows for the custom design of the electrode by the clinician using an electrically insulated dispenser while in electrical contact with the target tissue during the insertion. This includes a smaller or larger bunching anchor for mechanical anchoring but also more or less electrically active surface area on or near a tissue target. The bunching anchor is enlarged or reduced in size during insertion from an electrically insulated dispenser by pushing it out and retracting it back into the needle and using the dispenser to provide insulation during the testing of stimulation, block, ablation or similar with the insulated/insulating needle still present. This provides the clinician with a very versatile tool while visualizing the injection dispenser itself as a part of the stimulation, block, ablation or similar system and let the bunching anchor form in front of the tip of the dispenser. Once the customized shape and size has been deployed and electrically verified to be optimal, insulator or mechanical glue may be deployed from the dispenser to insulate around the substantially linear portion of the helical wire structure leaving only the bunching anchor to electrically interface with the tissue.

During insertion, when the clinician begins withdrawing the tip, with the bunching anchor 8 holding in place, the portion of the electrode within the dispenser 10 exits from the tip to deposit a substantially linear portion 9 of the electrode, as shown in FIG. 12-A.

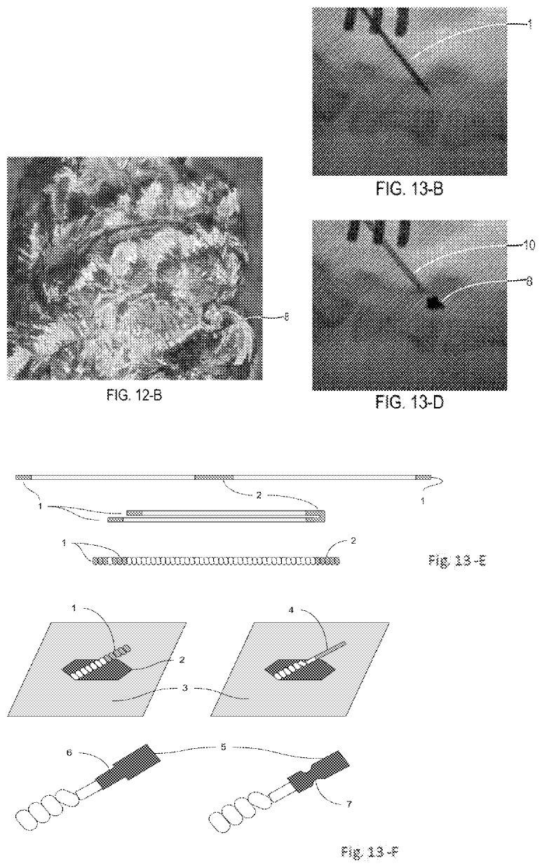

FIG. 12-A illustrates the bunching anchor 8 integral to a substantially linear portion of the electrode injected into ballistics gel. This figure is an image (20×) of one embodiment of the electrode, comprising 100 strands of 25 micron diameter gold wire with an outer diameter of 0.75 mm, injected and formed as a bunching anchor 8 into medical gelatin (00 hardness), with the bunched portion 8 integral with a substantially linear portion 9 deposited by movement of the tip 11 away from the bunching anchor. FIG. 12-B is a close up image (80×) of part of the bunched anchoring 8 of FIG. 12-A. FIG. 13-A is a fluoroscopic image of insertion of a dispenser 10 (needle) to a feline DRG. Note that the electrode 1 is not yet shown in the dispenser 10, but in FIG. 13-B the electrode 1 has been pushed near the tip 11. FIG. 13-C shows the electrode beginning to form a bunching anchor 8, and FIG. 13-D shows the bunching anchor 8 fully formed which is much wider than the dispenser. Because the insertion channel 14 through the tissue is the size of the outer diameter of the dispenser, the bunching anchor is held securely in place.

As used herein, “substantially linear” means either straight, somewhat wavy or gently meandering, but not a sharp bend, corner or a bunching.

Bunching of the helical wire structure prevents pistoning, a phenomenon in which a round or smooth structure, say a traditional or conventional lead wire, is able to move back and forth in a fibrous capsule. Bunching anchors, as described herein, may have a general shape which is consistent with the composition of the wire rope and the type of void, but bunching anchors are not a single, exact shape. Rather the exact shape of a specific bunching anchor results from several factors including the diameter(s), number and material of the strands, the tightness of the twisting of the wire rope, and the particular shape and character of the void into which they are injected. All of these properties of bunching anchors, and the helical macrostructure with a multi-strand microstructure (discussed herein elsewhere), contribute to resistance to pistoning.

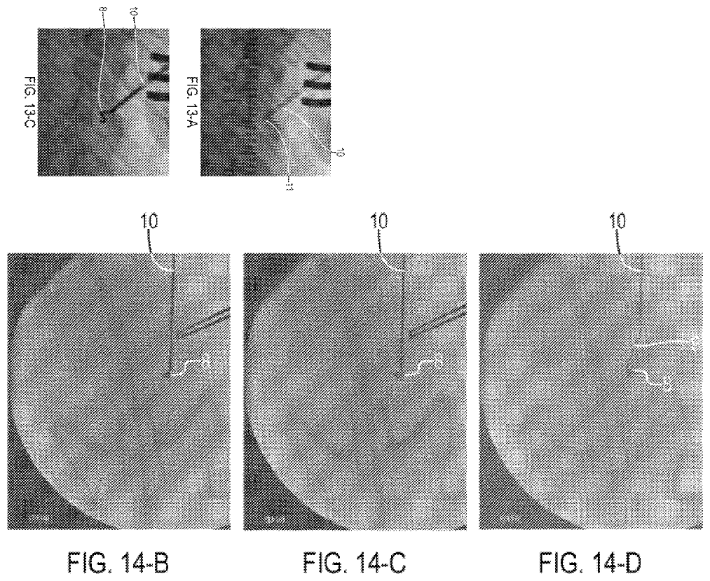

FIG. 14 contains fluoroscopy images showing another sequence of injection of the electrode comprising a helical wire structure. FIG. 14-A shows the initial side movement of the helical wire structure from the tip of the dispenser which is an 18 gauge needle. FIG. 14-B shows initial bunching while the needle tip is substantially stationary, and FIG. 14-C shows additional bunching. FIG. 14-D shows that the bunching anchor 8 holds in place while next a substantially linear portion of the electrode 9 is deposited by withdrawal of the dispenser tip.

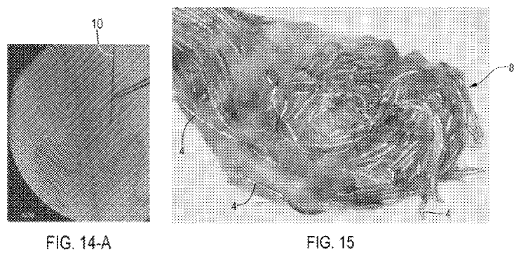

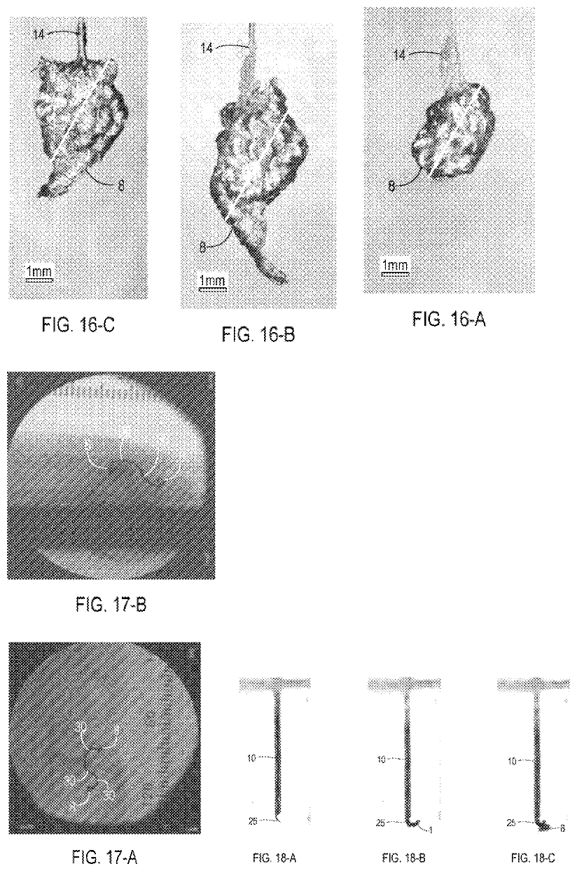

FIG. 15 is an image (200×) of a bunching anchor 8 of an electrode, comprising 100 strands of 25 micron diameter gold wire with an outer diameter 7 of 0.75 mm, after having been implanted on a brachial plexus for 60 days. The image shows tissue which has in-grown into the electrode. This bunching anchor was not unzipped, as discussed elsewhere herein, so it retains its bunched shape.

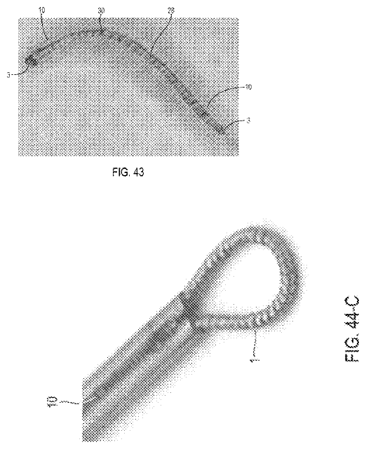

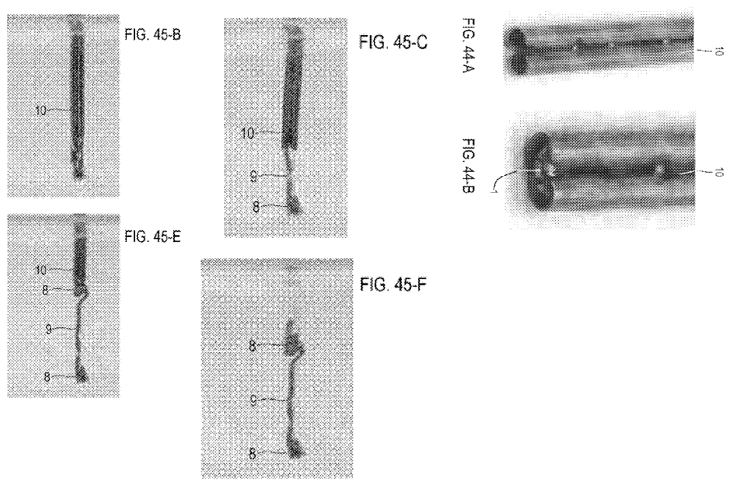

FIG. 16 shows that the bunching characteristic of the helical wire structure electrode varies when comprising wire ropes of different compositions. FIG. 16-A: 100 strand 25 micron, 3 cm length (9 cm rope), 0.25 mm core. FIG. 16-B: 50 strand 25 micron, 5 strand 75 micron, 3 cm length (9 cm rope), 0.25 mm core. FIG. 16-C: 10 strand 75 micron, 3 cm length (9 cm rope), 0.25 mm core. All three injections were made into medical gelatin (00) from 18 g cannula which was substantially stationary. White lines drawn in the same position show that the smaller diameter wire in FIG. 16-A produces a smaller bunching anchor 8. Varying the ratio of 25 to 75 micron wire produces a product that is either more flexible/compactable (16-A) or more rigid and less compactable (16-B and 16-C). Given a similar gel or tissue, smaller diameter wire yields a bunching smaller which is more compactable and thus smaller. Varying the wire diameter allows for the deterministic design of a bunching anchor volume prior to deployment by the clinician. The thicker diameter wires used in this example show the ability to create a bunching anchor of at least 5 mm in one dimension in 16-B and 16-C whereas the thinner diameter wire create smaller (3-4 mm) diameter bundles. All helical wire structures were deployed through the same inner diameter injection needle and all helical wire structures occupied the same length of needle inside the needle. The choice of wire diameter as well as the wire count, wire material and length of the wire inside the needle to be deployed from allow for a deterministic delivery of an anchor diameter without regard to clinician skill, especially from a multi-barrel dispenser as discussed herein. See, e.g., FIGS. 44 and 45 and related discussion.

FIG. 17-A is an image of an electrode with a curved portion within the connective tissue cavity around a rodent sciatic nerve. The curved portions 30 further assist anchoring along with the bunching anchors 8 at each end. The electrode is approximately 5 cm long, and comprises a wire rope of 100 strands of gold wire of 25 micron diameter, made from 15 m of wire. FIG. 17-B is the same implanted electrode from a different angle.

FIG. 18-A shows a dispenser tip with a notch or slant 25 for directing the helical wire structure 1 around a corner in ballistics gel. FIG. 18-B and FIG. 18-C show the bunching anchor 8 forming.

During implantation/injection, the helical wire structure may be super-twisted in the direction of its present helical direction of twist, or it may be untwisted in the opposite direction of the helical structure or super-twisting and untwisting the dispenser may be alternated to achieve an implanted electrode which is either more or less open to tissue in-growth, the latter of which assists with anchoring. This twisting and untwisting allows for a different tendency of the helix to fold, bend, roll, meander or compact on itself while being deployed from the dispenser, thus forming volume locations of different wire densities within the overall cavity.

As shown in FIGS. 12-18, the helical wire structure has sufficient mechanical strength to be deployed from a dispenser by pushing it out by a plunger, water pressure, or a hydrogel or other means of mechanical interaction so that it exits in a predictable manner into a tissue at a determined rate. Existing the dispenser tip, the helical wire structure starts to encounter opposing forces from bodily tissue preventing direct linear exit from the dispenser.

These opposing forces force the helical wire structure to fold up on its linear axial direction, causing a bunching (or bundling) of at least a portion of the helical wire structure electrode, while nestling tightly against and conforming to the target tissue. The bunching anchor 8 fills a void 12 which is not void of all matter, as there may be present liquids (e.g., interstitial fluids, a hydrogel) gasses (e.g., air, inert gas), but the void is not filled with solid tissue. The void may be a tissue plane or tissue void that is being created during or just prior to the deployment process of the helical wire structure by injecting e.g. a liquid through the hollow core of the helical wire structure in the as the manufactured helical wire structure may have a channel allowing the passage of a liquid (or a gas or a hydrogel, or another compressible or non-compressible volume) along the axis of the manufactured helical wire structure while it is still residing partially or fully within the dispenser. The helical wire structure enables the easy filling of the void within the tissue because the helical wire structure bunches upon itself, filling the void with what becomes a bunching anchor 8, a larger diameter of helical wire structure electrode than the outer diameter of the dispenser and the insertion channel 14. The void may be created by a blunt dissection during the deployment of the helical wire structure into the bodily tissue.

The bunching anchor 8 is thus configured to help create a void and also to take on the shape of an existing void by mechanically interacting with the tissue in which it is deployed. It may retain that shape by forming a negative imprint of the shape of the interfacing tissue, so that it assumes complex shapes inside or near the target tissue, partially or fully surrounding the target tissue for an optimal mechanical and electrical interfacing with a target tissue. It can fill a space inside a mechanically harder tissue (e.g., bone), a canal inside a bone, other more resilient body tissue if this void is being created during the deployment process within a naturally occurring soft-tissue filled volume inside the mechanically harder tissue. An example is a channel passing through a bone that is filled with nerve and fatty tissue. Another example is a nerve entering an organ, i.e., the innervation point of an organ, where a void may be formed just beneath the endothelial tissue surrounding the organ, thus allowing for the mechanical fixation of the helical wire structure inside the organ right beneath the endothelial tissue and near an innervation point. This helical wire structure can also fill a void within a sheath surrounding a nerve or other tissue target like an organ, allowing for the anchoring of the helical wire structure inside the target's sheath without the need to suture the helical wire structure to the nerve.

The bunching anchor 8 has the ability to fill a void volume (space) within the tissue to achieve a mechanical holding force sufficient to compress the helical wire structure electrode within the tissue and retain the shape of the filled void volume, so that the helical wire structure electrode takes up the contours of the void volume and retains these contours even if the tissue were to be removed. This effect is especially true after an additional period of tissue ingrowth into the helical wire structure electrode during the body's tissue foreign body response to the now placed helical wire structure electrode, commonly referred to as encapsulation. This means that the helical wire structure electrode does not require sutures for its mechanical stabilization to retain sufficient volume of the helical wire structure electrode near or at a target tissue location of choice by allowing the creation of a mechanical anchoring structure within the tissue of interest, near the tissue of interest to deploy energy into or remove energy from it. The helical wire structure electrode may be further mechanically stabilized by fibrous tissue during the encapsulation process of the body. The in-growth of biological tissues that are supported by blood vessels are able to mount a localized immune response if bacterial, viral, fungal or other biological or non-biological contaminants were to be introduced onto or into the chronically formed composite of helical wire structure base and in-grown biological tissue. The presence of blood vessels providing white blood cells and other elements of the body's immune system are helpful to provide a localized immune response that starts and likewise ends within the vicinity of the composite as well as to the immediate surroundings of the composite.

The bunching anchor allows for a deployment by needle without any penetrating elements of the wire structure present outside of the needle such as tines known in the prior art which would otherwise be required to form an anchor. This means the helical wire structure is self-anchoring because of the bunching anchor 8 which is larger than the insertion channel 14 through minimally invasive insertion into the target tissue. During the first two to four weeks (and beyond) after injection the bunching anchor 8 provides mechanical anchoring.

Tissue in-growth between and into the coils then begins to provide more anchoring. Then, loosening of strands from the main helical structure provides further ways for body tissue to fasten itself to these outlying pieces of the entire structure. All this prevents lead migration and eliminates the need for sutures.

The helical wire structure is immediately capable of being an anchor because, unlike a particle-based approach, it does not require curing in vivo.

The helical wire structure's flexibility allows for a deployment by a bent or curved dispenser into a shape that is not a straight line. The deployment into a non-straight line allows it to provide an energy concentration effect (“lens effect”) of energy delivered by the wire structure, thereby concentrating delivered energy into a target tissue.

As implanted, the helical wire structure electrodes cross tissue boundaries and resist shear forces more than non-helical structures because the former bend, stretch, compress and contract because they are hollow and comprise many wire strands which move in relation to one another. The present invention, when deployed into tissue planes, between tissues, into neural tissue or into a neural sheath, is mechanically adaptive with movement of the sheath with respect to the surrounding biological tissue. This ability to flex and bend (because of the movement of wire strands relative to one another) more readily than solid objects to resist breaking due to the inherent formation of a rope like structure, combined with the ability to fold in on itself, meander, fill an intentionally created void that is being filled in part with a meandering and folding helix to form an anchor inside a nerve sheath or at a neural innervation point to an organ allows for the creation of a neural interface by needle without the need to suture down the folded wire structure, thus a significantly less invasive delivery procedure, a significant advantage for the patient, the physician (time) and clinic (time per patient) and payer (cost per procedure).

The bending and meandering of the helical structure is visible on ultrasound and fluoroscopy, aiding the clinician with the ability to visualize the structure they are creating during the deployment process non-invasively.

The helical wire structure may be formed into a helix with a trailing (or front-running) tail of rope that is not fully wound into the helix.

The helical wire structure may be interfaced by pushing needles or probes into its overall structure, or into the rope forming the helical structure. The rope and the helix aid with supplying mechanical forces that press the single wire strands against the needles penetrating the helical structure or the rope, thereby increasing the reliability of the mechanical and the electrical connection. As a result of the bunching anchor's diameter being larger than the diameter of the helical wire structure (even more so than that of a wire rope), the bunching anchor 8 is easier for the clinician to locate and contact with a needle or probe transmitting energy to the electrode.

The helical wire structure is also capable of receiving energy preferably by the bunching anchor 8 collecting energy transmitted transcutaneously from a TENS unit 32 or other external device such as a dermal multiplexer.

The helical wire structure may be anchored into bony tissue optionally with the added delivery of bone cement or a tissue glue in the hollow core. The helical wire structure allows for a significant filling of a bony cavity with straight or meandering helical structure during the delivery process and subsequent (or parallel) filling of the cavity with bone cement or tissue glue. This aids with the formation of a mechanically stable and electrically close interface for neuromodulation.

The hollow core of the helix allows for the delivery of liquids (saline, lidocaine, steroid, bone cement, tissue glue) during the injection process through the same needle. This allows for the fewest needles to be used during the injection process in the clinic. It further allows for the deployment of the helix by one plunger while liquid is being pushed around the plunger into the dispenser, thus delivery of liquid and helical structure at different injection speeds. The helical wire structure allows for the adaptive filling of a void 12 created just prior to or during insertion of the helical wire structure, or one that occurs naturally. The insertion may include pushing a liquid (or liquids) quasi-simultaneously via the same dispenser that is used to inject the helical wire structure. The liquid being used may have a mechanical stabilization effect, an effect to limit or control future tissue ingrowth or an effect to electrically insulate the helical wire structure electrode at specific locations and distances on the entire length of the helical wire structure electrode.

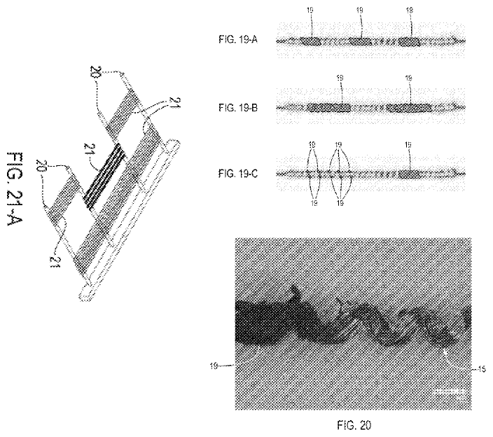

In FIG. 19-A, FIG. 19-B and FIG. 19-C, mechanical stabilizers or insulators 19 (e.g., a glue such as cyanoacrylate, a biocompatible paint or similar) are shown schematically as added to an actual helical structure at one location, at several locations, at specific intervals, or throughout the entire length of the helical structure to aid with the helical structure resisting deformation within the needle during the injection process and to aid with a select integration with the biological tissue post deployment. FIG. 20 is an image of such a mechanical stabilizer or insulator 19. The mechanical stabilizer or insulator will prevent or reduce cell ingrowth after implantation. This allows a controlled mechanical integration with the tissue, providing different areas of the electrode with the ability to integrate with the surrounding tissue variably, i.e., integration where there is no such mechanical stabilizer. This allows for handling differences in local shear forces as well as differences in forces needed to explant or move the overall helical structure after integration by the body's encapsulation process. Another mechanical stabilizer such as parylene C or similar polymer provides electrochemical passivation of the entire structure (or parts of the structure) or serves as an electrical insulator against the electrolytes in interstitial fluid. In contrast to mechanical stabilization agents listed earlier, parylene Cis in the range of single digit micrometer scale, thereby not limiting cell ingrowth into the overall structure of the helix or in-growth into the wire rope between strands on the single digit to small double digit micron scale. The parylene C (or similar) may be applied selectively with a pre-passivation by a cover agent prior to application of parylene C followed by stripping said pre-passivation agent; or the parylene C may be removed by an etch process at select locations (e.g., one end, both ends and where needed in the middle section of the helical wire structure) following the parylene C deposition. Lead integrated wire structures as described may be delivered onto multiple successive anatomical targets within one injection path and procedure. Said helical wire structure may further be placed on one nerve structure with a succession of interface points along the nerve to allow for a multi-polar interfacing of one nerve with the placement via one needle path. The helical wire structure may first be placed on anatomical target at two locations as shown (part A and part B), before the delivery needle is retracted and delivery of the distal parts as completed, subcutaneously as shown here. As shown herein, the multi-polar device may also comprise a continuous connection with multiple bare wire structure regions, as opposed to independently connected regions.

Instead of a single wire, a drawn filled tube (DFT) may be used to produce the initial spool of thin wire that will then be used to manufacture the parallel wire strands. The advantage of using DFTs is that they may have an outer interface metal (e.g., platinum) and an inner more electrically consecutive metal such as silver or gold to aid with the minimization of impedance across the long distance of the parallel wire strands inside the entire length of the rope making up the helix of the helical wire structure electrode.

Different densities and volumes of wire deployed at different locations throughout the cavity provide for different mechanical properties (higher stiffness of the final macro structure, or higher flexibility of the macro structure where needed) as well as different electrical properties (higher electrode to electrolyte interface area to allow more charge to be deployed at specific locations) as needed.

The many wire strands 4 in the wire rope ensure that electrical conductivity is maintained even if one or a group of the single wire strands were to be severed during the manufacturing process, the storage, the shipping, the deployment into the living body or during the duration of being located inside the body and potentially being subjected to stretch and shear forces within the body. In the example of 100 parallel strands of gold wire, losing the continuous connection of up to 50 of the wires would only slightly increase the overall impedance of the entire helical wire structure—but the severing of up to 50 of the strands is highly unlikely without being cut by a tool. The twisting of the parallel wire strands prior to helical winding ensures that there is a tight packaging of the overall wire per helical structure volume. Furthermore, if one or more of the wires were to be interrupted at specific locations then they remain attached to the entire structure, reducing the possibility of loose particles being separated from the structure as a single strand would need to break in two nearby locations to create one loose piece. A single break would not create a loose piece of strand, just two open ends which by their own nature are then even less likely to break off due to increased mechanical flexibility of the open ends when compared to the relatively higher stiffness of a wire loop prior to the loop breaking. If the rope were not composed of many (i.e., 5, 10, 50, 100, 200 or more) strands, but only one strand, then mechanical bending and shear forces would be more likely to cause a fatigue break that may result in loose pieces. The creation of the wire rope from numerous strands greatly reduces the risk of a fatigue break creating loose pieces, increasing the probability that the entire structure will be removed as a whole if undertaken.

### Manufacturing Methods

The following is one embodiment of a manufacturing method for the helical wire structure:
1. Wind metal wire at a set distance around 2 or more mandrels 20 to form 2 to 20,000 parallel strands.
2. Optional: add additional mandrels for skeining wire or other material of different diameter.
3. Remove parallel strands from mandrels and twist along the longitudinal axis to form a rope.
4. Optional step: apply mechanical stabilizer or insulator to rope at specific locations (such as a glue) and let dry to ensure the rope does not significantly unwind during the helix winding process.
5. Wind rope around a guidewire of outer diameter (preferably within a range of 0.1 to 2.0 mm) to form a helical wire structure around the guidewire with an outer diameter of the helix structure within a range of 0.2 to 3.0 mm.
6. Optional step: apply mechanical stabilizer or insulator to the helical wire structure electrode at specific locations (such as a glue) and let dry to ensure the rope does not unwind during the helical winding process. The guidewire may be coated with a non-stick substance (Teflon, or dissolvable paint) prior to application of the mechanical stabilizer or insulator to allow a follow-up step of releasing the mechanically stabilized or insulated helical structure from the guidewire mechanically such that the guidewire may move inside the longitudinal channel within the helical wire structure.

Steps 1 through 4 are performed on the rope manufacturing apparatus, i.e., mandrels, and steps 5 and 6 are performed on the helical winding manufacturing apparatus, e.g., winding the wire rope around a guidewire.

The mandrel-based manufacturing of the wire rope leads to ends 3 which are rounded when formed by continuous wires, thereby lessening the possible risk of sharp wire ends unfavorably damaging tissue or breaking off loose pieces. The mandrel-based manufacturing step of the initial rope leads to rope formed by 3-5 to 100 s of parallel strands of single wires which increases the reliability of the electrical conductivity of the helical wire structure. If, during the acute or chronic deployment of the helical wire structure, one or a few of the single wire strands were to break, then there is only an insignificant reduction in the overall ability of the helix to conduct electrical energy. The winding of parallel wires and helical twisting of the wire structure causes a higher compression ratio of wires to volume closer to the core of the structure, limiting the amount of cell ingrowth at the inside vs the amount of cell ingrowth and subsequent mechanical anchoring towards the outside of the wire rope comprising the larger helical structure.

The manufacturing apparatus for a wire rope of parallel wires with looped ends twisted to a precise diameter in one embodiment comprises: 1. At least two mandrels 2. Motor (with or without force measurement and/or torque measurement) 3. Velocity controller 4. Visual or electrical or mechanical sensors to detect continuity of the supplied wire 5. Wire Tensioner (with or without pins, guides, or clamps) 6. Wire Twisting Motor

The manufacturing apparatus for producing a helical wire structure electrode from at least one wire rope comprises a Guide Wire tensioner and holder: 1. Rope wire structure securing mechanism 2. Winding motor 3. Rope tensioning sensor and controller during winding

The product remains on the guide wire for transfer to a delivery cannula.

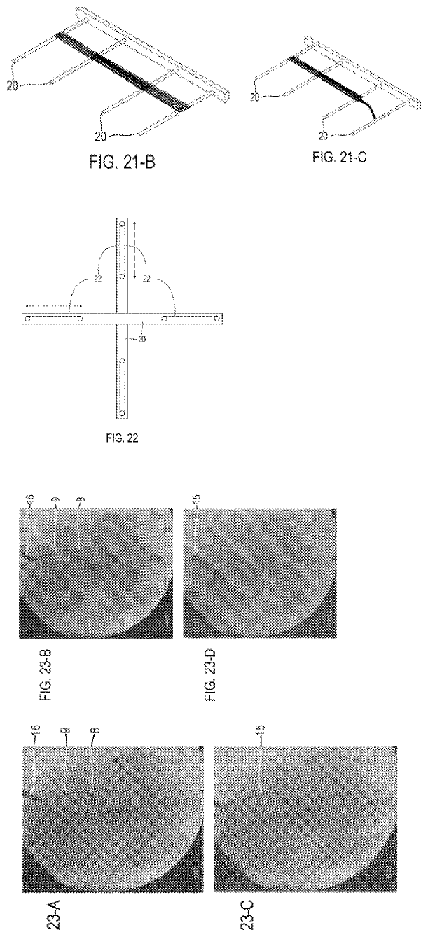

FIG. 21 shows one embodiment of initial manufacturing steps for the helical wire structure. FIG. 21-A shows wrapping different diameter wire or materials around mandrels into skeins with distinct profiles around 4 mandrels for creation of 3 distinct sections. Each skein may consist of different material, including nonconductive material (e.g., Prolene® suture, Vicryl® suture, silk or similar) to provide greater insulation or strength. FIG. 21-B shows merging the wire skeins with distinct profiles. FIG. 21-C shows the twisting of one of the distinct sections into a wire rope 22 which can be coiled into a helix or left uncoiled as a tail 18.

Instead of using only two mandrels that cause the wire from the spool to be pulled at significantly different speeds (and thus forces) during each turn, more rods may be used to have a more even pull speed and pulling forces. An example is to go to 3, 4, 5, 10 or more mandrels with an understanding that a larger number of mandrels at some point becomes a wheel like structure with spokes, as in FIG. 22, and the parallel wires wound around the number of mandrels or one wheel are more easily pulled off if there is a location to hold on to all the wires while taking them off the rods/wheel prior to the twisting step.

As shown in FIG. 22, a distance adjustment of the mandrels may aid with skein removal from multiple mandrels. FIG. 22 is a schematic of mandrels crossed for skeining with adjustable pegs for varying the angle of skeining.

Another method of manufacturing has all the elements above but adds electrical sensors to verify wire continuity at specific location or, optionally, visual sensors for verification of proper twisting outcomes, counters and torque measurement sensors that provide electrical feedback information to a computer controlled manufacturing process which uses the sensor information to determine the optimal manufacturing speed. Electrical sensors enable an easy measurement of the continuity of the wire strands at a high manufacturing speed at which the visual verification of <100 micron wires may not be as easily detected based on the resolution of the camera and processing (i.e., artificial intelligence) software.

Cleaning of the helical wire structure electrode occurs during several steps throughout the manufacturing process as well as within the supply chain. Components with direct human contact, at a minimum, undergo a cleaning operation prior to assembly into the final medical device. Manufacturing aids that are in direct contact with these components (such as mandrels and guides) are also cleaned on a regular basis to prevent transfer of any foreign materials to the finished device during manufacturing. Once wound into the helical wired electrode structure, the product undergoes another cleaning operation. Items used in the cleaning process include without limitation ultrasonic cleaning baths, isopropyl alcohol, water soluble detergent and mechanical cleaning apparatuses (e.g., brushes, wipes). Manufacturing of the helical wired electrode and delivery system occurs in an environmentally controlled area. Personnel are required to wear PPE (personal protective equipment) such as gloves and lab coats to prevent the introduction of foreign materials into the device or components. One of the benefits to the helical/mesh shape and structure in a cleaning operation is that it provides maximum surface area for cleaning agents and methods to contact the electrode effectively. The cleaning process for the helical wired electrode is detailed and recorded in manufacturing documentation and has been validated.

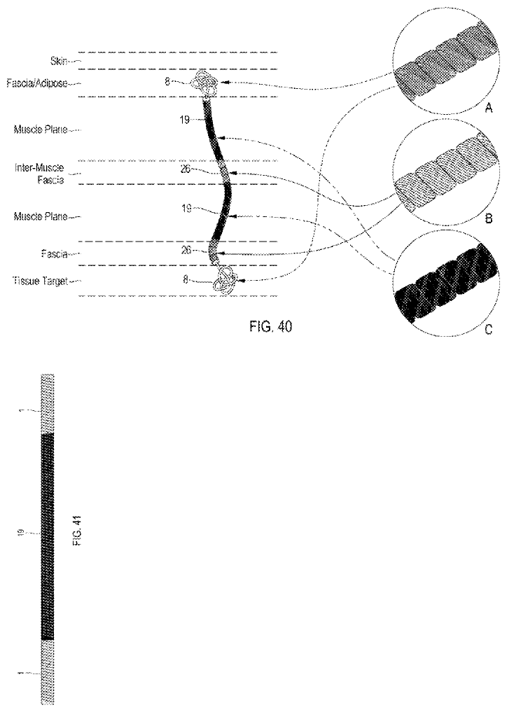

The micro coating depicted in FIG. 40 would be applied selectively while the wires are on the mandrel and skeined. Those skeined bundles can be separated so the individual wires do not touch one other. Then they are twisted together to form the wire rope with individual strands coated with an insulator. The macro coating is applied after the step of twisting of all strands to make the wire rope. The macro coating can be applied on top of the micro coating twisted sections of the rope also to make for better transitions from no coating to micro coating to macro coating. The micro coating can also be applied during the skeining process. As the wire comes off the spool, coating can be selectively applied—either via dip, spray, or extrusion.

### Minimally Invasive Removal

The present invention, when chronically implanted, allows for tissue to surround the wire structure on the outside perimeter, and likewise grow into the inside element(s) of it. The first approximately 1 to 5 cell layers in close proximity to the helical wire structure are an “inner layer” of encapsulation and the cell layers further away such as cell layers approximately 6 to 10 are an “outer layer.” While the count of cell layers forming the inner and outer layers may vary some, it is the differentiation of different layers that helps to visualize the concept of one layer having formed closer to the helical structure and another layer having formed closer to the native tissue. When the helical wire structure electrode is being removed from the living body then some inner cell layers may remain attached to the helical wire structure electrode while some of the outer cell layers may remain attached to the native tissue. When removal of the helical wire structure electrode from the living body is aided by the helical wire structure electrode helping to separate consistently and reliably an inner encapsulation layer from an outer encapsulation layer then the helical wire structure electrode has the in-built quality of easy removal as the pre-determined way of how an inner encapsulation layer separates from an outer encapsulation layer allows for consistency and predictability of an easy removal process for the clinician.

Prior art devices include platinum nerve cuffs which must be sized during implantation to minimize restriction of blood flow through the nerve. Over 60 mmHg pressure applied to a nerve will restrict blood flow down the line, due to a constricted blood vessel along nerve. Cuffs comprise a flexible silicone cuff and electrode contacts. If a nerve has a 2 mm diameter, fibrous encapsulation of the cuff will be at least 0.2 mm, and provides mechanical insulation from mechanical forces and, even when cuffs are flexible, a 0.8 mm cuff will be 1 mm around the outside of the cuff. Nerve cuff removal options in the status quo include: open cut-down surgery with dissection down to the nerve and the physical pulling of the cuff away from implant location with forceps. It is highly invasive and carries risk of infection and bleeding. Merely cutting the connection wire leaves a nerve cuff implanted in body.

Ability to remove the structure is aided by the mechanical stability of the structure and tear resistance based on the folding structure, size of core wire and number of wires used.

To facilitate minimally invasive removal of the present electrode, electrical, mechanical or thermal energy may be applied from the electrode into the surrounding tissue either to dislodge it from the surrounding tissue, or to ablate blood vessels or other tissues that have grown during the encapsulation process to surround or permeate the electrode. The minimally invasive removal process includes pulling on it with forceps 16 or similar, from any location including the middle portion 28 or the end 3 closest to the surface of the body, which causes the helical wire structure to contract on itself, tightening the roll concentrically along its longitudinal axis, thereby aiding with the dislodging from the surrounding tissue at a very local micron level scale, before starting to move along its longitudinal axis step by step as it is being removed. The analogy is to the teeth of a zipper, which are unzipped one at a time, and the unzipped teeth retain their structure though, in contrast to a zipper, the coils 6 of the helical wire structure electrode can change in size, shape and spacing during the pulling process. This means that the clinician is not pulling the entire helical structure, but only unzipping one coil at a time, thus reducing the force required to remove, as shown by the figures and data herein. Furthermore, the pulling process may represent a mechanical deformation of the helical wire structure electrode in a plastic, which is non-elastic, way that ensures that the clinician can easily determine if a helical wire structure electrode is in its original flexible state or after partial or full removal is then in a new stretched state from which it does not spring back into its original shape, ensuring that a stretched helical wire structure is not accidentally reimplanted.

Ease of removal of the helical wire structure is one of its great advantages. After applying local anesthetic, the physician makes a small incision on the skin above the uppermost portion of the electrode and pulls out the helical wire structure which unzips individual coils of the structure as they release from the tissue first pulling inward towards the center line of the helical wire structure electrode before being pulled outward to exit the body. When a coil 6 is pulled, the outer diameter contracts and pulls the outer surface away from surrounding tissue. This releases the inner encapsulation layers from the outer encapsulation layers one coil at a time, allowing the removal at low overall forces as one loop separates tissues mechanically prior to moving outward instead of prior electrodes and leads that require the attached encapsulation layers to remove along the entire implant at once. The separation from encapsulation lays one coil after the other ensures low removal forces and greatly adds to the reliability of the removal process that is easily performed in a clinic or outpatient procedure without major risk of the helical wire structure electrode rupturing, breaking (or “cutting” as clinicians refer to it) of a lead or electrode structure. The in-built quality to deform one coil at a time and thereby reduce the outer diameter of the helical wire structure electrode, while stretching the total length of the helical wire structure, swaps larger outer diameter for more length during the removal process, thereby reducing the removal forces, similar to how an inclined plane reduces the forces needed to lift an object one small incremental step at a time instead of lifting it all at once. This process of incremental removal of one coil at a time, herein described as “unzipping,” is of great benefit to the clinician conducting the removal process (and the patient) as unzipping reduces the possibility of loose pieces, aids with the release from the encapsulation tissue as well as delicate native tissues and enables exiting the body via its own path or footprint occupied chronically inside the body prior to removal.

FIG. 23-A shows a step of removing an electrode with the helical wire structure from a rodent (with forceps 16 attached near the end of the electrode nearest the skin) unzipping the substantially linear portion 9. The bunching anchor 8, opposite the forceps 16, is anchoring the electrode in a subcutaneous pocket. FIG. 23-B shows that the bunching anchor has begun to unfold because the linear portion 9 has already been pulled to its greatest length. FIG. 23-C and FIG. 23-D show continued pulling, with the bunching anchor completely unfolded.

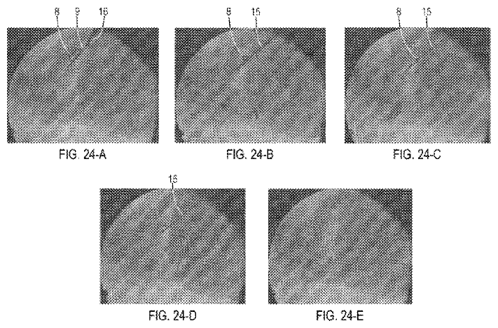

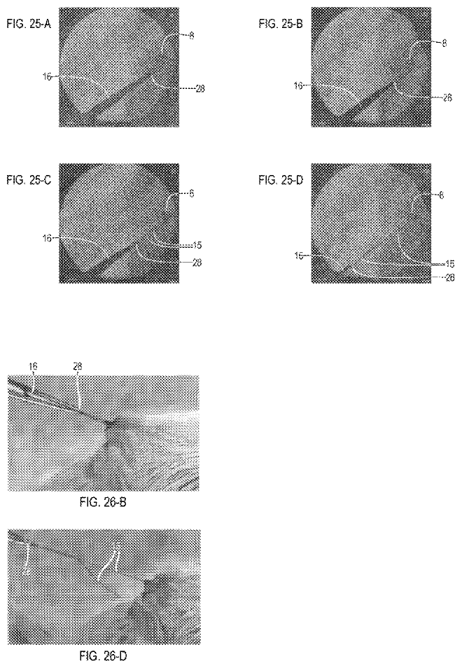

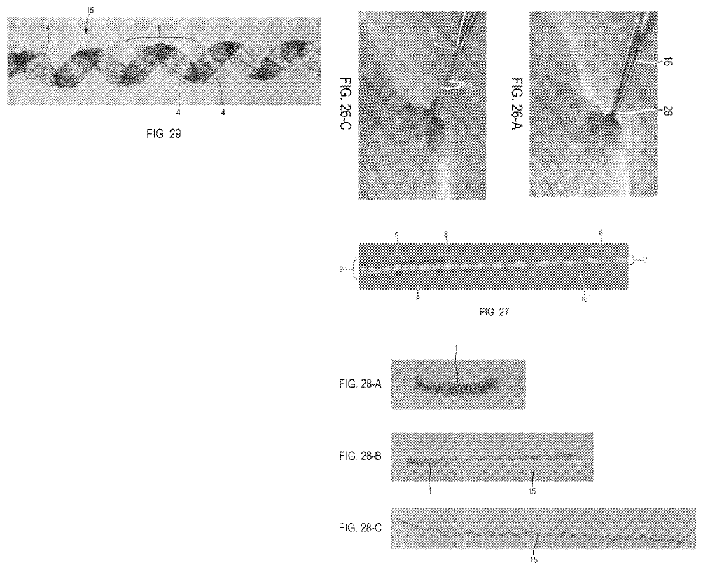

FIG. 24 shows a complete sequence of removal of one embodiment of the electrode with a helical wire structure from a rat's brachial plexus, implanted for eight weeks, with post-mortem removal. FIG. 24-A shows forceps beginning to pull an end at a linear portion near the bunching anchor 8. FIG. 24-B shows the unfolding of the bunching anchor as the linear portion 9 has been pulled to its greatest length and FIG. 24-C shows continued unfolding and an unzipped portion 15. FIG. 24-D shows that all of the bunching anchor 8 has become an unzipped portion 15 by pulling the outermost end, and FIG. 24-E shows complete removal.

The pulling can be started from any position on the implanted helical wire structure. FIG. 25 contains a sequence of four frames showing removal, after a four week implantation, from a live rodent by pulling on an approximate 5 cm helical wire structure of 100 strands, 25 micron diameter gold wire totaling 15 meters in length. The pulling is not from an end but from a middle portion 28. FIG. 25-A shows attaching with forceps 16, and FIG. 25-B shows initial pulling and unzipping. FIG. 25-C and FIG. 25-D show two unzipped portions 15 while the bunching anchor 8 remains unzipped, the latter showing the effectiveness of the anchoring in that unzipping has not yet reached the bunching anchor. This and other figures herein show that the unzipping can progress in two directions simultaneously and provides the clinician with the flexibility to choose the best access to the helical wire structure electrode prior to holding on to it with e.g., forceps to pull them out via multi-directional unzipping. When unzipping is done from a middle portion of the chronically implanted helical wire structure, then both directions unzip in parallel at relatively similar speeds and pulling forces for removal from similar tissues.

FIG. 26 contains a sequence of four frames of the same procedure in FIG. 25 showing pulling the electrode not from the end. Note that progressively starting in FIG. 26-B two sides of the electrode are unzipped portions 15 as they were pulled outside the rodent's skin in FIG. 26-C and FIG. 26-D.

The following photographs show clearly how the unzipping by pulling of the helical wire structure reduces the outer diameter and lengthens it. FIG. 27 is an image of a linear portion 9 of a helical wire structure, comprising a wire rope of 100 strands of 25 micron diameter gold wire, being pulled from the right side. Distance between coils 6 increases while outer diameter decreases, from right to left. The largest outer diameter on the left is 0.82 mm and on the right is 0.46 mm. The coil to coil distance on the right is 0.51 mm, and on the right is 1.53 mm. On the right, then, this is an unzipped portion 15. FIG. 28-A, FIG. 28-B and FIG. 28-C are three images of one embodiment of the same electrode herein pulled on the benchtop: upper is tightest and shortest, middle is part helical 1 and unzipped 15, and the lower is an unzipped portion 15, and is therefore the longest. FIG. 29 is an image (50×) of the helical wire structure having been pulled as in a removal. FIG. 30 is an image (100×) of a substantially linear portion of an electrode, comprising 100 strands of 25 micron diameter gold wire with an outer diameter of 0.75 mm, having been pulled and removed from a laboratory animal after implantation on a brachial plexus for 60 days. Tissue (shown as grey masses) has in-grown into the electrode. All the wire strands may have one or a few layers of encapsulation still attached to them after the helical wire structure has unzipped from the outer encapsulation tissue that remained inside the animal. It is also noteworthy that the single wire strands were able to slide against each other, further creating large local shear forces that aided with the unzipping of inner from outer encapsulation layers, thereby essentially applying a lever function between cell layers of encapsulation during the removal process. The unzipping is thus comparable to a reduction of removal forces on the macro scale similar to the use of a lever or an inclined plane to multiply forces by exchanging them for distance traveled, here the distance being the path the loops travel relative to each other and the outer encapsulation layers during the plastic deformation process that is part of the removal process.

FIG. 31 contains six images of a sequence of unzipping an electrode in ballistics gel 13. In FIG. 31-A, the bunching anchor 8 integral to the substantially linear portion 9 are fully encased by the gel 13, and the only void 12 is above the linear portion, where the dispenser leaving an insertion channel 14. In FIG. 31-B, removal pulling the linear portion has commenced, and the unzipped portion 15 fills the insertion channel, and a small void has opened in the upper left of the bunching anchor. In FIG. 31-C, at least half of the bunching anchor has been pulled upwardly into the insertion channel, and in FIG. 31-D only about a quarter remains. In FIG. 31-E the very last portion of the bunching anchor remains near the entrance to the insertion channel. In FIG. 31-F, the bunching anchor has been pulled totally out of the void, and only a portion remains in the insertion channel, above the drawn white line. In FIGS. 31-B to 31-F, the upper part of the insertion channel has been enlarged by side-to-side movements during the pulling process. The pudding-like material of the ballistics gel retained the shape of the bunching anchor that the helical wire structure had created. If the loops of the helical wire structure had moved longitudinally (laterally) to the ballistics gel then the coils would have sheared off the shape of the bunching anchor micro structure as the pudding-like material is not able to withstand large shearing forces. Because the helical wire structure electrode unzips one coil at a time by first contracting the coil inward towards the helical wire structure's center line before moving laterally to the walls of the cavity formed in the pudding-like ballistics gel, the forces pulling the coils first pull the coils away at a 90 degree angle from the ballistics gel before shifting laterally, thereby leaving the shape of the cavity intact as a “negative image” similar to how a cast retains the image of an object that it was formed around. The cast here of pudding-like material, shows that unzipping first moves inward (and away from the pudding) before moving laterally.

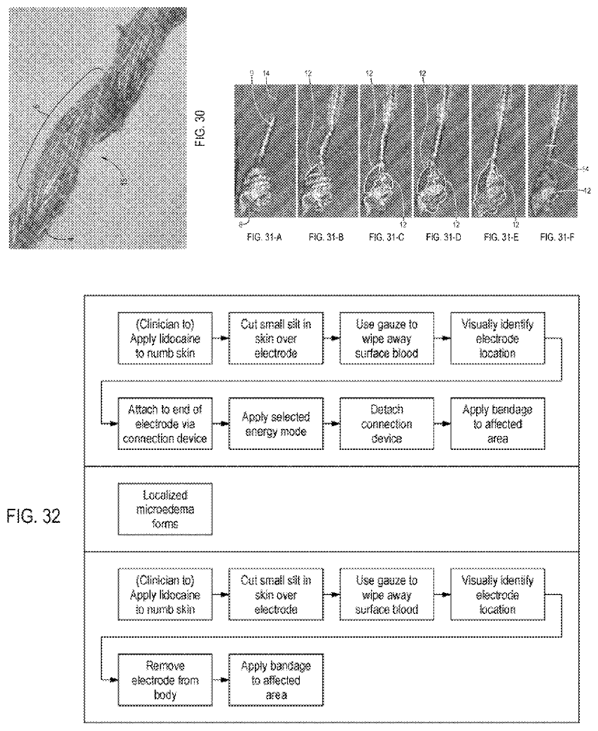

FIG. 32 is a flow chart showing steps for removal in a clinical setting.

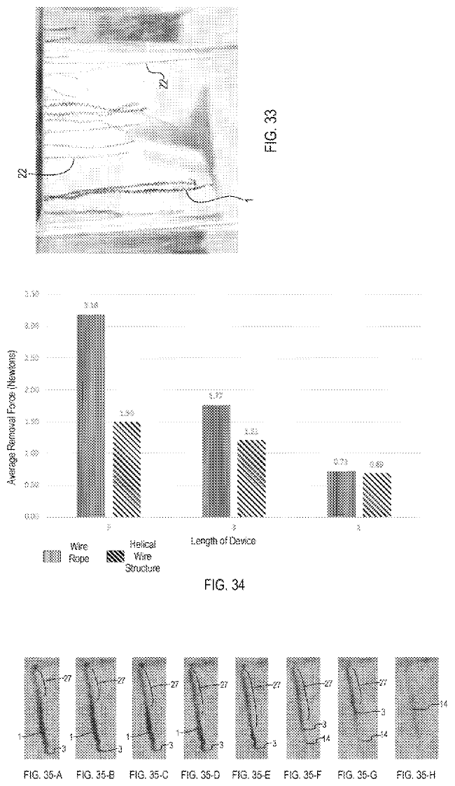

A comparison of forces for removal of the helix versus a wire rope of the same length shows the advance over the prior art made by the helix. The helix is strong enough to hold the structure, but not as strong as the folded, rolled, twisted, braided or mesh structure as in PCT/US20/061374, such as a folded mesh structure compacted to a wire to volume ratio of roughly 0.25 or less. FIG. 33 is an experimental set up of wire ropes 22 and helical wire structures 1 of equal length injected into ballistics gel 13. FIG. 34 is a chart depicting data comparing minimum force required to pull the wire ropes and helical wire structures from the setup in FIG. 33. The helical wire structure 1 require less force to remove because, due to the coiling, the helix is pulled out of the gel (or tissue) by a single coil at time, and the adherence to the surrounding tissue of only a single coil is being severed, whereas pulling the wire rope requires the physician to overcome the resistance of the entire wire rope because of tissue in-growth down the entire length of the rope. As shown in FIG. 34 the longer the helical wire structure, the greater the benefit from unzipping coil-by-coil compared to a wire rope of the same length. Moreover, the helical wire structure means that some of the coiled structure is not exposed to surrounding tissue, that is, it contacts itself and these areas receive insignificant in-growth. This highly significant in-growth in prior inventions is shown in images in PCT/US20/061374. Some of the structures there allow too much in-growth and some not enough.

Super-twisting or untwisting before and during removal may be achieved using an accessory attached to a low speed low torque power tool. Such an accessory will have a quick attachment feature to the power tool on one end and a malleable end with a split feature that can capture the helical wire structure electrode and it can be squeezed around and capture the helical wire structure electrode using fingers or forceps. Super-twisting the electrode in the direction of its initial twist before or during the pulling out adds torque to removal efforts. Super-twisting shears the electrode from in-grown tissue and makes removal easier because movement goes with strength of material instead of against. This super-twisting can be performed by hand or by machine, the latter being programmed and automatic. In one embodiment of the removal method, machine super-twisting is performed through torque limiting-super-twisting until a limit is reached or until a force threshold is crossed. A clinician can position removal device, clamp onto end of helical wire structure electrode, press a button, and be done. In another embodiment of the method, super-twisting or untwisting (against the initial direction of twist) and ablation are performed simultaneously or in sequence.

FIG. 35 contains a sequence of eight frames showing removal by super-twisting with an automated tool. In FIG. 35-C the super-twisted portion 27 is at the top and in succeeding frames super-twisting progresses until FIG. 35-G. Note how the end of the helical wire structure is essentially unmoved until FIG. 35-E. In FIG. 35-F, the end of the electrode has moved up the insertion channel leaving the insertion channel empty at bottom. In FIG. 35-H, all of the electrode has been removed.

Twisting in the same direction as the helix is super-twisting: it tightens the twisting in the same direction, taking advantage of relatively loose construction (a bundling of wires which can move independently of all others unless fixed by a mechanical stabilizer or insulator) and the hollow core which can be reduced or eliminated by the super-twisting. The effect is to compact the helical structure and reduce its outer diameter 17 and sever it from the in-grown tissue so the clinician may pull the electrode upwardly toward the skin through its former insertion channel or through the channel newly created by forceps or similar device. Mechanical stabilizers or insulators can prevent or lessen the amount of compaction and reduction of the outer diameter. Super-twisting aids with providing pulling forces due to the nature of tightening of the coils near the clamp holding the helical wire structure electrode.

The diameter of the helical wire structure contracts during the super-twisting, which is similar to some degree to what happens during unzipping by pulling alone described herein, but it is different from unzipping. That is, super-twisting compacts the helical structure so that it has a smaller outer diameter, and it also breaks the tissue connections to the electrode. The super-twist is in the same direction as the helical turns are, thereby tightening the helical structure of the helical wire structure. This tightening aids with the dislodging of the wires from the encapsulating surrounding tissue, mechanically separating the formerly stabilized wires of the helical wire structure. The super-tightening of the helical structure starts at a location close to the skin and slowly moves inward as more and more length of the helical wire structure dislodges from the surrounding tissue/material. The super-twisting of the helical wire structure results in a successive dislodging of the coils. This ability of the structure to dislodge successively allows for the separation forces to be acting in a very small volume and not on the entire length of the helical wire structure, concentrating the effect and facilitating the minimally invasive removal.

The minimally invasive removal process may also include alternating super-twisting and untwisting, pulling and pushing of the helical wire structure, to allow the outer diameter of the helical wire structure electrode to contract along its longitudinal axis, thereby aiding with the dislodging from the surrounding tissue. These may be applied in a wave-like or pulsatile form at, for example, an oscillation frequency of 2, 5, 20 or 100 Hz or frequencies in-between and up to the low kHz range to further reduce removal forces and increase removal reliability. Oscillating forces may be used to create a standing mechanical wave on the unzipped portion of the helical wire structure electrode to further concentrate unzipping forces on the coil that is still attached to the encapsulation tissue, using the standing wave to deposit oscillatory forces into the coil and aiding with separating the inner from the outer encapsulation tissue layers. This controlled release of the helical wire structure 1 is a designed feature allowing the minimally invasive removal of said structure along its encapsulation formed inside the body with a minimal risk for damaging tissues surrounding the channel housing the structure acutely or chronically. The mechanical (twisting and/or pulling) step may be preceded by a step of introducing energy into the body's cells surrounding the electrode 1 to disconnect the helical wire structure electrode physically, physiologically or chemically disconnecting from the cells forming the encapsulation and permeating the electrode to further lessen the mechanical holding forces prior to mechanical removal. There may further be a step of predetermined waiting time included between the energy deposition into the encapsulating tissue and the mechanical removal process, allowing the body to react to the introduced energy by creating a localized micro edema within the encapsulation and surrounding the helical wire structure electrode, further softening the cells in close proximity to the helical wire structure electrode, further reducing forces needed during removal.

Removal is assisted by applying a minimal energy through the helical wire structure itself (contacted by an energy-conducting probe) to produce the micro edema, and waiting for a pre-determined time to allow for a local temporary inflammation reaction to initiate, optionally with a non-invasive way of verifying that local temporary inflammation reaction has initiated successfully. The pre-determined time to wait after localized heating may be within a range one day to 21 days with a preference for approximate time point at around 7 to 10 days. During this wait time, the volume of the helical wire structure with surrounding tissues undergoes a local inflammatory response with fluid in-flow, forming a preferential channel for the electrode to be sitting in a local and temporary formation of a small micro-edema. This process is similar to how a splinter is removed more easily once a temporary inflammatory response has formed around the splinter and enables more easy movement inside the capsule formed by the inflammatory response, the electrode is more readily able to dislodge from the encapsulation tissue that had formed during the initial encapsulation that had happened in the first one to two months of tissue formation post electrode placement. Additionally, with all blood vessels within the cauterized, the tissues within the volume of the electrode are undergoing a local temporary inflammatory response as well, aiding with the local swelling and reduction of mechanical integration of the electrode with respect to the surrounding tissues. Prior to the removal via pulling on and unzipping the electrode, the clinician may choose to once more apply an application of heating to coagulate any newly formed vessels to further aid with an easy removal procedure and reduction of fluid egress from the wound after the electrode has been unzipped and extracted.

Removal requires a small cut (e.g. 3-5 mm) of the skin just above the implanted electrode to get physical access to the structure with an energy interface (i.e. ultrasound ablation or coagulation/cut or RF ablation probe/etc.) in order to not apply the heating right to the outer layer of the skin and aid with an improved healing experience for the patient by providing clean cut edges instead of ablated skin edges that would heal not as well as the clean cut ones. In some embodiments, the energy levels may be in the range of 5 to 25 W and application duration may be a few seconds for Ultrasound or RF; In some instances, the energy levels may be in the range of 26 to 100 W and application duration may be very few seconds (<5 s) for any kind of energy; In some cases, the energy application may be repeated a few times with ultrasound or impedance measurements (vs. a distant return) being used either right after the ablation energy application or at the expected removal time point to assess sufficient ablation volume and/or micro-edema formation. In some cases, the energy transfer may be achieved by first pushing a thin needle or wire right along the helical wire structure electrode, contacting it in multiple points, prior to applying the energy (electrical, ultrasound, heat/cool) to the electrode.

### ADDITIONAL ASPECTS

In other embodiments, the helical wire structure may be the front end of a system that incorporates the front end for bunching and followed by an insulated lead portion that does not have the ability to fold when placed into tissue, potentially followed by a connector or an interfacing unit to be able to connect to another foldable structure, interconnecting structure or implantable pulse form generator. Such a system uses the helical wire structure to provide mechanical anchoring forces that are established during the placement procedure and allow for the entire system to be left inside the body without the need to be mechanically anchored further with the aid of sutures or other traditional techniques.

There may be locations of higher stiffness (e.g., via added glue, heightened tightening via rolling or other means of mechanical stiffening) of the manufactured wire structure along its longitudinal axis and there may be locations of lower stiffness (e.g., by lesser rolling or lesser compression during the out-of-body manufacturing process) to allow for predetermined locations of folding during the deployment process. An example is for the helical wire structure to be stiff for 2 mm in length followed by 1 mm in lesser mechanical stiffness. Another example is for the manufactured helical wire structure to be stiff for more than 2 mm in length (such as 5 mm or 10 mm or 20 mm) followed by 0.5 mm (or more or less than 1 mm) in lesser mechanical stiffness. Such a helical wire structure can be shown in the following example such as a 2 mm length which is stiffer and 1 mm length which is less stiff along its longitudinal axis is be able to create a helical wire structure electrode of meandering waves of approximately 1 mm amplitude waves height (half the 2 mm stiff length) during the placement process.

The helical wire structure allows for a liquid, gel or gas (or even mixtures), to pass around the helical wire structure or through the hollow core of the helical wire structure. This passage way allows for the injection or removal of said liquid, gel or gas into the body to aid with the formation or modification of the void volume prior to the injection of the helical wire structure or during the injection of the helical wire structure.

During injection, the helical wire structure may be pushed into and retracted (fully or partially) into the cavity to enable creation and filling of the cavity within or adjacent to the target tissue.

Suture glue, bone cement or other in-body curing polymers or amalgams may be placed prior to, during, or in parallel to injection or after a specific portion of the helical wire structure has been placed to aid with anchoring or to enhance long term mechanical stability. Said polymer may be injected through the same needle lumen as the helical wire structure or it may be injected by a secondary dispenser channel either integrated into or separate from a first dispenser channel.

The helical wire structure may be manufactured outside the body from wire made of gold and other metals listed elsewhere herein, polymers such as Prolene® suture (11-0, 10-0, 9-0 down to large diameters as 1-0) or combinations in order to provide biocompatibility and mechanical properties for bunching, retention, integration, and later minimally invasive removal capabilities.

After the electrode has been placed, the wound (e.g., needle puncture) may be closed with sutures, steri-strips or suture glue.

Following integration by the body's encapsulation reaction to the electrode, the helical wire structure may be used to transfer energy to or from the target tissue inside the body to another location within the body, such as a location closer to the skin (i.e., a subcutaneous pocket at a depth of about 1 to 3 mm inside the body).

The electrode may be used to transfer electrical, thermal, acoustic, mechanical or electrical energy to initiate a dislodging process from the tissue surrounding the electrode.

In one embodiment, the helical wire structure may be engaged with a small needle from the outside of the body, the shaft of the needle being insulated, the tip of the needle intended to mechanically and electrically engage inside the body with the electrode not being insulated. This thin needle (i.e., 25, 27, or 30 gauge needle or smaller or larger) allows for the repeated transfer of electrical energy into the helical wire structure at any time after injection and even long after a stable encapsulation has formed inside and outside of the helical wire structure electrode. Because the electrode is being permeated by the body's own cells and vessels the engagement via needle from the outside of the body may be a repeated procedure with the intent to enable a direct metal to metal connection to the world outside of the body at very low (<100 ohm, in certain cases<10 ohm) impedances.

In certain instances, the helical wire structure may be used to supply electrical stimulation or blocking energy to neural tissue, or longer-term electrical blocking energy that induces the changes of the pH in the direct vicinity of the electrode, such as placed in close proximity to neural tissue to allow for an electrically induced chemical lesion (injury/dissolution) of a tissue target, or electrical current to induce a change in pH in the direct vicinity of some or all of the wires of the helical wire structure electrode to induce cell death of the first few cell layers mechanically anchoring the electrode. This chemical lesioning may be used minutes, hours or days preceding a minimally invasive removal procedure. The electrode herein is highly conductive for electric current but also other forms of energy including radiofrequency current, microwave current and direct current, as well as ultrasound.

The electrode may be optimized for a minimally invasive removal days, weeks or months after the placement procedure. Such a removal may be achieved by pulling, twisting, turning or otherwise mechanically engaging with the electrode.

### Integrated Conventional Lead

The present invention may be used as a standalone device or may be connected to a conventional lead which, in the prior art, is a single or group of wires insulated via a weld and or adhesive, or the connection may be established mechanically and stabilized via an adhesive. The adhesive may be non-conductive to provide electrical shielding and limit galvanic interactions between the wire structure and lead wire materials, or it may be conductive to ensure a more robust electrical connection. A conventional lead may have a helical wire structure connected on each end, with needle injection devices of different lengths. One side may be injected to a target, with the other injected subcutaneously.

During the stranding and winding process, or thereafter, a conventional lead 24 may be indirectly integrated to the helical wire structure or partial coating applied to the wire structure to form lead segments for a directly integrated lead. In the mono-polar setup, this lead integrated wire structure connects an anatomical target to some distal point. In the bi- and multi-polar setup, this lead integrated wire structure connects multiple target points to multiple distal points in a device.

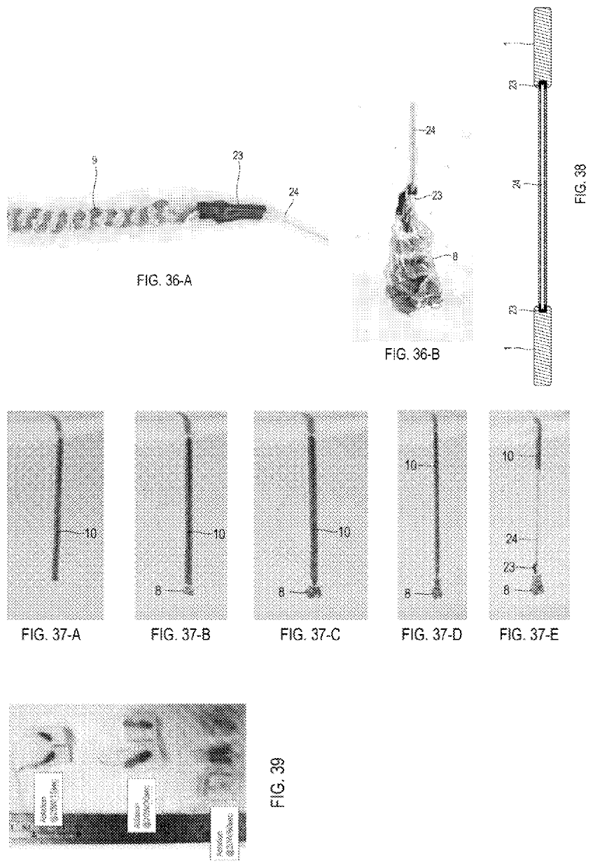

FIG. 36-A shows a portion of an electrode comprising a helical wire structure's substantially linear portion 9 and stainless steel wire lead 24 connected and surrounded by heat shrink polymer. FIG. 36-B shows, after injection into ballistics gel, the bunching anchor 8 connected to the stainless steel lead 24.

FIGS. 37-A to 37-E show a sequence of injection of the type of electrode in FIGS. 36-A and 36-B. In FIG. 37-B and FIG. 37-C, the bunching anchor 8 forms, and in FIG. 37-D the stainless steel lead 24 emerges from the dispenser tip as the tip is withdrawn and this lead is connected to the bunched portion. In FIG. 37-E, the stainless steel lead is shown further injected and connected to the bunching anchor 8 at the connector 23.

FIG. 38 is a schematic diagram showing a stainless steel lead 24 in the center connected on each end to a helical wire structure 1.

### Energy-Assisted Removal

When an electrode moves inside its own encapsulation, this is sometimes called “pistoning of the lead.” A method of the present invention is to generate pistoning directly prior to removal. Applying energy through the helical wire structure either prior to or during removal creates a margin around the electrode for pistoning and subsequently removal to occur as a unit. Energy is applied that causes small local cell die-off and influx of bodily fluids, allowing for pistoning. In one embodiment of the removal, several days later the entire helical wire structure electrode is removed by a slight incision in the skin and the clinician, holding on with forceps, slowly pulls it out. This minimal margin of cell death around helical wire structure electrode is a minimum of 10 microns (about 1-2 cell layers) and a maximum of 1 mm. After application of the energy, waiting several days allows for localized micro edema to form as the body's natural response. At the time of removal, this results in a fluid-filled pocket containing a single unit of helical wire structure electrode material. A small slit is sufficient to pull the helical wire structure electrode and take out without open cut down. The amount of bleeding from this removal method is smaller than that for traditional removal of electrodes and leads. It is a straightforward minimally invasive procedure which greatly minimizes tissue damage and recover from a removal of a neuromodulation device. The steps for the clinician include:
Option 1 Applies lidocaine, Marcaine (bupivacaine hydrochloride) or other analgesic or anesthetic and then cuts down, uses gauze to wipe away surface blood Has visible access to helical wire structure electrode, clamps on with connection device (alligator clip) to end of helical wire structure electrode (tail, or tail formed into a second skeined bundle for collector). Applies energy mode Waits several days for localized micro edema to form Removes helical wire structure electrode from pocket
Option 2] Does not need to apply lidocaine, Marcaine (bupivacaine hydrochloride) or other analgesic or anesthetic. Does not need to gain open access. Uses a needle based approach to conduct direct current or high energy alternating current into the helical wire electrode structure as energy mode. Or a combination of DC first and then radiofrequency (or coagulation/cut) currents.
Option 3 Does not need to apply lidocaine, Marcaine (bupivacaine hydrochloride) or other analgesic or anesthetic. Does not need to gain open access. Uses ultrasound based approach to conduct alternating mechanical energy into the tissues in direct vicinity of the helical wire electrode structure as energy mode.

In addition to standard electrical stimulation applied to implanted electrodes, the helical wire electrode may be powered by a radiofrequency ablation generator. The application of RF energy through monopolar or bipolar configurations results in a coagulative necrosis of tissue directly surrounding an electrode. With heat-induced denaturation and randomized restructuring of native proteins, there occurs a shear modulus difference between non-ablated and ablated tissue. By manipulating orientation, and applying low powers over pulsed periods of time, creation of ablation zones with selectively small, sub millimeter margins is possible. RF energy may be applied through clamps or needles. Minimizing unnecessary tissue coagulation is key for this method of removal. FIG. 39 is an image of gel with helical wire structure electrodes, having been heated with RF energy each at 20 watts, in 15, 30 and 60 second intervals. The amount of tissue heated per interval is shown I Table 1:

TABLE 1

| Seconds of 20 W heating | Volume in cm³ |
|---|---|
| 15 | 0.2136283 |
| 30 | 0.788932455 |
| 60 | 1.145110522 |

The normal potential gradients that exist across cell membranes are disrupted by electric pulses that generate surrounding field intensities of 0.5 to 1.0 kV/cm. A sufficient alteration of cell transmembrane potential creates disruptions of cell bilayers and permanent pores that irreversibly increase surrounding cell membrane permeability in a phenomenon known as electroporation. Irreversible electroporation (IRE) via applied pulsed, low-energy direct current between a helical wire structure electrode and an inserted probe, or between helical wire structure electrodes in bipolar configuration, results in focused apoptotic cell death surrounding or directly contacting the implant. This cell damage triggers fluid influx into a precise zone around the implant, a tolerable and mechanically advantageous environment for helical wire structure electrode removal through pistoning. Because IRE is non-thermal and solely affects the cell membrane, IRE is unaffected by heat sinks caused by vascular flow, and spares extracellular scaffolds which preserve structures such as nearby blood vessels and nerves.

Tissue coagulation through the helical wire structure electrode as an active electrode may be achieved through the application of high voltages, typically between 3000 and 9000 volts, using intermittent frequency to generate heat and consequently evaporate intra-cellular water while coalescing cytoplasm. The larger volume of the helical wire structure electrode helps mediate applied power density, avoiding explosive vaporization of interstitial fluid. Careful cauterization of tissue immediately surrounding the helical wire structure electrode may similarly facilitate its removal. Standard coagulation/cut units as available in most operating rooms may be used to supply sufficient energy for less than 10 seconds to cause induced controlled cell death of the first few (inner) layers of the encapsulation.

Heat can be applied to the helical wire structure electrode to diffuse to surrounding tissue. Cold can be applied to freeze tissue. Thermal energy propagates slowly down length as opposed to diameter and manifests more strongly at insertion point.

In ultrasound ablation, high-frequency vibration is used to cut or cauterize tissue. Surgical units typically contain clamping and cutting tips. A clamping tip may be used to attach to the end of the helical wire structure electrode before delivering energy to tissue.

Lithotripsy involves a focused ultrasound beam and utilizes the modulus difference between different tissues, and literally shakes apart kidney stones into particles small enough to be removed. Lithotripsy has an interesting intersection with helical wire structure electrode/tissue modulus difference. Even when encapsulated, the helical wire structure electrode maintains the elastic modulus of the metal it is made of and tissue remains tissue. Using a focused beam of vibrations can separate those two based on their physical properties, thus working with multiple shapes and orientations

Removal as described herein is advantageous because it uses unique the geometry of helical wire structure electrode to combine loosening movement with removal motion. It is less likely to disrupt interior body structures because of the small lateral/rotational axis of movement as opposed to large proximal axis of movement. It is able to remove through the path of insertion or the electrode's own footprint. Ablation makes removal straightforward and bloodless by cauterizing only the blood vessels contacting helical wire structure electrode.

The following combination approaches for removal apply: Application of ultrasound energy followed by twisting allows removal in one step. Application of ultrasound energy application followed by waiting for a range of one hour to week, say three days, and then by twisting to remove. Electrical energy application at high frequency (100 kHz up to 1 GHz) to cause heating of the tissue in direct contact with the electrode and beginning protein destruction or even coagulation, then followed by twisting to remove right away. Electrical energy application at high frequency (100 kHz up to 1 GHz) to cause heating of the tissue in direct contact with the wire structure and beginning protein destruction or even coagulation, then followed by waiting for a range of one hour to one week, say three days, and then by twisting to remove.

### Mono- and Multi-Polar Lead Integrated

In other embodiments the helical wire structure electrode also comprises an integrated conventional lead. The lead may be directly or indirectly integrated, as achieved through a combination of fabrication methods. Integration of the lead alters the cellular ingrowth and mechanical integration within desired regions, while providing electrical connections between wire structures located at different anatomical targets. The lead integrated wire structure has mono-polar and multi-polar design configurations, allowing for interfacing with successive anatomic targets within one injection window and procedure. Delivery of the lead integrated wire structure is achieved either through standard dispenser channel methods commonly used to place leads, or may be achieved through front-loading of the interfacing bare wire structure regions only that do not comprise the lead region, which is placed in parallel to the wire structure delivery cannula. The latter approach simplifies the delivery mechanism to accommodate an overall shorter, and therefore more pushable, wire structure. This wire structure may also be delivered at incident angles and in complex curvatures using curved or bendable delivery needle systems.

As described herein, the helical wire structure comprises multiple stranded wires, are made of an inert conductor such as platinum or gold, and may be partially coated with inert materials such as Parylene C, Fluorinated polymers, or Silicones to form “lead” regions. Coating schemas of the wires will modulate cellular ingress rate and ability, with effects on mechanical properties of the device and required removal forces. Coating may be achieved either by micro coating of individual wires versus macro coating of multiple wires. Coating of individual wires with <15 micron (especially<5 micron) coatings will maintain mechanical flexibility and biological integration of the mesh structure to mechanically anchor the device. Coating of multiple wires, such as conventional leads, are not readily integrated and provide a smooth/slippery biological conduit that allows for free movement of the lead. Inert coatings, dependent on thickness, also provides electrical or thermal insulation from surrounding tissue at both the micro and macro scale

FIG. 40 is a schematic drawing of a helical wire structure electrode with bunching anchors connected by intermittent portions of macro-coated helical wire structure shown with an insulator 19 and portions with individually micro-coated wires 26. As shown in the insets, the wires are uncoated helical wire structure in (a), individual wire strands which are micro-coated in (b), and the entire helical wire structure macro-coated in (c). These differing segments are embedded in several tissue layers: the bunching anchors 8 underneath the skin and on a tissue target to enable energy transmission to or through the tissue; the macro-coated helical wire structure in the muscle planes; and the micro-coated strands in fascia/adipose to enable relatively more in-growth for insulated wire than allowed by the macro-coated. The greater in-growth among the micro-coated wires creates a tighter connection than the macro-coated sections.

There can be indirect integration of a lead wire, optionally that may be coated with another material or mechanically achieved with the wire structure through interweaving and twisting of the lead materials' conductive wire element and the wires of the wire structure. The indirect integration step may occur during or at the end of the fabrication of the wire structure. A mechanical connection alone is sufficient. Indirect integration of a lead wire to a wire structure is also achieved through the use of welding or brazing approaches. Following welding, brazing or crimping, the connected surface may be treated and coated as necessary

There can also be direct lead integration into a wire structure with partial coating of the wire structure at intermediate stages of the fabrication process. Coatings may be achieved through dip-spin coating of polymers or via vapor deposition methods. Coatings must be flexible to withstand intended torsion and folding of the wire structure during placement and use. The surface of the wire structure may be treated to improve adherence and allow for improved structural integrity of applied partial coating.

FIG. 41 is schematic diagram showing the layout for an electrode comprised entirely of a mono-polar helical wire structure, with a central region insulated 19 and the two ends uninsulated 1.

The mono-polar lead 24 integrated with a helical wire structure connects a helical wire structure placed at the anatomical target of interest to a distal energy generator or input site. The distal portion of the mono-polar lead integrated wire structure may be another wire structure or terminate in a lead for direct hookup to energy sources. The mono-polar lead integrated wire structure may be directly integrated into the lead for continued wire connection from the target of interest to the distal site, or indirectly via welding/brazing that provides a conventional lead connection between two wire structures or a wire structure and an electrical source via the aforementioned lead.

In other embodiments two or more wire structures with integrated leads may be combined into one wound helical wire structure that has multiple staggered bare wire structure regions that allows for successive electrical connections isolated from each other into one wire structure. The winding pattern may be regular with equally spaced regions between each wire structure in the multi-polar parallel pre-stranding to post winding product, or may be intermittently altered where one structure may traverse several other structures over the course of single winding.

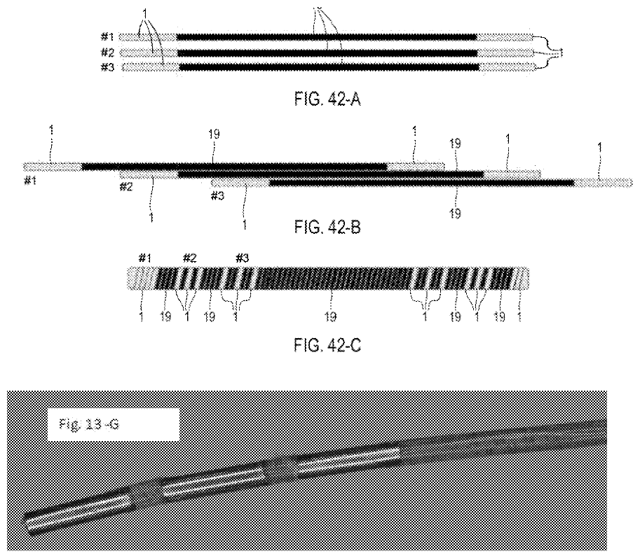

FIG. 42 shows a layout diagram for how to build one embodiment of a multi-polar electrode comprising a helical wire structure comprising 3 wire ropes (#1, #2, #3). In FIG. 42-A three wire ropes 22 are shown as numbered with two uninsulated ends and a central insulated region. FIG. 42-B shows the three wire ropes staggered in parallel to offset the insulated regions over the insulated regions. In FIG. 42-C, the three wire ropes have been wound together to form a multi-polar lead integrated wire structure. The uninsulated wire ropes are spaced throughout the length of the electrode, allowing control over energy transfer as well as in-growth of bodily tissue.

Aside from standard in-line delivery of an injectable mono-polar wire structure with integrated lead, ease of delivery may be achieved through front loading of only the bare interfacing region of the wire structure into a delivery system and leaving the lead region external to the wire structure delivery cannula. One or both ends of the wire structure with integrated lead in a mono-polar configuration may be delivered using such a front-loading and deliver approach.

The lead wire section may further be deployed with a multi-barrel needle: two or more needles mechanically attached where one needle holds the helical wire structure intended to form the anchor section and one needle holds the lead section. The figure above shows only the needle holding the helical wire structure intended to form the anchor section for a simplified device. The discussion elsewhere herein about injection of a deterministic volume of helical wire structure is also relevant here to the electrode comprising an integrated (connected) lead. A multi-barrel dispenser allows the limitation of how much helical wire structure can be injected maximally thereby limiting the diameter of the bunching anchor and thereby reducing risk of putting unwanted pressures or forces on a nerve or ganglion, especially if that nerve or ganglion is in a location of limited space such as foramen. This capability enables clinicians to reliably inject the right amount of helical structure for the bunching anchor without the risk of injecting too much, potentially creating an anchor too large in size and with the risk of pressuring the neural structure (nerve, ganglion, plexus) passing by.

TENS electrode patch electrodes may be placed on the skin adjacent to the uninsulated portions of lead part A and lead part B to allow for a multi-polar stimulation interface next to the nerve with a stimulation signal provided by an outside-the-body generated waveform. Interfacing may further be achieved with small needles penetrating the skin and providing a metal to metal connection instead of a purely capacitive coupling to the subcutaneously placed uninsulated portions of the injected helical wire structure. Waveform signals may include stimulation signals, short-term temporary nerve blocking waveform signals, combinations thereof, or short to long term nerve blocking waveforms such as radiofrequency signals or direct current.

Delivery of the wire structure, with or without integrated lead, may be achieved through curved needles or flexible cannulas. Potential targets that are amenable to delivery at incident angles or with complex curvature include but are not limited to the following: nerves, externally or into the sheath; anatomical targets located behind sensitive tissues (e.g., blood vessels, nerves, nerve plexus or ganglia) or bony structures; between muscle planes; and into or around tumors

FIG. 43 is an image (13.1×) of an electrode, comprising 100 strands of 25 micron diameter gold wire with an outer diameter of 0.75 mm, in a flexible cannula of 1 mm outer diameter. The path away from a target tissue back to the skin is following the curvature of the intruder when said introducer is being retracted after the formation of the anchor point. This way the flexible helical wire structure is leading away from the nerve at a shallow angle initially and reaches the skin at a steeper angle where it may be formed into yet another anchor point if so desired.

Embodiments include straight needles with slanted/notched tips, straight needles with side ports, straight needles with side ports and inside flexible tubes intended to exit at a 10 to 90 degree angle from the side port with the helical wire structure electrode being able to then exit from the bent tube, or curved needles with front or side ports.

In one embodiment delivery of the wire structure is accomplished with a multi-barrel needle. The advantage lies in the ability to deploy larger lengths of wire structure without friction inside of the needle limiting the total length of or deployable wire structure.

FIG. 44-A shows the tip of a dual, or double barrel, needle. In FIG. 44-B two portions of a single electrode have been loaded into each barrel from the tip, as shown by the portion of electrode visible between the two barrels. FIG. 44-C shows that a portion of the electrode has been push from each barrel. While double and triple barrel needles are the most applicable versions, there may be even more barrels combined to form a larger overall diameter deployment device. FIG. 45 contains a sequence of images of injection of an electrode comprising a wire rope of 100 strands of 25 micron gold wire having a 0.25 mm hollow core, outer diameter approximately 0.75 mm, from a double barrel needle into ballistics gel. The length of the electrode prior to injection was 8.5 cm, and the wire rope was made from 25 m of continuous wire. FIG. 45-A is with the electrode inside the needle, and FIG. 45-B shows a first bunching of the electrode from the barrel on the right after being pushed fully out of the right barrel by the plunger while substantially stationary, and then FIG. 45-C shows the portion in the left barrel has also exited that needle and deposited in a substantially linear way while the needle was withdrawn. In FIG. 45-D, the left barrel continues to deposit a linear portion, and in FIG. 45-E the needle has stopped while continuing to push the electrode so that it bunches in a second location. FIG. 45-F shows the first and second bunched areas integral with the substantially linear portion of the electrode.

Multiple barrel dispensers (such as in FIGS. 44 and 45) allow the clinician to deliver a deterministic volume of helical wire structure at a given location by injecting a pre-determined and pre-measured length of the helical wire structure from a particular barrel in a specific area. This has application, for example, in placing the electrode in a spinal foramen, say, a 3 cm length in a lumbar foramen for a DRG placement and a 5 cm length in a sacral foramen for an overactive bladder (OAB) placement.

The helical wire structure may be deployed with an additional lead attached to the structure when leaving the multi-barrel deployment device

Both ends of the gold wire structure with integrated lead may be frontloaded into needles with cannulas of similar or differing lengths to deliver the bare wire structure ends of the mono-polar structure to two different anatomical targets.

A dual needle is a preferred device for insertion of a helical wire structure with integrated lead, with at least one bunching anchor near either end are significantly larger in diameter than the diameter of the undeployed helical wire structure. The ability to deploy a multi-anchored structure, with a dual needle device, is a further tool allowing for sutureless anchoring inside the body.

The helical wire structure, in several embodiments, further comprises at least one immunoreactive agent selected from a group consisting of cells, whole blood, blood serum, biodegradable polymers sugars, amino acids, proteins, iron, lipopolysaccharides, collagen and hyaluronic acid. at least one anti-inflammatory agent selected from a group consisting of steroids, antioxidants, superoxide dismutase mimetics and non-steroidal anti-inflammatory drugs. at least one hemostatic agent selected from a group consisting of microfibrillar collagen hemostat, chitosan, kaolin, zeolite, anhydrous aluminum and sulfate. at least one folded wire structure in another embodiment is at least one pharmacological agent selected from a group consisting of an antibiotic, an anti-fungal, an analgesic and an anesthetic. At least one contrast agent for visualization during ultrasound, fluoroscopy or x-ray selected from a group consisting of Fluorescein and platinum.

### FURTHER ASPECTS OF THE INVENTION

The invention has the additional following aspects which may be included in or in addition to what is described above.

Disclosed herein is an injectable electrode comprising a helical wire structure configured for full and chronic implantation on or near a tissue target in a body for conduction of energy to or from said tissue target, said helical wire structure comprising at least one wire rope comprising a plurality of strands of biocompatible and conductive wire, said helical wire structure enclosing a hollow core within an inner diameter and having a longitudinal axis, an outer diameter and two ends, said helical wire structure having flexibility for self-bending in any direction up to 180 degrees on said longitudinal axis, and being configured for loading into a dispenser capable of creating an insertion channel to the tissue target, and at least a portion of said helical wire structure being capable of self-forming a bunching anchor wider than said insertion channel when injected while said dispenser is substantially stationary, so that said bunching anchor is capable of securing said electrode in place. The electrode is capable of forming a substantially linear portion when exiting from said dispenser when said dispenser is moving. As a result of the foregoing, the electrode is further configured for minimally invasive removal so that, when the helical wire structure is pulled from a middle portion or near one of said ends, the longitudinal axis lengthens by reducing said inner diameter and said outer diameter. In one embodiment, the hollow core is at least partially filled by a guide wire prior to and during injection, and the guide wire is configured to be withdrawn during or after implantation by a clinician. In another embodiment the electrode further comprises a conventional lead wire affixed by a connector to the helical wire structure by means selected from the group consisting of welding, looping, gluing and heat shrinking a polymer. The electrode in another embodiment has a portion of the strands with a different diameter or a different conductive material and these may be concentrated in different portions of the helical wire structure having different bending capabilities. The biocompatible and conductive wire or wires can be one or more of many different metals selected from the group consisting of gold, silver, platinum, stainless steel, titanium, titanium-nickel, iridium, platinum-iridium, tungsten, platinum-tungsten and other metal alloys including ultra-high-strength, nickel-cobalt-chromium-molybdenum alloy such as MP35N. The wire rope may also contain strands of nonconductive material in other embodiments, and/or have at least one mechanical stabilizer and/or insulator affixed to a portion of the helical wire structure. The hollow core is configured for passage of liquids, glues, gels or gasses during injection. In other embodiments, the bunching anchor is generally of an irregular shape. In other embodiments, the wire rope comprises a single strand of continuous length which has been folded and then twisted. The energy conducted is selected from the group consisting of alternating current, pulsatile current, direct current of alternating amplitude, direct current of constant amplitude, radio-frequency ablation current, microwave current, monopolar pulsatile and bipolar pulsatile current, or combinations thereof.

A method of manufacturing the injectable electrode with an outer diameter includes forming strands of biocompatible and conductive wire into a wire rope, and then twisting the wire rope into a helical wire structure around a guidewire. The method may also include application of a mechanical stabilizer or insulator to at least one portion of said helical wire structure. To achieve variations in properties such as bending characteristics, forming the strands into a wire rope comprises providing multiple strands with different diameters or different materials, and the forming further comprises wrapping the different diameters or different materials along different subsets of mandrels thereby creating different skeins, and then combining and twisting the different skeins, so that different portions of the helical wire structure possess concentrations of the different diameters or different materials. In different embodiments, the outer diameter remains consistent within a tolerance of plus or minus 25%, or plus or minus 10% or less than 5%.

The invention provides a method of injection of the helical wire structure electrode comprises at least one wire rope having a first direction of twist and loaded into dispenser, by guiding the dispenser to a tissue target, and then forming a bunching anchor on or near the tissue target by pushing said electrode from the dispenser while substantially stationary. In another embodiment, injection allows the forming of a substantially linear portion by pushing said helical wire structure from said dispenser while moving. Injection may also include twisting the dispenser in a second direction opposite the first direction of twist and thereby loosening the helical wire structure in specific locations to allow for greater tissue in-growth. During injection, a fluid, gel, gas or glue may travel through the hollow core and from the dispenser and, in one embodiment, the fluid comprises water and is pushed at sufficient volume and pressure to dissect tissue plains and/or create a void near the dispenser. In another embodiment, the glue comprises bone cement or tissue glue and the dispenser is near a bony cavity.

A method of anchoring the helical wire structure electrode herein includes providing a dispenser loaded with the electrode, then inserting the dispenser to a tissue target having a sheath and thereby creating an insertion channel, and then pushing the helical wire structure from the dispenser while substantially stationary such that at least a first portion of the helical wire structure forms at least one bunching anchor on or near the tissue target and has a width greater than the insertion channel. In one embodiment, the method of anchoring also includes moving said dispenser and depositing a second portion of the helical wire structure which is substantially linear in or near the insertion channel. In yet another way of anchoring, the at least one bunching anchor is located at least partially within the sheath of the tissue target.

A minimally invasive method of removing the electrode herein described in these further aspects includes pulling the helical wire structure from a middle portion or one of the ends so that the helical structure lengthens by reducing the inner diameter and the outer diameter, and thereby loosening the electrode from tissue in-growth. In another embodiment, removal includes the step of pulling and then a step of applying energy selected from the group consisting of DC, RF, MW, HIFU to the electrode to coagulate nearby vessels, char tissue in-growth and/or create a micro-edema. In one embodiment the application of energy comprises providing heating the electrode to approximately 50 to 60 degrees C. In another embodiment the method of removal includes, before pulling, first injecting saline solution around the electrode before pulling the electrode from one of the ends. In another embodiment the method includes super-twisting the helical structure from the middle portion or one of the ends while pulling.

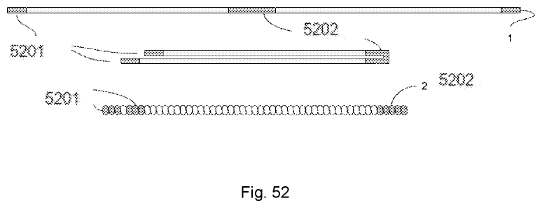

Referring to FIG. 52, loopback electrode configurations are illustrated showing manufacturing sequences for helical wire structure electrodes with loopback designs. A loopback electrode configuration produces a helical wire structure electrode with two exposed electrodes at a proximal end placed on an electrical stimulation target and one exposed electrode at a distal location near or under the skin. The helical wire structure is fabricated from a single wire rope structure with exposed ends that form proximal electrodes and an exposed region offset from a center of the wire rope structure that forms a distal electrode. The wire rope structure is folded back at the distal electrode near the middle of the rope to produce two parallel ropes with the proximal electrodes offset from each other so as to not directly overlap. The resulting U-shaped parallel rope wire structure is wound on a core to produce a helical wire structure electrode with a loopback design. The loopback electrode configuration is reversible so that the loopback central electrode is placed on the stimulation target proximally, producing a helical wire loopback structure electrode with one proximal electrode and two distal electrodes instead of two proximal electrodes and one distal electrode. Two or more helical wire loopback structures are combined by offsetting the loopback rope wire structures at a predetermined distance, placing them in parallel, and then winding the parallel structure around a mandrel to create multiple loopback electrodes.

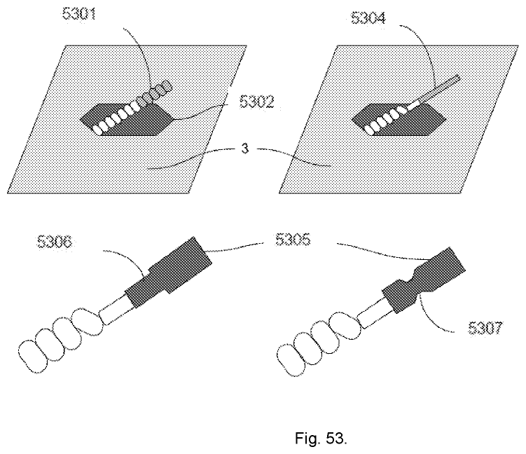

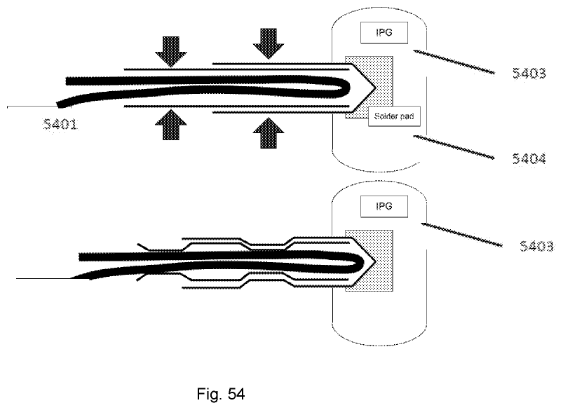

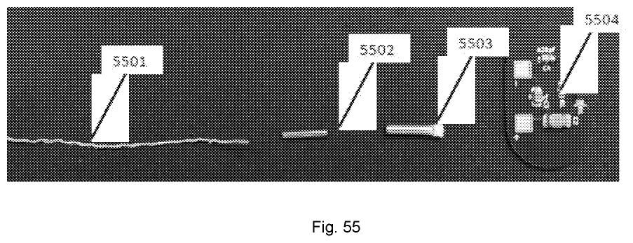

Referring to FIG. 53, FIG. 54, FIG. 55, and FIG. 53, connector systems for adding connections to previously implanted electrodes are illustrated. A previously placed helical wire structure electrode is connected to an implantable pulse generator by applying a cylindrical conductive connector to the implanted helical wire structure electrode without complete removal of the electrode from the body, with the intention to maintain the proximal end of the electrode unmoved. The implanted helical wire structure electrode has the distal component under the skin partially extracted to extend the structure back to its non-helical rope wire structure configuration. The partially elongated electrode end in a rope wire structure configuration is cut to length, after which a cylindrical contact is applied by threading the wire rope structure into the cylinder. The cylindrical conductive contact is secured to the electrode using either a crimping force or distance, or alternatively using a predetermined swaging diameter reduction to mechanically and electrically secure the cylinder to the electrode. The permanent electrode electrical and mechanical connection is made using a ferrule or segment of tubing less than 5 mm in length with an inner diameter that is greater than the diameter of the exposed electrode contact. The ferrule or tubing is manufactured from materials that do not cause galvanic corrosion with the contact material of the electrode. An insulative sleeve is either slid over the electro-mechanical connection point, or a heat shrink is applied, or an insulative adhesive is applied to limit current leakage from the connection point. The connection on the implantable pulse generator is direct through the use of a secondary ferrule or tube that has an inner diameter greater than that of the primary ferrule or tube, which is directly welded onto the implantable pulse generator.

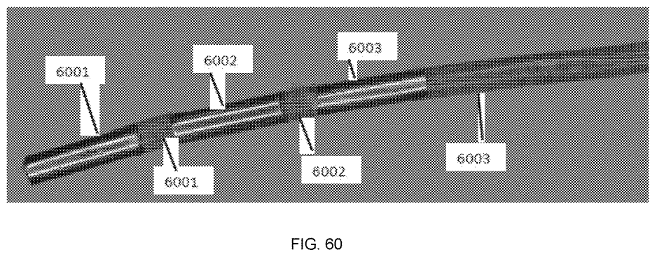

Referring to FIG. 60, a multi-contact connector assembly is illustrated showing multiple cylindrical connectors on a distal end of a multi-contact helical wire structure electrode. The cylindrical conductive contacts are separated by an insulative material from each other. Individual conductors of the multi-contact helical wire structure electrode are connected to each individual cylindrical contact through welding, brazing, or using a conductive adhesive. The insulative material separating cylindrical conductive contacts partially extends into the remainder of the helical wire structure. The multi-connector configuration is used for electrical stimulation or other applications such as radiofrequency ablation.

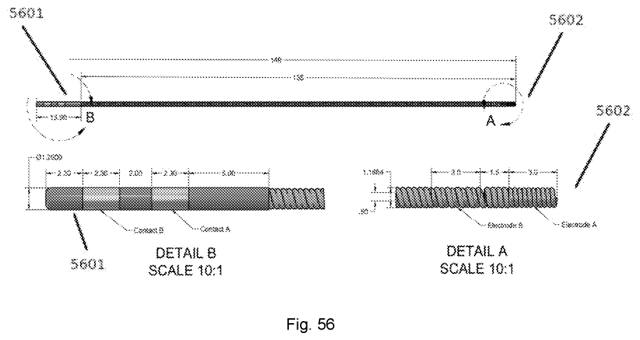

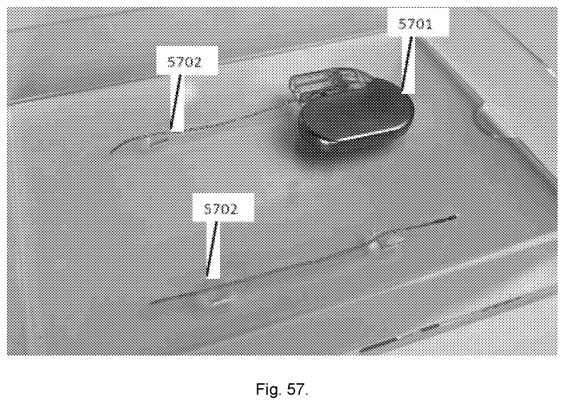

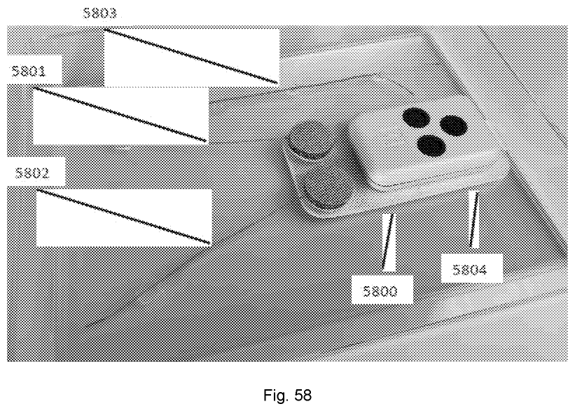

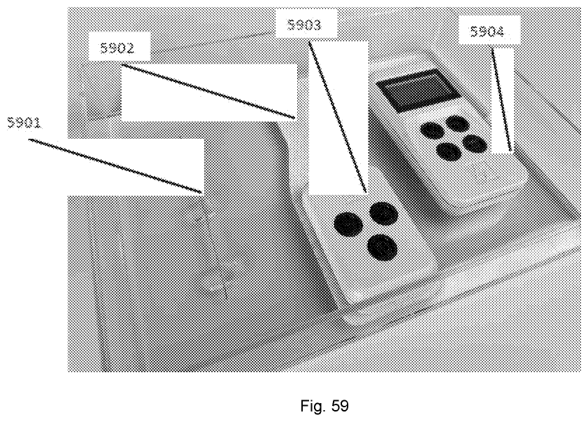

Referring to FIG. 56 and FIG. 57, multi-contact helical wire structure electrodes with standard ring contacts and implantable generator connections are illustrated. A multiple contact helical wire structure electrode contains ring contacts compatible with implantable pulse generator headers. Multiple of the multiple contact electrodes with standard ring connectors are connected to a multiple header implantable pulse generator, extending the number of contacts in the body on nerve or other tissue targets.

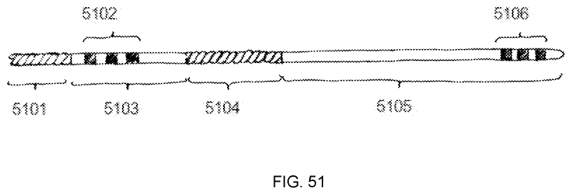

Referring to FIG. 51, a standard and injectable electrode hybrid configuration is illustrated. A leading helical wire structure of the electrode is made of conductive or non-conductive materials that allow for the formation of an anchoring bundle upon device placement. A central helical wire structure of the electrode, where connection wires from standard ring contacts are routed through an open helical coil structure, allows for the formation of an anchoring bundle upon device placement and replaces the need for the placement of strain relief loops.

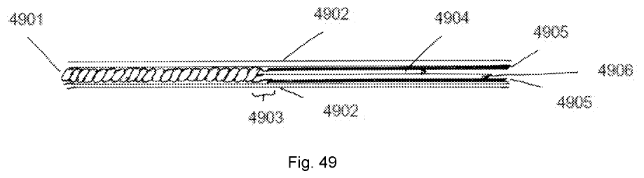

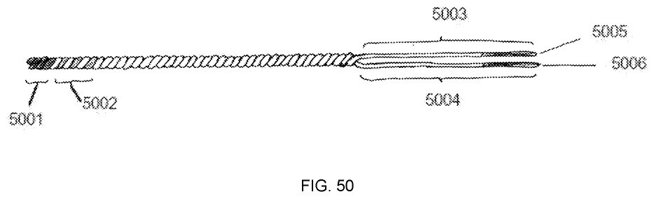

Referring to FIG. 50 and FIG. 49, split dual contact injectable electrodes and delivery systems are illustrated. A split dual contact injectable electrode includes helical wire structure contacts and split wire rope structures extending from the electrode body. The split wire rope structures include uncoated regions that provide exposed conductive surfaces for electrical connection. A delivery system for the split contact injectable electrode includes an external cannula for delivery, an internal delivery cannula having an inner diameter smaller than a diameter of the helical wire structure electrode but greater than two parallel rope wire structures, and split wire rope structures extending beyond the internal delivery cannula for interfacing during or after placement. The transition from helical to split structure has additional stabilization, adhesive, or heat shrink for the transition point. The internal delivery cannula has an inner diameter that is smaller than the diameter of the helical wire structure electrode but greater than two parallel rope wire structures, allowing for the advancement of the helical wire structure electrode while keeping the split ends free for interfacing either during placement or to allow for ease of interfacing post placement.

Referring to FIG. 58 and FIG. 59, interfacing solutions for injectable electrodes are illustrated including percutaneous and transcutaneous interfaces. A transcutaneous interface includes an injectable electrode interfaced with a dual contact skin adhesive conductive hydrogel patch onto which an external pulse generator connects. A percutaneous interface includes injectable electrodes that are either two monopolar electrodes or a single bipolar electrode. The electrodes are connected by way of conductive hook-and-loop connections that sufficiently secure the electrodes and provide a highly conductive electrical connection between the electrodes and the external pulse generator output via an adhesive patch adapter. The percutaneous system includes the capability to independently stimulate the electrodes versus a skin adhesive electrode to achieve a multi-polar setup, or stimulating between different contacts with 3, 4, 5, 6, 7, 8, 9, 10, 11, and 12 contacts either all separate, all multi-contact, or a mixture of both.

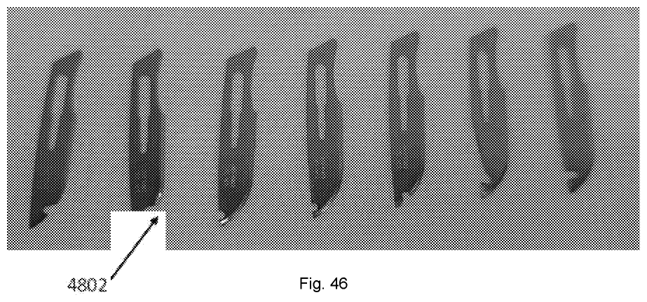

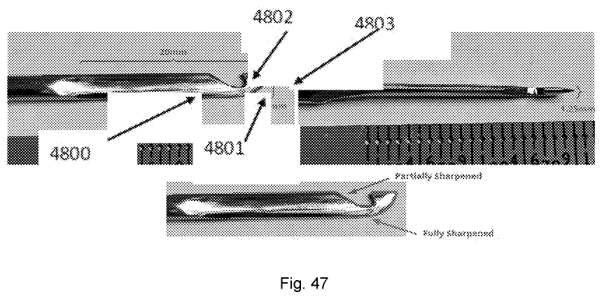

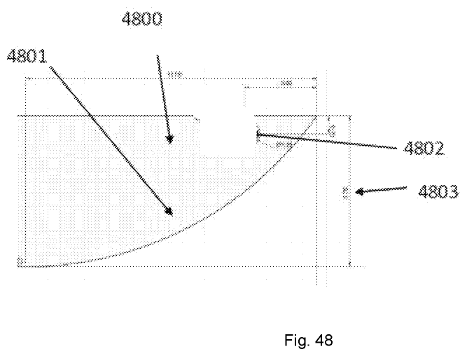

Referring to FIG. 46, FIG. 47, and FIG. 48, hook blade devices for percutaneous removal of injectable electrodes are illustrated. The hook blade allows for standard cut-down approaches and then facilitates device removal by introduction of a hook on the non-cutting edge of the blade. Using imaging, the alignment of the hook blade and confirmation of electrode engagement within the hook is procedurally confirmed. The hook blade includes a cutting edge curvature on an outer edge for performing primary skin and tissue cutting down to a target electrode, and a hook on a non-cutting edge of the blade configured to engage with and retrieve the electrode. The hook is large enough in diameter to allow for ease of hooking onto the electrode, and is at least 1.25 times the diameter of the target electrode and up to 10 times the diameter. The inner edge of the hook has a sharpness level that cuts tissue but does not cut the electrode. The outer edge is sharper than the inner edge in order to facilitate ease of tissue cutting down to the target. The outer edge does not easily cut through the electrode in a slicing motion, allowing the electrode to withstand 1 to 20 slices from the blade prior to introducing a potential fracture point.

Impedance scanning and/or piezoelectrically detection methods may be integrated into the hook blade as previously described. These may then provide the clinical user with the visual and/or audible ques to determine whether the hook blade has made contact with the implanted electrode.

The hook blade device may be electrically connected to an impedance analyzer that differentiates contact with tissue versus contact with an implanted electrode by changes in measured impedance with respect to a distal return (patch or needle) or circuit return included as a separate conductive feature on the face or edge of the blade in a region outside of the hook. The impedance analyzer may sweep between different frequencies (10 Hz, 100 Hz, 1 kHz, 10 kHz, 100 kHz, 1 MHz) for which the determined impedance and electrical impedance spectroscopy plot will differ between tissue only contact and contact with a conductive electrode. An audible and/or visual signal will then supplement the tactile feedback clinicians normally rely on when performing percutaneous surgical removal of electrodes.

In addition to or as an alternative to electrical impedance based detection, audible and ultrasound frequency based detection may be performed directly through the hook blade device. When the hook blade contacts the implanted electrode, the scraping of the blade against the metallic structure of the electrode produces high frequency oscillations that are either audibly perceived as scratching, or physically perceived as vibrations. Connecting a piezoelectric sensor (audible or ultrasound frequency ranges from 10 Hz to 15 MHz) directly to the blade provides the input data to a digital analyzer that determines whether contact has or has not been with an electrode. Audible and/or visual feedback is then provided to the clinical user.

Dimensional specifications for the hook blade include an entire blade length measured from base to the sharp tip, for example 37.1 mm for a #15 blade. The blade width is the maximum transverse dimension spanning from the spine to the farthest point of the cutting edge, for example 6.2 mm. The blade thickness is the cross-sectional thickness of the blade's material, for example 0.87 mm as per sheet metal thickness capabilities. The cutting edge cross-sectional length extends from the tip along the curved edge, for example 12 mm. The hook angle is the angular orientation of the hook modification relative to the blade's spine, for example 35 degrees. The hook diameter is the diameter of the circular cut forming the hook modification, for example 1.25 mm. The hook center to edge is the linear distance from the blade's tip to the center of the hook modification, for example 3.0 mm. The center of hook to spine is the perpendicular distance from the center of the hook circle to the blade's spine, for example 0.75 mm. A steeper hook angle less than 35 degrees creates a more aggressive hook but increases the risk of damaging surrounding tissue, while a shallower angle greater than 35 degrees results in smoother interaction with surrounding tissue but makes it probable that the electrode could slip.

In an overwound bipolar injectable electrode, two mandrels are used: one for a leading electrode contact and another for a remainder of the helical wire structure beyond a second contact tissue interface. In the region between the two mandrels, the coated wire structure of the leading electrode spans the distance of the second electrode contact and serves as the mandrel in that region. To achieve contacts beyond two in a bipolar configuration, the distance of the gap between the two mandrel setup is extended to accommodate additional leads. Following each additional contact, the lead of the previous contact becomes the core of the helical wire structure while all other leads before it return to being the externally wound leads to form the helical wire structures.

The following is a list of figures and elements included in this disclosure. FIG. 46 illustrates a top view of surgical blades modified to include a hook 4802. The hook 4802 is positioned along the non-cutting edge of each blade near the tip region and is designed to facilitate engagement with and retrieval of implanted electrodes during removal procedures.

FIG. 47 illustrates a prototype example of a hook blade 4800 for electrode removal. The hook blade 4800 includes a cutting edge curvature 4801 that extends along the outer edge of the hook blade 4800, providing the primary cutting surface for tissue incision during electrode removal procedures. The hook 4802 is formed near the tip of the hook blade 4800 on the non-cutting edge. A blade width 4803 is shown at the tip region near the hook 4802.

FIG. 48 illustrates a detailed view of the hook blade 4800 for electrode removal. The cutting edge curvature 4801 extends along the outer edge of the hook blade 4800 for performing incisions through skin and tissue to access a target electrode. The hook 4802 is positioned on the non-cutting edge of the hook blade 4800, opposite the cutting edge curvature 4801. The blade width 4803 indicates the transverse dimension of the hook blade 4800, spanning from the spine to the farthest point of the cutting edge curvature 4801.

FIG. 49 illustrates a side view of a delivery system for a split contact injectable electrode. The delivery system includes a dual contact injectable electrode with split end 4901 positioned within an external cannula 4902. A transition point 4903 is located where the helical structure transitions to the split wire rope configuration. Extending from the transition point 4903 are a contact 1 wire rope structure 4904 and a contact 2 wire rope structure 4906. An internal delivery cannula 4905 is positioned within the external cannula 4902 and has an inner diameter that is smaller than the diameter of the helical wire structure electrode but greater than two parallel rope wire structures. The contact 1 wire rope structure 4904 and the contact 2 wire rope structure 4906 extend beyond the internal delivery cannula 4905.

FIG. 50 illustrates a side view of a split dual contact injectable electrode. The split dual contact injectable electrode includes a helical wire structure contact 5001 and a helical wire structure contact 5002 positioned along the length of the electrode. Extending from the electrode structure is a split of contact 1 wire rope structure 5003 and a split of contact 2 wire rope structure 5004. An uncoated region of contact 1 wire rope structure 5005 is positioned along the split of contact 1 wire rope structure 5003, and an uncoated region of contact 2 wire rope structure 5006 is positioned along the split of contact 2 wire rope structure 5004. These uncoated regions provide exposed conductive surfaces for electrical connection.

FIG. 51 illustrates a side view of a standard and injectable electrode hybrid configuration. A leading helical wire structure 5101 is positioned at one end of the electrode. Adjacent to the leading helical wire structure 5101 are standard lead ring contact electrodes 5102. A standard lead section 5103 extends from the standard lead ring contact electrodes 5102 toward the central portion of the device. A central helical wire structure 5104 is positioned in the middle region of the electrode, where connection wires from the standard ring contacts are routed through an open helical coil structure. Extending from the central helical wire structure 5104 is another standard lead section 5105, which connects to standard lead ring contacts 5106 located at the opposite end of the device.

FIG. 52 illustrates a manufacturing sequence for a loopback electrode configuration. A proximal electrode 5201 is positioned as an uncoated region at each end of a rope wire structure, and an uncoated offset region 5202 is located near the middle of the rope wire structure. The rope wire structure is looped back upon itself at the uncoated offset region 5202, positioning the two proximal electrodes 5201 in an offset arrangement. The loopback rope wire structure is wound on a mandrel to create a helical wire loopback structure electrode with two stimulating proximal electrodes 1 at the proximal end and one distal electrode formed by the uncoated offset region 5202.

FIG. 53 illustrates a procedure for adding a crimp connection to a previously implanted electrode. A previously implanted helical wire structure 5301 is partially removed through an incision 5302 in skin 3. The helical wire structure 5301 is extended to create an elongated portion 5304 at the collector site. A cylindrical conductive contact 5305 is attached to the exposed and cut collector using either a crimped connection 5306 or a swaged connection 5307.

FIG. 54 illustrates a cross-section of a connection configuration before and after crimping. An electrode 5401 extends toward an implantable pulse generator 5403, with crimping 5402 forces being applied. The implantable pulse generator 5403 includes a solder pad 5404 positioned at the interface region. A secondary tube 5504 is associated with the implantable pulse generator 5403 assembly.

FIG. 55 illustrates discrete components of a crimp and swage based connection system. An electrode 5501 appears as an elongated wire rope structure. A primary tube 5502 is a cylindrical conductive connector positioned adjacent to the electrode 5501, having an inner diameter greater than the diameter of the electrode 5501. A secondary tube 5503 is positioned to the right of the primary tube 5502, featuring an inner diameter greater than that of the primary tube 5502. The secondary tube 5503 facilitates connection to the implantable pulse generator.

FIG. 56 illustrates an orthogonal view of a multiple contact helical wire structure electrode with a ring contact 5601 and an electrode 5602. The ring contact 5601 is positioned at one end and the electrode 5602 is positioned at the opposite end. The ring contact 5601 is configured to be compatible with implantable pulse generator headers.

FIG. 57 illustrates an implantable generator 5701 connected to a dual contact electrode 5702. The implantable generator 5701 appears as an oval-shaped device suitable for implantation within the body. The dual contact electrode 5702 extends from the implantable generator 5701 as an elongated helical wire structure with exposed contact regions for interfacing with tissue targets.

FIG. 58 illustrates a perspective view of a percutaneous injectable electrode system 5800. The percutaneous injectable electrode system 5800 includes a monopolar electrode 5801 and a monopolar electrode 5802 attached to a system 5804. A bipolar electrode 5803 is shown separately, demonstrating an alternative single electrode configuration. The system 5804 includes an adhesive patch adapter with an external pulse generator attached.

FIG. 59 illustrates a perspective view of a transcutaneously coupled injectable electrode system. An injectable electrode 5901 appears as an elongated transparent helical wire structure. A dual contact skin adhesive conductive hydrogel patch 5902 serves as an interface between the injectable electrode 5901 and external components. An external pulse generator 5903 is positioned on top of the dual contact skin adhesive conductive hydrogel patch 5902. A remote control 5904 provides user control functionality for the external pulse generator 5903.

FIG. 60 illustrates a perspective view of a multi-contact connector assembly. A first cylindrical contact 6001 is positioned at the proximal end of the assembly, followed by a second cylindrical contact 6002 in the middle position, and a third cylindrical contact 6003 at the distal end. Each of the first cylindrical contact 6001, the second cylindrical contact 6002, and the third cylindrical contact 6003 is separated from adjacent contacts by insulative material.

FIG. 53 illustrates a procedure for adding a connector to a previously implanted electrode. A previously implanted helical wire structure 5101 is partially removed through an incision 5102 made in skin 5103. The distal portion of the electrode is an elongated electrode 5104 at the collector site. A cylindrical conductive contact 5105 is attached to the exposed and cut collector using either a crimped contact 5106 or a swaged contact 5307.

FIG. 52 illustrates sequential views depicting fabrication of a loopback electrode structure. A proximal electrode 5201 is positioned as an uncoated region on each end of a rope wire structure, and an uncoated offset region 5202 is positioned near the middle of the structure. The rope wire structure is looped back upon itself with the proximal electrodes 5201 offset from each other. The loopback rope wire structure is wound on a mandrel to create a helical wire loopback structure electrode with two stimulating proximal electrodes 1 at the proximal end and one distal electrode formed by the uncoated offset region 5202.

Referring to FIG. 46, FIG. 47, and FIG. 48, a hook blade 4800 for percutaneous removal of an injectable electrode is illustrated. The hook blade 4800 allows for standard cut-down approaches and facilitates device removal by introduction of a hook 4802 on the non-cutting edge of the blade. FIG. 46 illustrates a top view of standard surgical blades modified to include the hook 4802, where the hook 4802 is positioned along the non-cutting edge of each blade near the tip region. FIG. 47 illustrates a prototype example of the hook blade 4800 design for electrode removal, showing a cutting edge curvature 4801 that extends along the outer edge of the hook blade 4800 and provides the primary cutting surface for tissue incision during electrode removal procedures. FIG. 48 illustrates a detailed view of the hook blade 4800, showing the cutting edge curvature 4801 extending along the outer edge for performing incisions through skin and tissue to access a target electrode, with the hook 4802 positioned on the non-cutting edge opposite the cutting edge curvature 4801. A blade width 4803 indicates the transverse dimension of the hook blade 4800, spanning from the spine to the farthest point of the cutting edge curvature 4801.

The hook blade 4800 includes two cutting edges with differential sharpness characteristics. The outer edge of the hook blade 4800 performs primary skin and tissue cutting down to the target electrode for removal. The inner edge of the hook 4802 is configured to engage with and retrieve the electrode during the removal process. The outer edge is sharper than the inner edge to facilitate ease of tissue cutting down to the target. The inner edge of the hook 4802 has a sharpness level that cuts tissue but does not cut the electrode. The outer edge does not easily cut through the electrode in a slicing motion, allowing the electrode to withstand 1 to 20 slices from the blade prior to introducing a potential fracture point in the electrode that could fail during the removal process.

The hook 4802 is large enough in diameter to allow for ease of hooking onto the electrode in a cutting motion. The hook 4802 has a diameter that is at least 1.25 times the diameter of the target electrode and up to 10 times the diameter, as larger sizes result in unnecessary tissue damage. Using imaging, the alignment of the hook blade 4800 and confirmation of electrode engagement within the hook 4802 is procedurally confirmed. The hook blade 4800 provides clinicians with a tool that combines cutting functionality with electrode engagement capability, enabling both tissue incision and electrode retrieval using a single instrument.

Referring to FIG. 47 and FIG. 48, the hook blade 4800 includes dimensional specifications that define the geometry and functional characteristics of the removal device. An entire blade length is measured from a base attachment area to a sharp tip of the hook blade 4800, and is for example 37.1 mm for a #15 blade configuration. The blade width 4803 is a maximum transverse dimension spanning from a spine of the hook blade 4800 to a farthest point of the cutting edge curvature 4801, and is for example 6.2 mm. A blade thickness is a cross-sectional thickness of the material of the hook blade 4800, and is for example 0.87 mm as per sheet metal thickness capabilities. A cutting edge cross-sectional length extends from the tip of the hook blade 4800 along the cutting edge curvature 4801, and is for example 12 mm.

A hook angle is an angular orientation of the hook 4802 relative to the spine of the hook blade 4800, and is for example 35 degrees. The hook angle influences engagement of the hook 4802 with a target electrode during removal procedures. A steeper hook angle less than 35 degrees creates a more aggressive hook 4802 configuration but increases the risk of damaging surrounding tissue. A shallower hook angle greater than 35 degrees results in smoother interaction with surrounding tissue but makes it probable that the electrode could slip from the hook 4802. The hook angle balances secure grip on the electrode with ease of maneuverability during the removal procedure.

A hook diameter is a diameter of a circular cut forming the hook 4802, and is for example 1.25 mm. The hook diameter determines the size of an opening of the hook 4802 and influences the ability of the hook 4802 to retrieve the target electrode. A hook center to edge distance is a linear distance from the tip of the hook blade 4800 to a center of the hook 4802, and is for example 3.0 mm. The hook center to edge distance positions the hook 4802 relative to a cutting surface of the hook blade 4800. A center of hook to spine distance is a perpendicular distance from the center of the hook 4802 to the spine of the hook blade 4800, and is for example 0.75 mm. The center of hook to spine distance affects structural integrity of the hook blade 4800 by determining an amount of material remaining along the spine.

Referring to FIG. 49, a delivery system for a split contact injectable electrode is illustrated. The delivery system includes the dual contact injectable electrode with split end 4901 positioned within the external cannula 4902. The external cannula 4902 serves as an outer delivery sheath for guiding the dual contact injectable electrode with split end 4901 during placement into tissue. The transition point 4903 is located where the helical structure transitions to the split wire rope configuration. The transition point 4903 includes additional stabilization features such as adhesive or heat shrink material to secure the transition from the helical structure to the split wire rope structures. Extending from the transition point 4903 are the contact 1 wire rope structure 4904 and the contact 2 wire rope structure 4906, which represent individual conductor leads of the split dual contact electrode. The internal delivery cannula 4905 is positioned within the external cannula 4902 and has an inner diameter that is smaller than a diameter of the helical wire structure electrode but greater than two parallel rope wire structures. This configuration of the internal delivery cannula 4905 allows for advancement of the helical wire structure electrode while keeping the split ends free for interfacing. The contact 1 wire rope structure 4904 and the contact 2 wire rope structure 4906 extend beyond the internal delivery cannula 4905, enabling connection to external components or pulse generators either during placement or post-placement.

Referring to FIG. 50, a split dual contact injectable electrode is illustrated. The split dual contact injectable electrode includes the helical wire structure contact 5001 and the helical wire structure contact 5002 positioned along the length of the electrode body. The helical wire structure contact 5001 and the helical wire structure contact 5002 serve as tissue interface contacts for delivering electrical stimulation to target tissue. The helical wire structure of the electrode allows for the formation of an anchoring bundle upon device placement, and when inserted into the body the proximal end does not protrude, making the device less obtrusive to the patient. The split dual contact injectable electrode is self-anchoring and includes both helical coils and non-helical coils and structures that carry an electrical signal from a proximal end to a distal end.

Extending from the electrode body is the split of contact 1 wire rope structure 5003 and the split of contact 2 wire rope structure 5004. The split of contact 1 wire rope structure 5003 and the split of contact 2 wire rope structure 5004 represent individual wire rope structures that separate from the helical configuration to facilitate interfacing with external components or pulse generators. The split configuration of the wire rope structures allows for advancement of the helical wire structure electrode during delivery while keeping the split ends accessible for connection either during placement or post-placement procedures.

The uncoated region of contact 1 wire rope structure 5005 is positioned along the split of contact 1 wire rope structure 5003, and the uncoated region of contact 2 wire rope structure 5006 is positioned along the split of contact 2 wire rope structure 5004. The uncoated region of contact 1 wire rope structure 5005 and the uncoated region of contact 2 wire rope structure 5006 provide exposed conductive surfaces that enable electrical connection and interfacing capabilities. The uncoated regions are areas where insulative coating has been removed to expose the underlying conductive material, allowing for direct electrical contact with external pulse generators or other stimulation equipment.

Referring to FIG. 51, a standard and injectable electrode hybrid configuration is illustrated. The standard and injectable electrode hybrid configuration combines helical wire structure elements with standard lead components to provide both anchoring capability and compatibility with conventional pulse generator connection systems. The leading helical wire structure 5101 is positioned at one end of the electrode and is made of conductive or non-conductive materials that allow for the formation of an anchoring bundle upon device placement. The leading helical wire structure 5101 provides mechanical stabilization within tissue when the electrode is deployed.

Adjacent to the leading helical wire structure 5101, the standard lead ring contact electrodes 5102 are positioned along the electrode body. The standard lead ring contact electrodes 5102 provide electrical contact points for interfacing with tissue targets and delivering electrical stimulation. The standard lead section 5103 extends from the standard lead ring contact electrodes 5102 toward a central portion of the device, providing a connection pathway between the ring contact electrodes and the central region of the electrode.

The central helical wire structure 5104 is positioned in a middle region of the electrode. Connection wires from the standard lead ring contact electrodes 5102 are routed through an open helical coil structure of the central helical wire structure 5104. The central helical wire structure 5104 allows for the formation of an anchoring bundle upon device placement and replaces the need for placement of strain relief loops. The open helical coil structure of the central helical wire structure 5104 accommodates the routing of connection wires while maintaining the anchoring functionality of the helical configuration.

The standard lead section 5105 extends from the central helical wire structure 5104 toward an opposite end of the device. The standard lead section 5105 connects to the standard lead ring contacts 5106 located at the opposite end of the electrode from the leading helical wire structure 5101. The standard lead ring contacts 5106 are configured to connect to an implantable pulse generator or an external pulse generator, providing the electrical interface between the electrode and stimulation equipment. The hybrid configuration of the electrode integrates helical wire structure elements for anchoring with standard lead components for pulse generator compatibility.

Referring to FIG. 52, a manufacturing sequence for a loopback electrode configuration is illustrated. The loopback electrode configuration produces a helical wire structure electrode with two exposed electrodes at a proximal end configured for placement on an electrical stimulation target and one exposed electrode at a distal location configured to serve as a collector. The helical wire structure electrode is fabricated from a single wire rope structure. The proximal electrode 5201 is positioned as an uncoated region at each end of the rope wire structure, and the uncoated offset region 5202 is located near the middle of the rope wire structure. The uncoated ends of the rope wire structure form the proximal electrodes, and the uncoated offset region 5202 forms the distal electrode.

The rope wire structure is folded back upon itself at the uncoated offset region 5202 near the middle of the rope to produce two parallel ropes. The proximal electrode 5201 at each end is offset from the other proximal electrode 5201 so as to not directly overlap when the structure is folded. The resulting U-shaped parallel rope wire structure is wound on a mandrel to create a helical wire loopback structure electrode. The helical wire loopback structure electrode includes a stimulating proximal electrode 1 at each end of the proximal region, providing two stimulating proximal electrodes 1 at the proximal end, and one distal electrode formed by the uncoated offset region 5202 that serves as the collector.

The loopback electrode configuration is reversible. In a reversed configuration, the loopback central electrode formed by the uncoated offset region 5202 is placed on the stimulation target proximally. This reversed configuration produces a helical wire loopback structure electrode with one proximal electrode and two distal electrodes instead of two proximal electrodes and one distal electrode. The reversed configuration allows for different stimulation and collection arrangements depending on the clinical application.

Two or more helical wire loopback structures are combined to create multiple loopback electrodes. The loopback rope wire structures are offset at a predetermined distance from each other and placed in parallel. The parallel loopback rope wire structures are then wound together around a mandrel. This manufacturing approach produces a multi-loopback electrode configuration with multiple stimulating and collecting electrode contacts integrated into a single helical wire structure.

Referring to FIG. 53, a procedure for adding a crimp connection to a previously implanted electrode is illustrated. A previously implanted helical wire structure 5301 is partially removed through an incision 5302 in skin 3. The partial extraction procedure involves accessing the distal component of the implanted helical wire structure 5301 and extending the structure back to a non-helical rope wire structure configuration. The helical wire structure 5301 is extended to create an elongated portion 5304 at the collector site. The elongated portion 5304 represents the electrode in the rope wire structure configuration after partial extraction from the body.

The partially elongated electrode end in the rope wire structure configuration is cut to length to prepare the electrode for connector attachment. A cylindrical conductive contact 5305 is applied by threading the wire rope structure into the cylinder. The cylindrical conductive contact 5305 is secured to the electrode using either a crimping force or distance, or alternatively using a predetermined swaging diameter reduction to mechanically and electrically secure the cylinder to the electrode.

In a first connection method, the cylindrical conductive contact 5305 is attached to the exposed and cut collector using a crimped connection 5306. The crimped connection 5306 is formed by applying a compressive crimping force to the cylindrical conductive contact 5305, which reduces the inner diameter of the cylindrical conductive contact 5305 and creates mechanical engagement with the wire rope structure. The crimping process establishes both mechanical and electrical connection between the cylindrical conductive contact 5305 and the electrode.

In a second connection method, the cylindrical conductive contact 5305 is secured to the exposed and cut collector using a swaged connection 5307. The swaged connection 5307 is formed by applying a predetermined swaging diameter reduction to the cylindrical conductive contact 5305. The swaging process reduces the diameter of the cylindrical conductive contact 5305 in a controlled manner to achieve both mechanical and electrical connection with the wire rope structure of the electrode.

The procedure enables conversion of the previously implanted helical wire structure 5301 into an electrode configuration that interfaces with a socket connector of an implantable pulse generator without requiring complete removal of the electrode from the body. The proximal end of the electrode remains unmoved at the original implantation site while the distal end receives the cylindrical conductive contact 5305 through either the crimped connection 5306 or the swaged connection 5307.

Referring to FIG. 54, a cross-section of a connection configuration before and after crimping is illustrated. The electrode 5401 extends toward the implantable pulse generator 5403, with the crimping 5402 forces being applied from above and below to reduce the diameter of a cylindrical contact surrounding the electrode 5401. The implantable pulse generator 5403 includes the solder pad 5404 positioned at an interface region where electrical connection is established. The secondary tube 5504 is associated with the implantable pulse generator 5403 assembly and has an inner diameter greater than that of a primary tube, facilitating connection between the electrode 5401 and the implantable pulse generator 5403.

The crimping 5402 process mechanically and electrically secures the electrode 5401 to the implantable pulse generator 5403 through diameter reduction of the cylindrical contact. The cylindrical conductive contact is secured to the electrode 5401 using either a crimping force or distance, or alternatively using a predetermined swaging diameter reduction to mechanically and electrically secure the cylinder to the electrode 5401. In the pre-crimping configuration shown in the upper portion of FIG. 54, the electrode 5401 is positioned within the cylindrical contact with the crimping 5402 forces indicated by directional arrows. In the post-crimping configuration shown in the lower portion of FIG. 54, the electrode 5401 is mechanically and electrically secured to the implantable pulse generator 5403 through the completed crimping 5402 process.

A leading helical wire structure of the electrode 5401 is made of conductive or non-conductive materials that allow for the formation of an anchoring bundle upon device placement. Multiple contact electrodes with standard ring connectors are connected to a multiple header implantable pulse generator, extending the number of contacts in the body on nerve or other tissue targets. The solder pad 5404 provides a surface for establishing electrical continuity between the electrode 5401 and circuitry of the implantable pulse generator 5403. The secondary tube 5504 accommodates the crimped connection and provides structural support for the electrode-to-generator interface.

Referring to FIG. 54, a cross-section of a connection configuration before and after crimping is illustrated. The electrode 5401 extends toward the implantable pulse generator 5403, with the crimping 5402 forces being applied from above and below to reduce the diameter of a cylindrical contact surrounding the electrode 5401. The implantable pulse generator 5403 includes the solder pad 5404 positioned at an interface region where electrical connection is established. The secondary tube 5504 is associated with the implantable pulse generator 5403 assembly and has an inner diameter greater than that of a primary tube, facilitating connection between the electrode 5401 and the implantable pulse generator 5403.

The crimping 5402 process mechanically and electrically secures the electrode 5401 to the implantable pulse generator 5403 through diameter reduction of the cylindrical contact. The cylindrical conductive contact is secured to the electrode 5401 using either a crimping force or distance, or alternatively using a predetermined swaging diameter reduction to mechanically and electrically secure the cylinder to the electrode 5401. In the pre-crimping configuration shown in the upper portion of FIG. 54, the electrode 5401 is positioned within the cylindrical contact with the crimping 5402 forces indicated by directional arrows. In the post-crimping configuration shown in the lower portion of FIG. 54, the electrode 5401 is mechanically and electrically secured to the implantable pulse generator 5403 through the completed crimping 5402 process.

A leading helical wire structure of the electrode 5401 is made of conductive or non-conductive materials that allow for the formation of an anchoring bundle upon device placement. Multiple contact electrodes with standard ring connectors are connected to a multiple header implantable pulse generator, extending the number of contacts in the body on nerve or other tissue targets. The solder pad 5404 provides a surface for establishing electrical continuity between the electrode 5401 and circuitry of the implantable pulse generator 5403. The secondary tube 5504 accommodates the crimped connection and provides structural support for the electrode-to-generator interface.

Referring to FIG. 56, a multiple contact helical wire structure electrode with standard ring contacts is illustrated. The multiple contact helical wire structure electrode includes a ring contact 5601 positioned at one end and an electrode 5602 positioned at the opposite end. The ring contact 5601 is configured to be compatible with implantable pulse generator headers, allowing the multiple contact helical wire structure electrode to interface with standard electrical stimulation systems. The electrode 5602 serves as a tissue interface contact for delivering electrical stimulation to target tissue.

Individual conductors of the multiple contact helical wire structure electrode are connected to each individual cylindrical contact through welding, brazing, or using a conductive adhesive. These connection methods establish both mechanical and electrical connections between the conductors and the ring contact 5601. The welding process fuses the conductor material to the contact material, the brazing process joins the conductor to the contact using a filler metal, and the conductive adhesive bonds the conductor to the contact while providing an electrically conductive pathway.

A leading helical wire structure of the electrode 5602 is made of conductive or non-conductive materials that allow for the formation of an anchoring bundle upon device placement. The anchoring bundle provides mechanical stabilization within tissue when the multiple contact helical wire structure electrode is deployed. The helical configuration of the leading wire structure enables the electrode 5602 to form the anchoring bundle through the natural coiling behavior of the helical structure upon release from a delivery device.

Multiple of the multiple contact helical wire structure electrodes with standard ring connectors are connected to a multiple header implantable pulse generator, extending the number of contacts in the body on nerve or other tissue targets. This configuration allows a single implantable pulse generator to interface with multiple electrode contacts distributed across different anatomical locations. The ring contact 5601 of each multiple contact helical wire structure electrode engages with a corresponding header connection on the implantable pulse generator, establishing independent electrical pathways for each electrode contact.

Referring to FIG. 57, the implantable generator 5701 connected to the dual contact electrode 5702 is illustrated in both connected and loose configurations. The implantable generator 5701 appears as an oval-shaped device with a smooth exterior surface suitable for implantation within the body. The dual contact electrode 5702 extends from the implantable generator 5701 as an elongated helical wire structure with exposed contact regions for interfacing with tissue targets. In the connected configuration shown in the upper portion of FIG. 57, the dual contact electrode 5702 is attached to the implantable generator 5701, establishing an electrical pathway for delivering stimulation signals. In the loose configuration shown in the lower portion of FIG. 57, the dual contact electrode 5702 is positioned separately from the implantable generator 5701, demonstrating the relationship between the components before connection.

Multi-contact injectable electrodes or one or more monopolar injectable electrodes with or without integrated standard ring connectors are connected to an implantable pulse generator for delivering electrical stimulation to target tissues within the body. The connection between the dual contact electrode 5702 and the implantable generator 5701 enables transmission of electrical signals from the implantable generator 5701 through the dual contact electrode 5702 to nerve or other tissue targets. The implantable generator 5701 contains circuitry that generates electrical pulses at specified parameters including amplitude, frequency, and pulse width. The dual contact electrode 5702 delivers the electrical pulses to the target tissue through the exposed contact regions positioned along the helical wire structure.

The implantable generator 5701 includes a header region configured to receive and secure the dual contact electrode 5702. The header region contains socket connectors that interface with corresponding connector elements on the dual contact electrode 5702. When the dual contact electrode 5702 is inserted into the header region of the implantable generator 5701, mechanical and electrical connections are established between the components. The mechanical connection secures the dual contact electrode 5702 to the implantable generator 5701 to prevent disconnection during patient movement or activity. The electrical connection provides a conductive pathway for stimulation signals to travel from the implantable generator 5701 to the tissue interface contacts of the dual contact electrode 5702.

Multiple of the dual contact electrodes 5702 with standard ring connectors are connected to a multiple header implantable pulse generator, extending the number of contacts in the body on nerve or other tissue targets. This configuration allows a single implantable pulse generator to interface with multiple electrode contacts distributed across different anatomical locations. Each dual contact electrode 5702 engages with a corresponding header connection on the implantable pulse generator, establishing independent electrical pathways for each electrode contact. The multiple header configuration of the implantable pulse generator accommodates connection of several electrodes simultaneously, enabling stimulation of multiple tissue targets from a single implanted power source.

Referring to FIG. 58, the percutaneous injectable electrode system 5800 is illustrated. The percutaneous injectable electrode system 5800 provides a percutaneous coupling interface for injectable electrodes to connect with external stimulation equipment. The percutaneous injectable electrode system 5800 includes multiple electrode configurations that interface with tissue targets for delivering electrical stimulation.

The percutaneous injectable electrode system 5800 comprises the monopolar electrode 5801 and the monopolar electrode 5802, which are attached to the system 5804. The monopolar electrode 5801 and the monopolar electrode 5802 represent two separate injectable electrodes that are connected to the system 5804 for delivering electrical stimulation to target tissues. The system 5804 includes an adhesive patch adapter with an external pulse generator attached. The adhesive patch adapter of the system 5804 provides a mounting surface that adheres to skin and facilitates electrical connection between the electrodes and the external pulse generator.

The bipolar electrode 5803 is shown separately in FIG. 58, demonstrating an alternative single electrode configuration. The bipolar electrode 5803 provides a self-contained electrode arrangement with two contacts integrated into a single structure. The bipolar electrode 5803 is configured to connect to the system 5804 as an alternative to the dual monopolar electrode configuration comprising the monopolar electrode 5801 and the monopolar electrode 5802.

The monopolar electrode 5801, the monopolar electrode 5802, and the bipolar electrode 5803 are connected to the system 5804 by way of conductive hook-and-loop connections. The conductive hook-and-loop connections sufficiently secure the electrodes and provide a highly conductive electrical connection between the electrodes and the external pulse generator output via the adhesive patch adapter of the system 5804. The conductive hook-and-loop connections establish both mechanical attachment and electrical continuity between the injectable electrodes and the external pulse generator.

The percutaneous injectable electrode system 5800 includes the capability to independently stimulate the electrodes versus a skin adhesive electrode to achieve a multi-polar setup. The percutaneous injectable electrode system 5800 is configured for stimulating between different contacts with 3, 4, 5, 6, 7, 8, 9, 10, 11, and 12 contacts. The contacts are configured as all separate contacts, all multi-contact configurations, or a mixture of both separate and multi-contact configurations. This multi-polar capability enables the percutaneous injectable electrode system 5800 to deliver stimulation across multiple contact sites with independent control of each contact or groups of contacts.

Referring to FIG. 59, a transcutaneously coupled injectable electrode system is illustrated. The transcutaneously coupled injectable electrode system provides a non-invasive interface between an implanted electrode and external stimulation equipment through transcutaneous electrical coupling. The injectable electrode 5901 appears as an elongated helical wire structure configured for implantation within tissue. The injectable electrode 5901 includes both helical coils and non-helical coils and structures that carry an electrical signal from a proximal end to a distal end. The injectable electrode 5901 is self-anchoring, and when inserted into the body the proximal end does not protrude, making the device less obtrusive to the patient. A time of deployment of the injectable electrode 5901 is between 5 and 30 minutes, wherein a preferred deployment time is 15 minutes.

The dual contact skin adhesive conductive hydrogel patch 5902 serves as an interface between the injectable electrode 5901 and external components. The dual contact skin adhesive conductive hydrogel patch 5902 is configured to adhere to skin and provide transcutaneous electrical coupling with the injectable electrode 5901 positioned beneath the skin surface. The dual contact skin adhesive conductive hydrogel patch 5902 includes conductive hydrogel material that facilitates electrical signal transmission between the injectable electrode 5901 and external stimulation equipment.

The external pulse generator 5903 is positioned on top of the dual contact skin adhesive conductive hydrogel patch 5902 and connects to the dual contact skin adhesive conductive hydrogel patch 5902 to deliver electrical stimulation signals. The external pulse generator 5903 contains circuitry that generates electrical pulses at specified parameters including amplitude, frequency, and pulse width. The external pulse generator 5903 features a display screen on an upper surface for providing visual feedback to a user. The electrical stimulation signals generated by the external pulse generator 5903 are transmitted through the dual contact skin adhesive conductive hydrogel patch 5902 and coupled transcutaneously to the injectable electrode 5901 for delivery to target tissue.

The remote control 5904 provides user control functionality for the external pulse generator 5903. The remote control 5904 includes multiple buttons arranged on a front surface for operating the transcutaneously coupled injectable electrode system. The remote control 5904 enables a user to adjust stimulation parameters and control operation of the external pulse generator 5903 without direct interaction with the external pulse generator 5903. In some configurations, an application on a smartphone performs the same functions as the remote control 5904.

Referring to FIG. 60, a multi-contact connector assembly is illustrated. The multi-contact connector assembly includes the first cylindrical contact 6001 positioned at a proximal end of the assembly, the second cylindrical contact 6002 positioned in a middle position, and the third cylindrical contact 6003 positioned at a distal end. The first cylindrical contact 6001, the second cylindrical contact 6002, and the third cylindrical contact 6003 are arranged in a linear configuration along a common axis.

Insulative material is positioned between each of the first cylindrical contact 6001, the second cylindrical contact 6002, and the third cylindrical contact 6003. The insulative material separates adjacent cylindrical contacts from each other to prevent electrical shorting between the contacts. The insulative material partially extends into a remainder of a helical wire structure connected to the multi-contact connector assembly. This extension of the insulative material into the helical wire structure provides electrical isolation between individual conductors along a length of the electrode beyond the connector region.

Individual conductors of a multi-contact helical wire structure electrode are connected to each of the first cylindrical contact 6001, the second cylindrical contact 6002, and the third cylindrical contact 6003 through welding, brazing, or using a conductive adhesive. Welding fuses conductor material directly to contact material to establish both mechanical and electrical connections. Brazing joins conductors to contacts using a filler metal. Conductive adhesive bonds conductors to contacts while providing an electrically conductive pathway between the conductor and the contact.

The multi-contact connector assembly is configured to be placed inside a socket connector of an electrical stimulator or an implantable pulse generator. The multi-contact connector assembly provides multiple independent electrical pathways for multi-contact electrode configurations. The multi-contact connector assembly is used for electrical stimulation applications or other applications such as radiofrequency ablation.

Referring to FIG. 53, a procedure for adding a connector to a previously implanted electrode without complete removal from a body is illustrated. A helical wire structure 5301 that has been previously implanted is partially extracted through an incision 5302 made in skin 5303. The partial extraction extends the helical wire structure 5301 back to a non-helical rope wire structure configuration, creating an elongated electrode 5304 at a collector site. The proximal end of the helical wire structure 5301 remains unmoved at the stimulation target during this procedure.

The elongated electrode 5304 in the rope wire structure configuration is cut to length to prepare the electrode for connector attachment. A cylindrical conductive contact 5305 is applied by threading the wire rope structure into the cylinder. The cylindrical conductive contact 5305 is secured to the elongated electrode 5304 using either a crimping force or distance, or alternatively using a predetermined swaging diameter reduction to mechanically and electrically secure the cylinder to the electrode.

In a first connection method, the cylindrical conductive contact 5305 is attached to the exposed and cut collector using a crimped contact 5306. The crimped contact 5306 is formed by applying a compressive crimping force to the cylindrical conductive contact 5305, which reduces the inner diameter of the cylindrical conductive contact 5305 and creates mechanical engagement with the wire rope structure. The crimping process establishes both mechanical and electrical connection between the cylindrical conductive contact 5305 and the elongated electrode 5304.

In a second connection method, the cylindrical conductive contact 5305 is secured to the exposed and cut collector using a swaged contact 5307. The swaged contact 5307 is formed by applying a predetermined swaging diameter reduction to the cylindrical conductive contact 5305. The swaging process reduces the diameter of the cylindrical conductive contact 5305 in a controlled manner to achieve both mechanical and electrical connection with the wire rope structure of the elongated electrode 5304.

Individual conductors of a multiple contact helical wire structure electrode are connected to each individual cylindrical contact through welding, brazing, or using a conductive adhesive. Welding fuses conductor material directly to contact material to establish both mechanical and electrical connections. Brazing joins conductors to contacts using a filler metal. Conductive adhesive bonds conductors to contacts while providing an electrically conductive pathway between the conductor and the contact.

Insulative material separating cylindrical conductive contacts partially extends into a remainder of the helical wire structure 5301. This extension of the insulative material into the helical wire structure 5301 provides electrical isolation between individual conductors along a length of the electrode beyond the connector region.

The procedure enables conversion of the previously implanted helical wire structure 5301 into an electrode configuration that interfaces with a socket connector of an implantable pulse generator without requiring complete removal of the electrode from the body. The proximal end of the electrode remains at the original implantation site at the stimulation target while the distal end receives the cylindrical conductive contact 5305 through either the crimped contact 5306 or the swaged contact 5307.

Referring to FIG. 52, sequential views depicting fabrication of a loopback electrode structure are illustrated. The fabrication process produces a helical wire structure electrode with two exposed electrodes at a proximal end configured for placement on an electrical stimulation target and one exposed electrode at a distal location configured to serve as a collector. The helical wire structure electrode is fabricated from a single wire rope structure.

In a first stage of fabrication shown in the top view of FIG. 52, a rope wire structure includes a proximal electrode 5201 positioned as an uncoated region at each end of the rope wire structure. An uncoated offset region 5202 is positioned near the middle of the rope wire structure. The proximal electrode 5201 at each end and the uncoated offset region 5202 represent exposed conductive areas where insulative coating has been removed to allow electrical contact with tissue. The uncoated ends of the rope wire structure form the proximal electrodes, and the uncoated offset region 5202 forms a distal electrode.

In a second stage of fabrication shown in the middle view of FIG. 52, the rope wire structure is looped back upon itself at the uncoated offset region 5202 near the middle of the rope to produce two parallel ropes. The proximal electrode 5201 at each end is offset from the other proximal electrode 5201 so as to not directly overlap when the structure is folded. This folding creates a U-shaped parallel rope wire configuration with the two proximal electrodes 5201 positioned in an offset arrangement.

In a third stage of fabrication shown in the bottom view of FIG. 52, the loopback rope wire structure is wound on a mandrel to create a helical wire loopback structure electrode. The resulting helical wire loopback structure electrode includes two stimulating proximal electrodes 1 at the proximal end of the device, which are configured for placement on an electrical stimulation target, and one distal electrode formed by the uncoated offset region 5202 that serves as the collector.

The loopback electrode configuration is reversible. In a reversed configuration, a loopback central electrode formed by the uncoated offset region 5202 is placed on the stimulation target proximally. This reversed configuration produces a helical wire loopback structure electrode with one proximal electrode and two distal electrodes instead of two proximal electrodes and one distal electrode. The reversed configuration allows for different stimulation and collection arrangements depending on the clinical application.

Two or more helical wire loopback structures are combined to create multiple loopback electrodes. The loopback rope wire structures are offset at a predetermined distance from each other and placed in parallel. The parallel loopback rope wire structures are then wound together around a mandrel. This manufacturing approach produces a multi-loopback electrode configuration with multiple stimulating and collecting electrode contacts integrated into a single helical wire structure.

A leading helical wire structure of the electrode is made of conductive or non-conductive materials that allow for the formation of an anchoring bundle upon device placement. The anchoring bundle provides mechanical stabilization within tissue when the helical wire loopback structure electrode is deployed. The helical configuration of the leading wire structure enables the electrode to form the anchoring bundle through the natural coiling behavior of the helical structure upon release from a delivery device.

Referring to FIG. 49, FIG. 50, FIG. 51, FIG. 52, FIG. 53, FIG. 56 and FIG. 60, manufacturing methods for multi-contact injectable electrodes are described. Manufacturing procedures for producing multiple contact injectable electrodes include approaches where contact leads of multi-contact injectable helical wire structure electrodes serve as an inner core of the electrode that is normally hollow. A hollow structure of an open coil helical wire structure electrode is achieved by use of a winding mandrel, whereas an overwound approach relies on the mandrel in a region of the helical wire structure that is beyond electrode contact regions that interface with a tissue target.

In an overwound dual contact injectable electrode configuration, two mandrels are used during manufacturing. A first mandrel serves a leading electrode contact, and a second mandrel serves a remainder of the helical wire structure beyond a second contact tissue interface. In a region between the two mandrels, a coated wire structure of the leading electrode spans a distance of the second electrode contact and serves as the mandrel in that region. The coated wire structure of the leading electrode provides structural support for winding the helical configuration in the inter-mandrel region while maintaining electrical isolation between the contact regions.

To achieve contacts beyond two in a bipolar configuration, a distance of a gap between the two mandrels is extended to accommodate additional leads. The extended gap distance provides space for additional electrode contacts to be formed along the length of the helical wire structure. Following each additional contact, a lead of a previous contact becomes a core of the helical wire structure while all other leads before the previous contact return to being externally wound leads to form the helical wire structures. This sequential lead core arrangement allows for the production of multi-contact injectable electrodes with three, four, five, or more contacts integrated into a single helical wire structure configuration.

Referring to FIG. 60, the first cylindrical contact 6001, the second cylindrical contact 6002, and the third cylindrical contact 6003 are connected to individual conductors of the multi-contact helical wire structure electrode through welding, brazing, or using a conductive adhesive. The insulative material separating the first cylindrical contact 6001, the second cylindrical contact 6002, and the third cylindrical contact 6003 extends into a remainder of the helical wire structure to provide electrical isolation between individual conductors along a length of the electrode.

Referring to FIG. 56, the ring contact 5601 and the electrode 5602 are positioned at opposite ends of a multiple contact helical wire structure electrode. The ring contact 5601 is configured to be compatible with implantable pulse generator headers, allowing the multiple contact helical wire structure electrode manufactured using the overwound approach to interface with standard electrical stimulation systems.

Referring to FIG. 50, the helical wire structure contact 5001 and the helical wire structure contact 5002 are positioned along a length of a split dual contact injectable electrode. The split of contact 1 wire rope structure 5003 and the split of contact 2 wire rope structure 5004 extend from the electrode body to facilitate interfacing with external components or pulse generators. The uncoated region of contact 1 wire rope structure 5005 and the uncoated region of contact 2 wire rope structure 5006 provide exposed conductive surfaces for electrical connection.

Referring to FIG. 49, the dual contact injectable electrode with split end 4901 is positioned within the external cannula 4902 during delivery. The transition point 4903 is located where the helical structure transitions to the split wire rope configuration. The contact 1 wire rope structure 4904 and the contact 2 wire rope structure 4906 extend beyond the internal delivery cannula 4905, enabling connection to external components or pulse generators either during placement or post-placement.

Referring to FIG. 51, a standard and injectable electrode hybrid configuration combines helical wire structure elements with standard lead components. A leading helical wire structure 5101 is positioned at one end of the electrode and allows for formation of an anchoring bundle upon device placement. Standard lead ring contact electrodes 5102 are positioned adjacent to the leading helical wire structure 5101. A standard lead section 5103 extends from the standard lead ring contact electrodes 5102 toward a central portion of the device. A central helical wire structure 5104 is positioned in a middle region of the electrode, where connection wires from the standard ring contacts are routed through an open helical coil structure. A standard lead section 5105 extends from the central helical wire structure 5104 toward an opposite end of the device and connects to standard lead ring contacts 5106.

Referring to FIG. 53, the helical wire structure 5301 and the helical wire structure 5301 are partially extracted through the incision 5302 and the incision 5302 in the skin 5303 to create the elongated portion 5304 and the elongated electrode 5304 at a collector site. The cylindrical conductive contact 5305 and the cylindrical conductive contact 5305 are applied by threading a wire rope structure into a cylinder and securing the connection using either a crimping force or a swaging diameter reduction.

Referring to FIG. 52, loopback electrode configurations are manufactured by folding a rope wire structure back upon itself at an uncoated offset region near a middle of the rope to produce two parallel ropes. Proximal electrodes at each end are offset from each other so as not to directly overlap. The resulting U-shaped parallel rope wire structure is wound on a mandrel to create a helical wire loopback structure electrode. Two or more helical wire loopback structures are combined by offsetting the loopback rope wire structures at a predetermined distance, placing the loopback rope wire structures in parallel, and winding the parallel structure around a mandrel to create multiple loopback electrodes.

Referring to FIG. 52, FIG. 50, FIG. 51 and FIG. 53, the electrode systems described herein exhibit deployment and self-anchoring characteristics that facilitate implantation and patient comfort. The time of deployment of the electrode is between 5 and 30 minutes, wherein a preferred deployment time is 15 minutes. This deployment time range accommodates various clinical scenarios and anatomical targets while providing sufficient time for proper electrode positioning and anchoring bundle formation.

The electrode systems are self-anchoring, and when inserted into the body the proximal end does not protrude from the body. This configuration makes the device less obtrusive to the patient following implantation. The self-anchoring characteristic is achieved through the helical wire structure configuration, which forms an anchoring bundle upon device placement. The leading helical wire structure 5101 and the central helical wire structure 5104 shown in FIG. 51 allow for the formation of anchoring bundles that provide mechanical stabilization within tissue. Similarly, the helical wire structure 5301 shown in FIG. 53 and the helical wire structure 5301 shown in FIG. 53 demonstrate the self-anchoring configuration where the proximal end remains at the stimulation target without protruding from the body surface.

The electrode systems include both helical coils and non-helical coils and structures that carry an electrical signal from a proximal end to a distal end. The proximal end is in communication with an electrical current generator or voltage generator, and the distal end is implanted in a body of a patient or animal. Referring to FIG. 50, the helical wire structure contact 5001 and the helical wire structure contact 5002 represent helical coil configurations, while the split of contact 1 wire rope structure 5003 and the split of contact 2 wire rope structure 5004 represent non-helical structures extending from the electrode body. The combination of helical and non-helical structures accommodates various application requirements including electrical stimulation delivery, connector interfacing, and tissue anchoring.

Referring to FIG. 52, the proximal electrode 5201 and the proximal electrode 5201 are positioned as uncoated regions at each end of a rope wire structure, and the uncoated offset region 5202 and the uncoated offset region 5202 are located near the middle of the rope wire structure. When the loopback rope wire structure is wound on a mandrel, the resulting helical wire loopback structure electrode includes the stimulating proximal electrode 1 at the proximal end. The helical configuration enables the electrode to form an anchoring bundle through the natural coiling behavior of the helical structure upon release from a delivery device, contributing to the self-anchoring characteristic of the electrode systems.

The electrode systems described herein operate as a comprehensive ecosystem where loopback electrode configurations, connector systems, interfacing solutions, manufacturing methods, and removal devices interact to provide a complete solution for electrical stimulation applications. Referring to FIG. 52 and FIG. 52, the loopback electrode configuration produces a helical wire structure electrode with two exposed electrodes at a proximal end and one exposed electrode at a distal location. The loopback electrode configuration is reversible so that the loopback central electrode is placed on the stimulation target proximally, producing a helical wire loopback structure electrode with one proximal electrode and two distal electrodes instead of two proximal electrodes and one distal electrode. Two or more helical wire loopback structures are combined by offsetting the loopback rope wire structures at a predetermined distance, placing the loopback rope wire structures in parallel, and winding the parallel structure around a mandrel to create multiple loopback electrodes. The proximal electrode 5201 and the proximal electrode 5201 positioned as uncoated regions at each end of the rope wire structure, along with the uncoated offset region 5202 and the uncoated offset region 5202 near the middle of the rope wire structure, form the stimulating proximal electrode 1 and collector electrode contacts when the loopback rope wire structure is wound on a mandrel.

The electrode systems include both helical coils and non-helical coils and structures that carry an electrical signal from a proximal end to a distal end. The electrode systems are self-anchoring, and when inserted into the body the proximal end does not protrude, making the device less obtrusive to the patient. The time of deployment of the electrode is between 5 and 30 minutes, wherein a preferred deployment time is 15 minutes. Referring to FIG. 51, the leading helical wire structure 5101 is made of conductive or non-conductive materials that allow for the formation of an anchoring bundle upon device placement. The central helical wire structure 5104, where connection wires from the standard lead ring contact electrodes 5102 are routed through an open helical coil structure, allows for the formation of an anchoring bundle upon device placement and replaces the need for placement of strain relief loops. The standard lead section 5103 and the standard lead section 5105 connect the standard lead ring contact electrodes 5102 and the standard lead ring contacts 5106 to the helical wire structure elements.

Referring to FIG. 53, FIG. 54 and FIG. 55, the connector systems interface with previously implanted electrodes and pulse generators. The helical wire structure 5301 and the helical wire structure 5301 are partially extracted through the incision 5302 and the incision 5302 in the skin 5303 to create the elongated portion 5304 and the elongated electrode 5304 at a collector site. The cylindrical conductive contact 5305 and the cylindrical conductive contact 5305 are secured to the electrode using either a crimping force or distance, or alternatively using a predetermined swaging diameter reduction to mechanically and electrically secure the cylinder to the electrode. The crimped connection 5306 and the crimped contact 5306 are formed by applying a compressive crimping force, while the swaged connection 5307 and the swaged contact 5307 are formed by applying a predetermined swaging diameter reduction.

The permanent electrode electrical and mechanical connection is made using a ferrule or segment of tubing less than 5 mm in length with an inner diameter that is greater than the diameter of the exposed electrode contact. The ferrule or tubing is manufactured from materials that do not cause galvanic corrosion with the contact material of the electrode. An insulative sleeve is either slid over the electro-mechanical connection point, or a heat shrink is applied, or an insulative adhesive is applied to limit current leakage from the connection point. Referring to FIG. 54 and FIG. 55, the electrode 5401 extends toward the implantable pulse generator 5403, with the crimping 5402 forces being applied to secure the connection. The solder pad 5404 provides a surface for establishing electrical continuity. The electrode 5501 is threaded through the primary tube 5502, and the secondary tube 5503 and the secondary tube 5504 facilitate connection to the implantable pulse generator 5403. The connection on the implantable pulse generator 5403 is direct through the use of a secondary ferrule or tube that has an inner diameter greater than that of the primary ferrule or tube, which is directly welded onto the implantable pulse generator 5403.

Referring to FIG. 60, the first cylindrical contact 6001, the second cylindrical contact 6002, and the third cylindrical contact 6003 are arranged in a linear configuration along a common axis. Individual conductors of the multi-contact helical wire structure electrode are connected to each individual cylindrical contact through welding, brazing, or using a conductive adhesive. Insulative material separating the first cylindrical contact 6001, the second cylindrical contact 6002, and the third cylindrical contact 6003 partially extends into the remainder of the helical wire structure. The multi-contact connector assembly is used for electrical stimulation or other applications such as radiofrequency ablation. Referring to FIG. 56, the ring contact 5601 and the electrode 5602 are positioned at opposite ends of a multiple contact helical wire structure electrode, with the ring contact 5601 configured to be compatible with implantable pulse generator headers. Referring to FIG. 57, multiple of the multiple contact electrodes with standard ring connectors are connected to a multiple header implantable pulse generator, extending the number of contacts in the body on nerve or other tissue targets. The implantable generator 5701 connects to the dual contact electrode 5702 to deliver electrical stimulation to target tissues.

Referring to FIG. 50 and FIG. 49, the split dual contact injectable electrode includes the helical wire structure contact 5001 and the helical wire structure contact 5002 positioned along the length of the electrode. The split of contact 1 wire rope structure 5003 and the split of contact 2 wire rope structure 5004 extend from the electrode body, with the uncoated region of contact 1 wire rope structure 5005 and the uncoated region of contact 2 wire rope structure 5006 providing exposed conductive surfaces for electrical connection. The transition point 4903 is located where the helical structure transitions to the split wire rope configuration, and the transition from helical to split structure includes additional stabilization, adhesive, or heat shrink for the transition point 4903. The dual contact injectable electrode with split end 4901 is positioned within the external cannula 4902 during delivery. The internal delivery cannula 4905 has an inner diameter that is smaller than the diameter of the helical wire structure electrode but greater than two parallel rope wire structures. The contact 1 wire rope structure 4904 and the contact 2 wire rope structure 4906 extend beyond the internal delivery cannula 4905, enabling connection to external components or pulse generators either during placement or post-placement.

Referring to FIG. 58 and FIG. 59, the interfacing solutions provide percutaneous and transcutaneous coupling between injectable electrodes and external stimulation equipment. The percutaneous injectable electrode system 5800 includes the monopolar electrode 5801 and the monopolar electrode 5802 attached to the system 5804, with the bipolar electrode 5803 shown as an alternative single electrode configuration. The electrodes are connected by way of conductive hook-and-loop connections that sufficiently secure the electrodes and provide a highly conductive electrical connection between the electrodes and the external pulse generator output via an adhesive patch adapter of the system 5804. The percutaneous injectable electrode system 5800 includes the capability to independently stimulate the electrodes versus a skin adhesive electrode to achieve a multi-polar setup, or stimulating between different contacts with 3, 4, 5, 6, 7, 8, 9, 10, 11, and 12 contacts either all separate, all multi-contact, or a mixture of both. The injectable electrode 5901 interfaces with the dual contact skin adhesive conductive hydrogel patch 5902, onto which the external pulse generator 5903 connects. The remote control 5904 provides user control functionality for the external pulse generator 5903.

Manufacturing methods for multi-contact injectable electrodes enable production of electrodes that interface with pulse generators through the connector assemblies. In an overwound bipolar injectable electrode, two mandrels are used: one for a leading electrode contact and another for a remainder of the helical wire structure beyond a second contact tissue interface. In the region between the two mandrels, the coated wire structure of the leading electrode spans the distance of the second electrode contact and serves as the mandrel in that region. To achieve contacts beyond two in a bipolar configuration, the distance of the gap between the two mandrel setup is extended to accommodate additional leads. Following each additional contact, the lead of the previous contact becomes the core of the helical wire structure while all other leads before the previous contact return to being the externally wound leads to form the helical wire structures.

Referring to FIG. 46, FIG. 47, and FIG. 48, the hook blade 4800 facilitates safe electrode removal when clinically indicated. The hook blade 4800 allows for standard cut-down approaches and facilitates device removal by introduction of the hook 4802 on the non-cutting edge of the blade. Using imaging, the alignment of the hook blade 4800 and confirmation of electrode engagement within the hook 4802 is procedurally confirmed. The cutting edge curvature 4801 extends along the outer edge of the hook blade 4800 for performing incisions through skin and tissue to access a target electrode. The hook 4802 is large enough in diameter to allow for ease of hooking onto the electrode, and is at least 1.25 times the diameter of the target electrode and up to 10 times the diameter. The inner edge of the hook 4802 has a sharpness level that cuts tissue but does not cut the electrode. The outer edge is sharper than the inner edge in order to facilitate ease of tissue cutting down to the target. The outer edge does not easily cut through the electrode in a slicing motion, allowing the electrode to withstand 1 to 20 slices from the blade prior to introducing a potential fracture point.

The blade width 4803 is the maximum transverse dimension spanning from the spine to the farthest point of the cutting edge curvature 4801, for example 6.2 mm. An entire blade length is measured from base to the sharp tip, for example 37.1 mm for a #15 blade. A blade thickness is the cross-sectional thickness of the material of the hook blade 4800, for example 0.87 mm as per sheet metal thickness capabilities. A cutting edge cross-sectional length extends from the tip along the cutting edge curvature 4801, for example 12 mm. A hook angle is the angular orientation of the hook 4802 relative to the spine of the hook blade 4800, for example 35 degrees. A hook diameter is the diameter of a circular cut forming the hook 4802, for example 1.25 mm. A hook center to edge distance is the linear distance from the tip of the hook blade 4800 to the center of the hook 4802, for example 3.0 mm. A center of hook to spine distance is the perpendicular distance from the center of the hook 4802 to the spine of the hook blade 4800, for example 0.75 mm. A steeper hook angle less than 35 degrees creates a more aggressive hook 4802 configuration but increases the risk of damaging surrounding tissue, while a shallower hook angle greater than 35 degrees results in smoother interaction with surrounding tissue but makes it probable that the electrode could slip from the hook 4802.

The devices of the current disclosure can be described as a helical wire structure electrode of the current disclosure with a loopback configuration, comprising:
a. a single wire rope structure having two ends and a middle region;
b. two proximal electrodes formed by uncoated regions at each end of the wire rope structure;
c. one distal electrode formed by an uncoated offset region located near the middle of the wire rope structure;
d, wherein the wire rope structure is folded back upon itself at the uncoated offset region to produce two parallel ropes with the two proximal electrodes offset from each other so as to not directly overlap; and
e. wherein the folded wire rope structure is wound on a core to produce a helical wire loopback structure electrode.

The helical wire structure electrode of the current disclosure, wherein the two proximal electrodes are configured for placement on an electrical stimulation target and the distal electrode is configured to serve as a collector.

The helical wire structure electrode of the current disclosure, wherein the loopback configuration is reversible such that the distal electrode formed by the uncoated offset region is placed on a stimulation target proximally, producing a helical wire loopback structure electrode with one proximal electrode and two distal electrodes.

The helical wire structure electrode of the current disclosure, wherein the helical wire structure electrode is self-anchoring and configured to form an anchoring bundle upon placement in a body.

The helical wire structure electrode of the current disclosure, wherein the helical wire structure electrode includes both helical coils and non-helical structures that carry an electrical signal from a proximal end to a distal end.

The helical wire structure electrode of the current disclosure, wherein two or more helical wire loopback structures are combined by offsetting the loopback rope wire structures at a predetermined distance, placing the loopback rope wire structures in parallel, and winding the parallel structure around a mandrel to create multiple loopback electrodes.

The helical wire structure electrode of the current disclosure, wherein when the helical wire structure electrode is inserted into a body, a proximal end does not protrude from the body.

A method for adding a connector to a previously implanted helical wire structure electrode of the current disclosure without complete removal from a body, comprising:
a. partially extracting a distal component of the implanted helical wire structure electrode through an incision in skin to extend the structure back to a non-helical rope wire structure configuration;
b. cutting the partially elongated electrode end in the rope wire structure configuration to length;
c. applying a cylindrical conductive contact by threading the wire rope structure into the cylindrical conductive contact; and
d. securing the cylindrical conductive contact to the electrode to mechanically and electrically connect the cylindrical conductive contact to the electrode.

The method of the current disclosure, wherein securing the cylindrical conductive contact comprises applying a crimping force to the cylindrical conductive contact to reduce an inner diameter of the cylindrical conductive contact and create mechanical engagement with the wire rope structure.

The method of the current disclosure, wherein securing the cylindrical conductive contact comprises applying a predetermined swaging diameter reduction to the cylindrical conductive contact.

The method of the current disclosure, wherein the cylindrical conductive contact comprises a ferrule or segment of tubing less than 5 mm in length with an inner diameter that is greater than a diameter of an exposed electrode contact.

The method of the current disclosure, wherein the ferrule or segment of tubing is manufactured from materials that do not cause galvanic corrosion with a contact material of the electrode.

The method of the current disclosure, further comprising applying an insulative sleeve over an electro-mechanical connection point, or applying a heat shrink, or applying an insulative adhesive to limit current leakage from the connection point.

The method of the current disclosure, wherein a proximal end of the electrode remains unmoved at an original implantation site while the distal component receives the cylindrical conductive contact, and wherein the helical wire structure electrode is converted to an electrode configuration that interfaces with a socket connector of an implantable pulse generator.

A hook blade device of the current disclosure for percutaneous removal of an injectable electrode, comprising:
a. a blade having a spine, a tip, and a cutting edge curvature on an outer edge configured for performing skin and tissue cutting down to a target electrode; and
b. a hook positioned on a non-cutting edge of the blade, the hook configured to engage with and retrieve the target electrode.

The hook blade device of the current disclosure, wherein the hook has a diameter that is at least 1.25 times a diameter of the target electrode and up to 10 times the diameter of the target electrode.

The hook blade device of the current disclosure, wherein an inner edge of the hook has a sharpness level that cuts tissue but does not cut the target electrode, and wherein an outer edge of the blade is sharper than the inner edge of the hook.

The hook blade device of the current disclosure, wherein the outer edge does not cut through the target electrode in a slicing motion, allowing the target electrode to withstand 1 to 20 slices from the blade prior to introducing a potential fracture point.

The hook blade device of the current disclosure, wherein a hook angle is an angular orientation of the hook relative to the spine of the blade, and wherein the hook angle is approximately 35 degrees.

The hook blade device of the current disclosure, wherein a steeper hook angle less than 35 degrees creates a more aggressive hook configuration but increases a risk of damaging surrounding tissue, and wherein a shallower hook angle greater than 35 degrees results in smoother interaction with surrounding tissue but increases a probability that the target electrode could slip from the hook.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A helical wire structure electrode with a loopback configuration, comprising:
a single wire rope structure having two ends and a middle region;
two proximal electrodes formed by uncoated regions at each end of the wire rope structure;
one distal electrode formed by an uncoated offset region located near the middle of the wire rope structure;
wherein the wire rope structure is folded back upon itself at the uncoated offset region to produce two parallel ropes with the two proximal electrodes offset from each other so as to not directly overlap; and
wherein the folded wire rope structure is wound on a core to produce a helical wire loopback structure electrode.

2. The helical wire structure electrode of claim 1, wherein the two proximal electrodes are configured for placement on an electrical stimulation target and the distal electrode is configured to serve as a collector.

3. The helical wire structure electrode of claim 1, wherein the loopback configuration is reversible such that the distal electrode formed by the uncoated offset region is placed on a stimulation target proximally, producing a helical wire loopback structure electrode with one proximal electrode and two distal electrodes.

4. The helical wire structure electrode of claim 1, wherein the helical wire structure electrode is self-anchoring and configured to form an anchoring bundle upon placement in a body.

5. The helical wire structure electrode of claim 1, wherein the helical wire structure electrode includes both helical coils and non-helical structures that carry an electrical signal from a proximal end to a distal end.

6. The helical wire structure electrode of claim 1, wherein two or more helical wire loopback structures are combined by offsetting the loopback rope wire structures at a predetermined distance, placing the loopback rope wire structures in parallel, and winding the parallel structure around a mandrel to create multiple loopback electrodes.

7. The helical wire structure electrode of claim 1, wherein when the helical wire structure electrode is inserted into a body, a proximal end does not protrude from the body.

8. A method for adding a connector to a previously implanted helical wire structure electrode without complete removal from a body, comprising:
partially extracting a distal component of the implanted helical wire structure electrode through an incision in skin to extend the structure back to a non-helical rope wire structure configuration;
cutting the partially elongated electrode end in the rope wire structure configuration to length;
applying a cylindrical conductive contact by threading the wire rope structure into the cylindrical conductive contact; and
securing the cylindrical conductive contact to the electrode to mechanically and electrically connect the cylindrical conductive contact to the electrode.

9. The method of claim 8, wherein securing the cylindrical conductive contact comprises applying a crimping force to the cylindrical conductive contact to reduce an inner diameter of the cylindrical conductive contact and create mechanical engagement with the wire rope structure.

10. The method of claim 8, wherein securing the cylindrical conductive contact comprises applying a predetermined swaging diameter reduction to the cylindrical conductive contact.

11. The method of claim 8, wherein the cylindrical conductive contact comprises a ferrule or segment of tubing less than 5 mm in length with an inner diameter that is greater than a diameter of an exposed electrode contact.

12. The method of claim 11, wherein the ferrule or segment of tubing is manufactured from materials that do not cause galvanic corrosion with a contact material of the electrode.

13. The method of claim 8, further comprising applying an insulative sleeve over an electro-mechanical connection point, or applying a heat shrink, or applying an insulative adhesive to limit current leakage from the connection point.

14. The method of claim 8, wherein a proximal end of the electrode remains unmoved at an original implantation site while the distal component receives the cylindrical conductive contact, and wherein the helical wire structure electrode is converted to an electrode configuration that interfaces with a socket connector of an implantable pulse generator.

15. A hook blade device for percutaneous removal of an injectable electrode, comprising:
a blade having a spine, a tip, and a cutting edge curvature on an outer edge configured for performing skin and tissue cutting down to a target electrode; and
a hook positioned on a non-cutting edge of the blade, the hook configured to engage with and retrieve the target electrode.

16. The hook blade device of claim 15, wherein the hook has a diameter that is at least 1.25 times a diameter of the target electrode and up to 10 times the diameter of the target electrode.

17. The hook blade device of claim 15, wherein an inner edge of the hook has a sharpness level that cuts tissue but does not cut the target electrode, and wherein an outer edge of the blade is sharper than the inner edge of the hook.

18. The hook blade device of claim 15, wherein the outer edge does not cut through the target electrode in a slicing motion, allowing the target electrode to withstand 1 to 20 slices from the blade prior to introducing a potential fracture point.

19. The hook blade device of claim 15, wherein a hook angle is an angular orientation of the hook relative to the spine of the blade, and wherein the hook angle is approximately 35 degrees.

20. The hook blade device of claim 19, wherein a steeper hook angle less than 35 degrees creates a more aggressive hook configuration but increases a risk of damaging surrounding tissue, and wherein a shallower hook angle greater than 35 degrees results in smoother interaction with surrounding tissue but increases a probability that the target electrode could slip from the hook.

21. The hook blade device of claim 15, where an electrical connection between the blade and an electrical return is analyzed by an impedance spectrometer to determine whether the hook blade has made contact with a conductive implanted electrode and providing visual, audible, or tactile feedback to the clinical user that contact has been made.

22. The hook blade device of claim 16, where a piezoelectric sensor is connected to the hook blade in order to sense physical contact made between the blade and an implanted device through changes in vibration frequency and amplitude profiles.
